SEQUENCE LISTING (42 sequences)

SEQ ID NO: 1 - DNA, 2004 bp, Artificial Sequence
  Human Zbtb20 cDNA
atgaccgagcgcattcacagcatcaaccttcacaacttcagcaattccgtgctcgagaccctcaacgagcagcgcaaccgtggccacttctgtgacgtaacggtgcgcatccacgggagcatgctgcgcgcacaccgctgcgtgctggcagccggcagccccttcttccaggacaaactgctgcttggctacagcgacatcgagatcccgtcggtggtgtcagtgcagtcagtgcaaaagctcattgacttcatgtacagcggcgtgctacgggtctcgcagtcggaagctctgcagatcctcacggccgccagcatcctgcagatcaaaacagtcatcgacgagtgcacgcgcatcgtgtcacagaacgtgggcgatgtgttcccggggatccaggactcgggccaggacacgccgcggggcactcccgagtcaggcacgtcaggccagagcagcgacacggagtcgggctacctgcagagccacccacagcacagcgtggacaggatctactcggcactctacgcgtgctccatgcagaatggcagcggcgagcgctctttttacagcggcgcagtggtcagccaccacgagactgcgctcggcctgccccgcgaccaccacatggaagaccccagctggatcacacgcatccatgagcgctcgcagcagatggagcgctacctgtccaccacccccgagaccacgcactgccgcaagcagccccggcctgtgcgcatccagaccctagtgggcaacatccacatcaagcaggagatggaggacgattacgactactacgggcagcaaagggtgcagatcctggaacgcaacgaatccgaggagtgcacggaagacacagaccaggccgagggcaccgagagtgagcccaaaggtgaaagcttcgactcgggcgtcagctcctccataggcaccgagcctgactcggtggagcagcagtttgggcctggggcggcgcgggacagccaggctgaacccacccaacccgagcaggctgcagaagcccccgctgagggtggtccgcagacaaaccagctagaaacaggtgcttcctctccggagagaagcaatgaagtggagatggacagcactgttatcactgtcagcaacagctccgacaagagcgtcctacaacagccttcggtcaacacgtccatcgggcagccattgccaagtacccagctctacttacgccagacagaaaccctcaccagcaacctgaggatgcctctgaccttgaccagcaacacgcaggtcattggcacagctggcaacacctacctgccagccctcttcactacccagcccgcgggcagtggccccaagcctttcctcttcagcctgccacagcccctggcaggccagcagacccagtttgtgacagtgttccagcccggtctgtcgacctttactgcacagctgccagcgccacagcccctggcctcatccgcaggccacagcacagccagtgggcaaggcgaaaaaaagccttatgagtgcactctctgcaacaagactttcaccgccaaacagaactacgtcaagcacatgttcgtacacacaggtgagaagccccaccaatgcagcatctgttggcgctccttctccttaaaggattaccttatcaagcacatggtgacacacacaggagtgagggcataccagtgtagtatctgcaacaagcgcttcacccagaagagctccctcaacgtgcacatgcgcctccaccggggagagaagtcctacgagtgctacatctgcaaaaagaagttctctcacaagaccctcctggagcgacacgtggccctgcacagtgccagcaatgggaccccccctgcaggcacacccccaggtgcccgcgctggccccccaggcgtggtggcctgcacggaggggaccacttacgtctgctccgtctgcccagcaaagtttgaccaaatcgagcagttcaacgaccacatgaggatgcatgtgtctgacgga

SEQ ID NO: 2 - protein, 668 aa, Artificial Sequence
  Human Zbtb20
MTERIHSINLHNFSNSVLETLNEQRNRGHFCDVTVRIHGSMLRAHRCVLAAGSPFFQDKLLLGYSDIEIPSVVSVQSVQKLIDFMYSGVLRVSQSEALQILTAASILQIKTVIDECTRIVSQNVGDVFPGIQDSGQDTPRGTPESGTSGQSSDTESGYLQSHPQHSVDRIYSALYACSMQNGSGERSFYSGAVVSHHETALGLPRDHHMEDPSWITRIHERSQQMERYLSTTPETTHCRKQPRPVRIQTLVGNIHIKQEMEDDYDYYGQQRVQILERNESEECTEDTDQAEGTESEPKGESFDSGVSSSIGTEPDSVEQQFGPGAARDSQAEPTQPEQAAEAPAEGGPQTNQLETGASSPERSNEVEMDSTVITVSNSSDKSVLQQPSVNTSIGQPLPSTQLYLRQTETLTSNLRMPLTLTSNTQVIGTAGNTYLPALFTTQPAGSGPKPFLFSLPQPLAGQQTQFVTVFQPGLSTFTAQLPAPQPLASSAGHSTASGQGEKKPYECTLCNKTFTAKQNYVKHMFVHTGEKPHQCSICWRSFSLKDYLIKHMVTHTGVRAYQCSICNKRFTQKSSLNVHMRLHRGEKSYECYICKKKFSHKTLLERHVALHSASNGTPPAGTPPGARAGPPGVVACTEGTTYVCSVCPAKFDQIEQFNDHMRMHVSDG

SEQ ID NO: 3 - DNA, 2223 bp, Artificial Sequence
  Mouse Zbtb20 cDNA
atgctagaacggaagaaacccaagacagctgaaaaccagaaggcatctgaggagaatgagattactcagccgggcggatccagcgccaagccggcccttccctgcctgaactttgaagctgttttgtctccagccccagccctcatccactcgacacattcactgacaaactctcacgctcacaccgggtcatctgattgtgacatcagttgcaaggggatgaccgagcgcattcacagcatcaaccttcacaacttcagcaattccgtgctcgagaccctcaacgagcagcgcaaccgtggccacttctgtgacgtgacggttcgcatccacgggagcatgctgcgcgcacatcgctgcgtgctggcagccggcagccccttcttccaagacaagctgctgctgggctacagcgacatcgaaatcccgtcggtggtgtccgtacaatcggtgcaaaagctcattgacttcatgtacagcggtgtgctgagagtctcacagtcggaagctctgcagatcctcacagccgccagcatcctgcagatcaaaacagtcatagatgagtgcactcgcatcgtgtcacagaacgtgggcgatgtgttcccaggcatccaggattctggccaggacacaccaagaggcacaccagagtcaggcacatctggccagagcagtgacacggaatcaggctacctgcagagccacccacagcatagtgtggaccgaatctactccgcactctacgcctgctccatgcagaatggcagcggcgagcgctccttctacagtggtgcagtggtcagccaccacgaaacagctctcggcctgccccgtgaccaccacatggaagaccctagctggatcacacgcattcatgagcgctcccagcaaatggagcgctacctgtccaccacccctgagaccacgcactgccggaagcagccccggcctgtgcgtatccagaccctggtgggtaacatccacatcaagcaggaaatggaagatgactatgactactatgggcagcaaagggtgcagatcctagaacgcaatgaatccgaggagtgcacagaagacactgaccaagcagagggcactgagagcgagcccaaaggtgaaagctttgattctggggtcagctcctccatcggcaccgaacctgactcagtggagcaacagtttggggcagcagccccaagggacggtcaggcagaacccgcccaacctgagcaggcagcagaagccccagctgagagcagtgcccagccaaaccagctagaaccaggtgcctcctctcctgagagaagcaacgagtcagagatggacaacacagtcatcactgtcagtaacagctccgataagggcgtcctacagcagccttcagtcaacacatccatcgggcagccattgccaagtacccagctctatttacgccagacagaaaccctcaccagcaacctgaggatgcctctgaccttgaccagcaacacacaggtcattggcaccgctggcaacacctatctgccagccctcttcactacccaacccgcgggcagtggccccaagccttttctcttcagcctgccgcagcccctgacaggccagcagacccagtttgtgacagtgtcccagcccggtctgtccacctttactgcacagctgccagcgccacagcccctggcctcatctgcaggccacagcacagccagtgggcaaggcgacaaaaagccttatgagtgcactctctgcaacaagactttcacagccaaacagaactacgtcaagcacatgttcgtacatacaggtgagaagccccaccagtgcagcatctgctggcgctccttctccttgaaggattaccttatcaagcacatggtgacgcacaccggcgtgagagcgtaccagtgtagcatctgcaacaagcgcttcacccagaagagttccctcaacgtgcacatgcgcctgcaccgcggggagaagtcctatgagtgctacatctgcaaaaagaagttctcccacaagaccctgctggagcgacacgtggccctgcacagtgccagcaacgggacccctccggcaggcacgcccccaggtgcccgcgcgggtccgccaggcgtggtggcctgcacagaggggaccacttacgtctgctccgtctgcccagcaaagtttgaccaaatcgagcagttcaacgaccacatgaggatgcatgtgtctgacgga

SEQ ID NO: 4 - protein, 741 aa, Artificial Sequence
  Mouse Zbtb20
MLERKKPKTAENQKASEENEITQPGGSSAKPALPCLNFEAVLSPAPALIHSTHSLTNSHAHTGSSDCDISCKGMTERIHSINLHNFSNSVLETLNEQRNRGHFCDVTVRIHGSMLRAHRCVLAAGSPFFQDKLLLGYSDIEIPSVVSVQSVQKLIDFMYSGVLRVSQSEALQILTAASILQIKTVIDECTRIVSQNVGDVFPGIQDSGQDTPRGTPESGTSGQSSDTESGYLQSHPQHSVDRIYSALYACSMQNGSGERSFYSGAVVSHHETALGLPRDHHMEDPSWITRIHERSQQMERYLSTTPETTHCRKQPRPVRIQTLVGNIHIKQEMEDDYDYYGQQRVQILERNESEECTEDTDQAEGTESEPKGESFDSGVSSSIGTEPDSVEQQFGAAAPRDGQAEPAQPEQAAEAPAESSAQPNQLEPGASSPERSNESEMDNTVITVSNSSDKGVLQQPSVNTSIGQPLPSTQLYLRQTETLTSNLRMPLTLTSNTQVIGTAGNTYLPALFTTQPAGSGPKPFLFSLPQPLTGQQTQFVTVSQPGLSTFTAQLPAPQPLASSAGHSTASGQGDKKPYECTLCNKTFTAKQNYVKHMFVHTGEKPHQCSICWRSFSLKDYLIKHMVTHTGVRAYQCSICNKRFTQKSSLNVHMRLHRGEKSYECYICKKKFSHKTLLERHVALHSASNGTPPAGTPPGARAGPPGVVACTEGTTYVCSVCPAKFDQIEQFNDHMRMHVSDG

SEQ ID NO: 5 - DNA, 57 bp, Artificial Sequence
  DNA encoding shRNA targeting human Zbtb20 transcript
ccggcgcagacaaaccagctagaaactcgagtttctagctggtttgtctgcgttttt

SEQ ID NO: 6 - RNA, 57 nt, Artificial Sequence
  shRNA targeting human Zbtb20 transcript
ccggcgcagacaaaccagcuagaaacucgaguuucuagcugguuugucugcguuuuu

SEQ ID NO: 7 - DNA, 57 bp, Artificial Sequence
  DNA encoding shRNA targeting human Zbtb20 transcript
ccggcccagcaaagtttgaccaaatctcgagatttggtcaaactttgctgggttttt

SEQ ID NO: 8 - RNA, 57 nt, Artificial Sequence
  shRNA targeting human Zbtb20 transcript
ccggcccagcaaaguuugaccaaaucucgagauuuggucaaacuuugcuggguuuuu

SEQ ID NO: 9 - DNA, 58 bp, Artificial Sequence
  DNA encoding shRNA targeting human Zbtb20 transcript
ccggcgggtcatctgattgtgacatctcgagatgtcacaatcagatgacccgtttttg

SEQ ID NO: 10 - RNA, 58 nt, Artificial Sequence
  shRNA targeting human Zbtb20 transcript
ccggcgggucaucugauugugacaucucgagaugucacaaucagaugacccguuuuug

SEQ ID NO: 11 - DNA, 58 bp, Artificial Sequence
  DNA encoding shRNA targeting mouse Zbtb20 transcript
ccgggggctacagcgacatcgaaatctcgagatttcgatgtcgctgtagccctttttg

SEQ ID NO: 12 - RNA, 58 nt, Artificial Sequence
  shRNA targeting mouse Zbtb20 transcript
ccgggggcuacagcgacaucgaaaucucgagauuucgaugucgcuguagcccuuuuug

SEQ ID NO: 13 - DNA, 58 bp, Artificial Sequence
  DNA encoding shRNA targeting mouse Zbtb20 transcript
ccgggcctgctggtacattacatttctcgagaaatgtaatgtaccagcaggctttttg

SEQ ID NO: 14 - RNA, 58 nt, Artificial Sequence
  shRNA targeting mouse Zbtb20 transcript
ccgggccugcugguacauuacauuucucgagaaauguaauguaccagcaggcuuuuug

SEQ ID NO: 15 - DNA, 58 bp, Artificial Sequence
  DNA encoding shRNA targeting mouse Zbtb20 transcript
ccggagctatggcactagaatttaactcgagttaaattctagtgccatagcttttttg

SEQ ID NO: 16 - RNA, 58 nt, Artificial Sequence
  shRNA targeting mouse Zbtb20 transcript
ccggagcuauggcacuagaauuuaacucgaguuaaauucuagugccauagcuuuuuug

SEQ ID NO: 17 - DNA, 20 bp, Artificial Sequence
  DNA encoding sgRNA targeting human Zbtb20 gene
gttgatgctgtgaatgcgct

SEQ ID NO: 18 - RNA, 20 nt, Artificial Sequence
  sgRNA targeting human Zbtb20 gene
guugaugcugugaaugcgcu

SEQ ID NO: 19 - DNA, 20 bp, Artificial Sequence
  DNA encoding sgRNA targeting human Zbtb20 gene
cggaattgctgaagttgtga

SEQ ID NO: 20 - RNA, 20 nt, Artificial Sequence
  sgRNA targeting human Zbtb20 gene
cggaauugcugaaguuguga

SEQ ID NO: 21 - DNA, 20 bp, Artificial Sequence
  DNA encoding sgRNA targeting human Zbtb20 gene
ctcgttgagggtctcgagca

SEQ ID NO: 22 - RNA, 20 nt, Artificial Sequence
  sgRNA targeting human Zbtb20 gene
cucguugagggucucgagca

SEQ ID NO: 23 - DNA, 20 bp, Artificial Sequence
  DNA encoding sgRNA targeting human Zbtb20 gene
acggttgcgctgctcgttga

SEQ ID NO: 24 - RNA, 20 nt, Artificial Sequence
  sgRNA targeting human Zbtb20 gene
acgguugcgcugcucguuga

SEQ ID NO: 25 - DNA, 20 bp, Artificial Sequence
  DNA encoding sgRNA targeting mouse Zbtb20 gene
caagacagctgaaaaccaga

SEQ ID NO: 26 - RNA, 20 nt, Artificial Sequence
  sgRNA targeting mouse Zbtb20 gene
caagacagcugaaaaccaga

SEQ ID NO: 27 - DNA, 20 bp, Artificial Sequence
  DNA encoding sgRNA targeting mouse Zbtb20 gene
tgaaaaccagaaggcatctg

SEQ ID NO: 28 - RNA, 20 nt, Artificial Sequence
  sgRNA targeting mouse Zbtb20 gene
ugaaaaccagaaggcaucug

SEQ ID NO: 29 - DNA, 20 bp, Artificial Sequence
  DNA encoding sgRNA targeting mouse Zbtb20 gene
ggagaatgagattactcagc

SEQ ID NO: 30 - RNA, 20 nt, Artificial Sequence
  sgRNA targeting mouse Zbtb20 gene
ggagaaugagauuacucagc

SEQ ID NO: 31 - DNA, 20 bp, Artificial Sequence
  DNA encoding sgRNA targeting mouse Zbtb20 gene
gagaatgagattactcagcc

SEQ ID NO: 32 - RNA, 20 nt, Artificial Sequence
  sgRNA targeting mouse Zbtb20 gene
gagaaugagauuacucagcc

SEQ ID NO: 33 - DNA, 20 bp, Artificial Sequence
  DNA encoding sgRNA targeting human Zbtb20 promoter
acttactctttctgctcggg

SEQ ID NO: 34 - RNA, 20 nt, Artificial Sequence
  sgRNA targeting human Zbtb20 promoter
acuuacucuuucugcucggg

SEQ ID NO: 35 - DNA, 20 bp, Artificial Sequence
  DNA encoding sgRNA targeting human Zbtb20 promoter
ccagcatgagctggaaatgt

SEQ ID NO: 36 - RNA, 20 nt, Artificial Sequence
  sgRNA targeting human Zbtb20 promoter
ccagcaugagcuggaaaugu

SEQ ID NO: 37 - DNA, 20 bp, Artificial Sequence
  DNA encoding sgRNA targeting human Zbtb20 promoter
cggtacagtccagcatgagc

SEQ ID NO: 38 - RNA, 20 nt, Artificial Sequence
  sgRNA targeting human Zbtb20 promoter
cgguacaguccagcaugagc

SEQ ID NO: 39 - DNA, 645 bp, Artificial Sequence
  Human dominant negative Zbtb20 cDNA nucleotide sequence
atgctgccacagcccctggcaggccagcagacccagtttgtgacagtgttccagcccggtctgtcgacctttactgcacagctgccagcgccacagcccctggcctcatccgcaggccacagcacagccagtgggcaaggcgaaaaaaagccttatgagtgcactctctgcaacaagactttcaccgccaaacagaactacgtcaagcacatgttcgtacacacaggtgagaagccccaccaatgcagcatctgttggcgctccttctccttaaaggattaccttatcaagcacatggtgacacacacaggagtgagggcataccagtgtagtatctgcaacaagcgcttcacccagaagagctccctcaacgtgcacatgcgcctccaccggggagagaagtcctacgagtgctacatctgcaaaaagaagttctctcacaagaccctcctggagcgacacgtggccctgcacagtgccagcaatgggaccccccctgcaggcacacccccaggtgcccgcgctggccccccaggcgtggtggcctgcacggaggggaccacttacgtctgctccgtctgcccagcaaagtttgaccaaatcgagcagttcaacgaccacatgaggatgcatgtgtctgacgga

SEQ ID NO: 40 - protein, 215 aa, Artificial Sequence
  Human dominant negative Zbtb20 amino acid sequence
MLPQPLAGQQTQFVTVFQPGLSTFTAQLPAPQPLASSAGHSTASGQGEKKPYECTLCNKTFTAKQNYVKHMFVHTGEKPHQCSICWRSFSLKDYLIKHMVTHTGVRAYQCSICNKRFTQKSSLNVHMRLHRGEKSYECYICKKKFSHKTLLERHVALHSASNGTPPAGTPPGARAGPPGVVACTEGTTYVCSVCPAKFDQIEQFNDHMRMHVSDG

SEQ ID NO: 41 - DNA, 645 bp, Artificial Sequence
  Mouse dominant negative Zbtb20 cDNA nucleotide sequence
atgctgccgcagcccctgacaggccagcagacccagtttgtgacagtgtcccagcccggtctgtccacctttactgcacagctgccagcgccacagcccctggcctcatctgcaggccacagcacagccagtgggcaaggcgacaaaaagccttatgagtgcactctctgcaacaagactttcacagccaaacagaactacgtcaagcacatgttcgtacatacaggtgagaagccccaccagtgcagcatctgctggcgctccttctccttgaaggattaccttatcaagcacatggtgacgcacaccggcgtgagagcgtaccagtgtagcatctgcaacaagcgcttcacccagaagagttccctcaacgtgcacatgcgcctgcaccgcggggagaagtcctatgagtgctacatctgcaaaaagaagttctcccacaagaccctgctggagcgacacgtggccctgcacagtgccagcaacgggacccctccggcaggcacgcccccaggtgcccgcgcgggtccgccaggcgtggtggcctgcacagaggggaccacttacgtctgctccgtctgcccagcaaagtttgaccaaatcgagcagttcaacgaccacatgaggatgcatgtgtctgacgga

SEQ ID NO: 42 - protein, 215 aa, Artificial Sequence
  Mouse dominant negative Zbtb20 amino acid sequence
MLPQPLTGQQTQFVTVSQPGLSTFTAQLPAPQPLASSAGHSTASGQGDKKPYECTLCNKTFTAKQNYVKHMFVHTGEKPHQCSICWRSFSLKDYLIKHMVTHTGVRAYQCSICNKRFTQKSSLNVHMRLHRGEKSYECYICKKKFSHKTLLERHVALHSASNGTPPAGTPPGARAGPPGVVACTEGTTYVCSVCPAKFDQIEQFNDHMRMHVSDG

(19) United States
(12) Patent Application Publication
(10) Pub. No.: US 2023/0042446 A1
(43) Pub. Date: Feb. 9, 2023

(54) ADOPTIVE CELL THERAPY WITH ZBTB20 SUPPRESSION

(71) Applicant: THE TRUSTEES OF DARTMOUTH COLLEGE, Hanover, NH (US)

(72) Inventors: Edward USHERWOOD, Hanover, NH (US); Young-Kwang USHERWOOD, Hanover, NH (US)

(21) Appl. No.: 17/782,252

(22) PCT Filed: Dec. 4, 2020
(86) PCT No.: PCT/US2020/063291

§ 371 Date: Jun. 3, 2022

Related U.S. Application Data
(60) Provisional application No. 62/943,526, filed on Dec. 4, 2019.

(51) Int. Cl. A61K 35/17; C07K 14/47; C12N 15/113; C12N 15/11; A61K 45/06; A61P 35/00

(52) U.S. Cl. CPC A61K 35/17; C07K 14/4702; C12N 15/1138; C12N 15/11; A61K 45/06; A61P 35/00; C12N 2310/20; C12N 2310/14; C12N 2310/531

(57) ABSTRACT

Provided are methods, compositions, and cells for use in adoptive cell therapy for the treatment of cancer. The methods involve administering an effective amount of cells to a subject, wherein the cells are modified ex vivo to suppress endogenous Zbtb20 expression and/or activity within the modified cells. The cells may comprise a dominant negative Zbtb20 capable of suppressing endogenous Zbtb20 activity, at least one shRNA capable of suppressing endogenous Zbtb20 expression, or at least one sgRNA capable of suppressing endogenous Zbtb20 expression. The cells may further comprise an exogenous TCR and/or CAR suitable for treating cancer. The method can further involve administering one or more additional cancer therapies, such as cells which express at least one exogenous TCR and/or CAR suitable for treating cancer. The method can provide various advantages, such as a reduction and/or elimination of an amount of cancer cells in the subject.

### RELATED APPLICATIONS

This invention claims priority to U.S. Provisional Application No. 62/943,526, filed on Dec. 4, 2019, the contents of which are incorporated by reference in their entirety herein.

### STATEMENT OF FEDERALLY FUNDED RESEARCH

This invention was made with government support under Grant Nos. P30 GM103415 and RO1 AI122854 awarded by the National Institutes of Health. The U.S. government has certain rights in the invention.

### REFERENCE TO A SEQUENCE LISTING

The present application includes a Sequence Listing which has been submitted in ASCII format via EFS-Web and is hereby incorporated by reference in its entirety. Said ASCII copy, created on Nov. 22, 2019, is named 1143252o004200.txt and is 30.7 KB in size.

### FIELD OF THE ART

The present disclosure generally relates to the field of adoptive cell therapy, and more particularly, to cells, compositions, and methods for adoptive cell therapy with Zbtb20 suppression. As such, the present disclosure relates to nucleic acids and proteins suitable for suppressing Zbtb20 expression and/or activity in cells and to modified cells in which endogenous Zbtb20 expression and/or activity is suppressed. The present disclosure also generally relates to compositions containing said modified cells and methods of use thereof in adoptive cell therapy, in particular for treating cancer and for slowing and/or reversing the growth of tumor cells in a subject.

### BACKGROUND

Cancer immunotherapy is defined as the approach to combatting cancer by generating or augmenting an immune response against cancer cells. Over the past decade, two types of immunotherapy have emerged as particularly effective in cancer treatment: the use of immune checkpoint inhibitors to enhance natural antitumor activity and the administration of specific antitumor immune cells via adoptive cell therapy (ACT) (Met, et al., Seminars in Immunopathology, 41(1):49-58).

### Immune Checkpoint Inhibitors

Currently, the most commonly used type of immunotherapy is known as immune checkpoint inhibitors monoclonal antibodies directed against regulatory immune checkpoint factors that inhibit T cell activation. These factors include programmed cell death-1 (PD-1), programmed death-ligand 1 (PD-L1), and cytotoxic T lymphocyte-associated protein-4 (CTLA-4). Immune checkpoint inhibitors have been successful for improving overall and disease-free survival in multiple clinical trials, including ipilimumab and Nivolumab for melanomas (Hodi et al., N Engl J Med 363:711-723; Robert et al., N Engl J Med 372:320-330; and Larkin, et al. N Engl J Med 373:23-34), Pembrolizumab for non-small-cell lung cancer (Garon, et al., N Engl J Med 372:2018-2028) and for head and neck cancer (Bauml, et al., J Clin Oncol 35:1542-1549), and Nivolumab for urothelial carcinoma (Sharma, et al., Lancet Oncol 17:1590-1598) and for Hodgkin's lymphoma (Ansell, et al., N Engl J Med 372:311-319).

### Adoptive Cell Therapy

Another type of immunotherapy known as adoptive cell therapy (ACT) involves ex vivo manipulation and expansion of cells, typically T cells, derived from a patient and subsequent reinfusion of the T cells into the patient to generate a robust immune-mediated response. ACT-based strategies can be derived from (i) tumor-infiltrating lymphocyte (TIL) T cells isolated from the patient's tumors and which specifically recognize the patient's tumor cells, and (ii) genetically modified T cells derived from the patient's blood to enable specific recognition of the patient's tumor cells. The genetic modification generally comprises introduction of (a) an exogenous T cell receptor (TCR) or (b) a chimeric antigen receptor (CAR). Additionally, B cell-based adoptive cell therapies is also an emerging approach in cancer immunotherapy which has been shown to be generally safe and associated with little toxicity, and which can elicit antitumor T cell responses (Wennhold et al., Transfus Med Hemother 2019; 46:36-46).

Adoptive cell therapies can be effective on their own or can complement and enhance immune checkpoint inhibitor therapy for patients with poorly immunogenic cancer types and/or patients whose tumors already respond to immune checkpoint inhibitors. In addition to immunotherapy, ACT can also be used in conjunction with other cancer therapies, including chemotherapy, targeted therapy, stem cell transplant, radiation, surgery, and hormone therapy.

### ACT Using Tumor-infiltrating Lymphocytes

TILs comprise endogenous T cell receptors (TCRs) which recognizing tumor associated antigens present on a patient's tumors. A standard method for large-scale ex vivo expansion of TILs isolated from patient tumors has been developed and involves culturing the TILs with a high dose of the T cell growth factor interleukin-2 (IL-2) followed by a rapid expansion process utilizing a mixed feeder cell population (Rosenberg, et al., 1988, N Engl J Med 319:1676-1680).

TIL therapy involves nonmyeloablative lymphodepletion prior to cell infusion, commonly including cyclophosphamide and fludarabine. This preconditioning regimen increases the persistence of infused TILs and improves clinical responses after TIL therapy. After infusion of the ex-vivo expanded TILs, the patient receives IL-2 (Dudley et al., 2003, J Immunother 26:332-342 and Dudley et al., 2005, J Clin Oncol 23:2346-2357).

For the ex vivo TIL expansion step, a resected tumor specimen is divided into multiple fragments that are individually grown in IL-2 or enzymatically dispersed into a single-cell suspension. The lymphocytes from the specimen overgrow and typically eradicate tumor cells within 2-3 weeks, resulting in pure TIL cultures. If autologous tumor cells are available, individual TIL cultures can be selected based on attributes such as tumor-reactive interferon-γ (IFN-γ) secretion and cytotoxicity. Selected TIL cultures are then subjected to a rapid expansion protocol (REP) in the presence of excess irradiated feeder cells, an antibody targeting the CD3 complex of the tumor-specific endogenous TCR, and high dose IL-2. With this approach, up to 2×10\hat{ }¹¹ lymphocytes can be obtained for reinfusion into patients (Andersen et al., 2018, Ann Oncol 29(7):1575-1581). However, difficulties in generating autologous tumor cultures and variations in target tumor quality have prompted many institutions to utilize minimally cultured TILs, where typically all isolated TILs are utilized for further massive expansion and infusion (Tran et al., 2008, J Immunother 31:742-751; Donia et al., 2012, Scand J Immunol 75:157-167; and Besser et al., 2009, J Immunother 32:415-423). The main benefit of this approach is the considerably reduced culture period, which simplifies a significant portion of this complex expansion platform and is less labor-intensive and more cost-effective.

TIL-based ACT has been largely successful in certain trials, including those for metastatic melanoma and cervical cancer (Rosenberg, et al., 1988, N Engl J Med 319:1676-1680; Dudley, et al., 2005, J Clin Oncol 23:2346-2357; Itzhaki et al., 2011, J Immunother 34:212-220; Radvanyi, et al., 2012, Clin Cancer Res 18:6758-6770; Andersen, et al, 2018, Clin Cancer Res 22:3734-3745; and Hilders, et al., 2003, Int J Cancer 57:805-813). Whereas LN-144 (lifileucel) has not yet received FDA approval for melanoma patents, LN-145 has recently been approved for treating cervical cancer. This has prompted TIL-based ACT trials for other solid cancers, including ovarian, breast, colon, sarcoma, and renal (Webb, et al., Clin Cancer Res 20:434-444; Yannelli, et al. Int J Cancer 65:413-421; Turcotte et al., J Immunol 191:2217-2225; and Andersen, et al., 2018, Cancer Immunol Res 6:222-235); however, only moderate clinical responses have been observed. As such, improvements in TIL-based ACT methods are needed.

### ACT Using Genetically Modified T Cells

Genetically modified T cells represent an alternative approach for generating tumor-specific T cell therapies to enhance antitumor immune function. The approach involves ex vivo genetic engineering of T cells to express an exogenous T cell receptor (TCR) or a synthetic chimeric antigen receptor (CAR) targeting tumor specific antigens. A CAR comprises the antigen-binding portions of an antibody and the signaling components of various immunoreceptors and costimulatory molecules. CARs are designed for optimal specificity and reactivity.

For either exogenous TCR or CAR T cell therapy, T cells are obtained from peripheral blood, usually after leukapheresis, activated ex vivo, genetically engineered, and expanded prior to their reinfusion back into the patient. The patient usually receives a preconditioning regimen similar to that of TIL-based ACT prior to reinfusion.

### Exogenous TCR Therapy

TCRs naturally recognize peptide antigens presented on the surface of host cells via the major histocompatibility complex (MHC)/human leukocyte antigen (HLA) system. Each TCR comprises two disulfide-linked glycoprotein chains (usually a and a chains) having constant and variable regions which recognize antigens. Accessory CD3 transmembrane and intracellular signaling domains facilitate signaling. For exogenous TCR therapy, peripheral blood T cells are genetically engineered ex vivo with a recombinant TCR having tumor antigen-specific α and β chains. This is often achieved via expression of the exogenous TCR from a retro- or lentiviral vector.

One limitation of this approach is that because TCRs bind to peptide/MHC complexes at the cell surface of tumor cells, the exogenous tumor-specific TCRs can only be used in a patient population that has this specific MHC or HLA allele. Further, tumor antigen-specific T cells targeting self-antigens isolated from cancer patients are of low affinity, due to the impact of central tolerance on the T cell repertoire specific for these antigens. Attempts to overcome this issue have included (i) engineering of high affinity TCRs by affinity maturation of the TCR, (ii) generation of murine TCRs by immunizing transgenic mice that express an HLA allele plus human tumor antigen, and (iii) isolation of TCRs in an allogeneic setting via in vitro induction of T cells specific for a foreign HLA-peptide complex, thereby bypassing the repertoire limitations imposed by thymic selection.

TCR-based therapies have had some success in clinical trials for treating melanoma, synovial sarcoma, and multiple myeloma (Morgan et al., 2006, Science 314:126-129; Johnson et al., 2009, Blood 114:535-546; Robbins, et al., 2011, J Clin Oncol 29:917-924; and Rapaport, et al., 2015, Nat Med 21:914-921). However, no TCR-based therapies have as yet received FDA approval.

### Chimeric Antigen Receptor (CAR) Therapy

Synthetic CARs provide antibody-like specificity to T cells having natural cytotoxic potency and activation potential. CARs comprise an antigen-binding region (a single-chain fragment of variable region (scFv)) derived from the antigen-binding domain of an antibody fused to the CD3ζ transmembrane and intracellular signaling domains from a TCR complex. Additional intracellular signaling domains such as CD28 and 4-1BB can be added for costimulatory signals, as in second- and third-generation CARs. This approach begins with identification of a suitable antibody targeting an appropriate cell surface antigen. Importantly, and unlike exogenous TCR therapy, CAR recognition does not rely on peptide processing or presentation by MHC molecules. As such, all surface-expressed target molecules represent a potential CAR-triggering epitope.

T cells engineered with second generation CARs having CD28 or 4-1BB signaling moieties have demonstrated potent antitumor activity in clinical trials, significantly outperforming first generation CARs. Third generation CARs incorporating another costimulatory domain are being developed to further potentiate the CAR T-cells' persistence and activity in cancer patients.

Specifically, CAR T cell therapies have had success in clinical trials for the treatment of patients with hematologic malignancies (Neelapu et al., 2017, N Engl J Med 377:2531-2544; Maude et al., N Engl J Med 378:439-448; Davila et al., 2014, Sci Trans/Med 6:224ra25; Maude et al., 2018, N Engl J Med 371:1507-1517; Kochenderfer, et al., 2015, J Clin Oncol 33:540-549; Porter et al., 2015, Sci Transl Med 7:303ra139; Turtle et al., 2017, J Clin Oncol 35:3010-3020; and Brudno et al., 2018, J Clin Oncol 36(22):2267-2280). Currently, the U.S. FDA has approved two CAR T-cell therapies: axicabtagene ciloleucel/Yescarta® for adult patients with certain types of lymphoma and tisagenlecleucel/Kymriah® for children and young adults with acute lymphoblastic leukemia (ALL) and aggressive non-Hodgkin lymphoma (NHL) who haven't responded to other forms of treatment and for adults with relapsed or refractory large B-cell lymphoma.

To date, CAR-T cell therapy against solid tumors has had limited success. Potential reasons for this include (i) inefficient T cell localization to the tumor site, (ii) physical barriers preventing tumor infiltration by T cells, (iii) increased antigen selection difficulty due to the high antigen heterogeneity of solid tumors, (iv) high risk of on-target, off-tumor toxicity due to the increased potential of target antigen expression in healthy essential organs, and (v) potent immunosuppressive factors that render T cells dysfunctional in the tumor microenvironment.

Although existing ACT results are encouraging, only a small percentage of patients with advanced malignancies can benefit from ACT thus far. Besides availability and accessibility issues for ACT, treatment-related toxicities represent a major hurdle in its widespread implementation. Thus, there is a need to develop new adoptive cell therapy cells, compositions, and methods which improve efficacy of existing ACT and/or provide enhanced efficacy of existing ACT at lower toxicity and lower costs. Accordingly, among the objects herein, it is an object herein to provide such cells, compositions, and methods.

### BRIEF SUMMARY

The present disclosure generally relates to an adoptive cell therapy method for treating a subject having a cancer or a precancer and/or for treating a subject at increased risk of developing cancer, e.g., because of a genetic risk factor or an earlier cancer or aberrant expression of at least one biomarker correlated to cancer. The method may comprise administering to the subject an effective amount of cells to the subject, wherein the cells may be modified ex vivo to suppress endogenous Zbtb20 expression and/or activity within the modified cells.

In some exemplary embodiments methods of inhibiting Zbtb20 expression and/or activity are provided, wherein such method prevents or inhibits PD-1 upregulation, and wherein Zbtb20 expression inhibition and/or activity is optionally effected by administering an effective amount of cells to the subject, wherein the cells are modified ex vivo to suppress endogenous Zbtb20 expression and/or activity within the modified cells, further these methods are optionally effected in order to prevent or inhibit T cell exhaustion in adoptive immunotherapy, further optionally adoptive immunotherapy for the treatment of cancer or an infectious condition.

In exemplary embodiments, said cells may comprise immune cells, optionally wherein said immune cells comprise T cells or T cell progenitors, preferably CD8⁺ T cells. In exemplary embodiments, the modified cells may be modified ex vivo to suppress Zbtb20 expression and/or activity. In some exemplary embodiments, said cells may further comprise at least one exogenous TCR suitable for treating cancer or at least one CAR suitable for treating cancer. In some exemplary embodiments, the method may further comprise administering one or more additional cancer therapies to the subject such as checkpoint inhibitor antibodies. In exemplary embodiments, the subject may be a mammal selected from a rodent, a non-human primate, and a human.

In some embodiments, the modified cells may be mammalian cells selected from rodent cells, non-human primate cells, and human cells. In exemplary embodiments, the cells may comprise immune cells. In some embodiments, the modified cells may comprise autologous immune cells. In exemplary embodiments, the modified cells may comprise allogenic immune cells, e.g., allogeneic T cells which optionally are modified to impair or eliminate expression of their endogenous TCR. In some embodiments, the modified cells may comprise T cells and/or T cell progenitors such as CD8⁺ T cells and/or CD4⁺ T cells. In some embodiments, the immune cells may comprise lymphocytes, T cells, NK cells, B cells, neutrophils (granulocytes), monocytes, and/or dendritic cells.

In some exemplary embodiments, the modified cells may comprise a dominant negative Zbtb20. The dominant negative Zbtb20 may comprise one or more Zbtb20 C-terminal zinc-finger domains and may lack at least a portion of a Zbtb20 N-terminal region comprising a Zbtb20 BTB domain. The dominant negative Zbtb20 may suppress endogenous Zbtb20 activity within the modified cells. In exemplary embodiments, the dominant negative Zbtb20 may comprise an amino acid sequence which is at least 75% identical, at least 80% identical, at least 85% identical, at least 90% identical, at least 95% identical, at least 98% identical, or at least 99% identical to SEQ ID NO: 40 or SEQ ID NO: 42 or to another mammalian Zbtb20 amino acid sequence. In some exemplary embodiments, the dominant negative Zbtb20 may be delivered to the modified cells prior to administering the cells to a subject. In some exemplary embodiments, the modified cells may comprise a nucleic acid encoding the dominant negative Zbtb20. Said nucleic acid may comprise a nucleotide sequence which is at least 75% identical, at least 80% identical, at least 85% identical, at least 90% identical, at least 95% identical, at least 98% identical, or at least 99% identical to SEQ ID NO: 39 or SEQ ID NO: 41 or to another mammalian Zbtb20 nucleic acid coding sequence. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some exemplary embodiments, the nucleic acid encoding the dominant negative Zbtb20 may be delivered to the modified cells prior to administering the cells to a subject. In some exemplary embodiments, the nucleic acid may be in vitro transcribed mRNA encoding the dominant negative Zbtb20. Said in vitro transcribed mRNA may be delivered to the modified cells prior to administering the cells to a subject. In some exemplary embodiments, the modified cells may be genetically engineered to express a dominant negative Zbtb20. The genetic engineering may comprise a CRISPR/Cas-based genetic engineering method, a TALEN-based genetic engineering method, a zinc finger (ZF)-nuclease genetic engineering method, or a transposon-based genetic engineering method.

In some exemplary embodiments, the modified cells may comprise at least one short hairpin RNA (shRNA) capable of suppressing endogenous Zbtb20 expression in the modified cells. In some embodiments, the at least one shRNA may be selected from SEQ ID NO: 6, SEQ ID NO: 8, SEQ ID NO: 10, SEQ ID NO: 12, SEQ ID NO: 14, and SEQ ID NO: 16. In some exemplary embodiments, the at least one shRNA may be delivered to the modified cells prior to administering the cells to a subject.

In some exemplary embodiments, the modified cells may comprise a nucleic acid encoding at least one shRNA capable of suppressing endogenous Zbtb20 expression in the modified cells. In some embodiments, said nucleic acid may comprise a nucleotide sequence selected from SEQ ID NO: 5, SEQ ID NO: 7, SEQ ID NO: 9, SEQ ID NO: 11, SEQ ID NO: 13, and SEQ ID NO: 15. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some exemplary embodiments, the nucleic acid encoding the at least one shRNA may be delivered to the modified cells prior to administering the cells to a subject.

In some exemplary embodiments, the modified cells may comprise at least one single guide RNA (sgRNA) capable of suppressing endogenous Zbtb20 expression in the modified cells. In some embodiments, said sgRNA may target at least a portion of the Zbtb20 gene. In some embodiments, said sgRNA may be selected from SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32. In exemplary embodiments, the modified cells may further comprise a protein capable of binding to the sgRNA and to at least one Zbtb20 gene portion. Said protein may be further capable of cleaving at least one DNA strand of the Zbtb20 gene portion. In exemplary embodiments, the protein is selected from a Cas9 and a Cpf1 (Cas12a). In some exemplary embodiments, the at least one sgRNA and said protein may be delivered to the modified cells prior to administering the cells to a subject.

In some exemplary embodiments, the modified cells may comprise a nucleic acid encoding at least one sgRNA capable of suppressing endogenous Zbtb20 expression in the modified cells. In some embodiments, said nucleic acid may comprise a nucleotide sequence selected from SEQ ID NO: 17, SEQ ID NO: 19, SEQ ID NO: 21, SEQ ID NO: 23, SEQ ID NO: 25, SEQ ID NO: 27, SEQ ID NO: 29, and SEQ ID NO: 31. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the modified cells may further comprise a nucleic acid encoding a protein capable of binding to the sgRNA and to at least one Zbtb20 gene portion. Said protein may be further capable of cleaving at least one DNA strand of the Zbtb20 gene portion. In exemplary embodiments, the protein is selected from a Cas9 and a Cpf1 (Cas12a). In some embodiments, the nucleic acid encoding said protein may be a construct comprising at least one promoter operatively linked to a nucleotide sequence encoding said protein. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the nucleic acid encoding said protein may be an in vitro transcribed mRNA. In some embodiments, the nucleic acid encoding the at least one sgRNA and the nucleic acid encoding said protein may be the same nucleic acid. In some embodiments, the nucleic acid encoding the at least one sgRNA and the nucleic acid encoding said protein may be separate nucleic acids. In some exemplary embodiments, the nucleic acid encoding the at least one sgRNA and the nucleic acid encoding said protein may be delivered to the modified cells prior to administering the cells to a subject.

In some exemplary embodiments, the modified cells may comprise at least one sgRNA capable of suppressing endogenous Zbtb20 expression in the modified cells. In some embodiments, said sgRNA may target a Zbtb20 promoter portion. Said Zbtb20 promoter portion may comprise DNA sequences within, encompassing, and/or close to a Zbtb20 promoter. In some embodiments, said sgRNA may be selected from SEQ ID NO: 34, SEQ ID NO: 36, and SEQ ID NO: 38. In exemplary embodiments, the modified cells may further comprise a protein capable of binding to the sgRNA and to at least one Zbtb20 promoter portion. Said Zbtb20 promoter portion may comprise DNA sequences within, encompassing, and/or close to a Zbtb20 promoter. In exemplary embodiments, the protein is selected from a Cas9 and a Cpf1 (Cas12a). In some exemplary embodiments, the at least one sgRNA and said protein may be delivered to the modified cells prior to administering the cells to a subject.

In some exemplary embodiments, the modified cells may comprise a nucleic acid encoding at least one sgRNA capable of suppressing endogenous Zbtb20 expression in the modified cells. In some embodiments, said nucleic acid may comprise a nucleotide sequence selected from SEQ ID NO: 33, SEQ ID NO: 35, and SEQ ID NO: 37. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the modified cells may further comprise a nucleic acid encoding a protein capable of binding to the sgRNA and to at least one Zbtb20 promoter portion. The Zbtb20 promoter portion may comprise DNA sequences within, encompassing, and/or close to a Zbtb20 promoter. In exemplary embodiments, the protein is selected from a Cas9 and a Cpf1 (Cas12a). In some embodiments, the nucleic acid encoding said protein may be a construct comprising at least one promoter operatively linked to a nucleotide sequence encoding said protein. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the nucleic acid encoding said protein may be an in vitro transcribed mRNA. In some embodiments, the nucleic acid encoding the at least one sgRNA and the nucleic acid encoding said protein may be the same nucleic acid. In some embodiments, the nucleic acid encoding the at least one sgRNA and the nucleic acid encoding said protein may be separate nucleic acids. In some exemplary embodiments, the nucleic acid encoding the at least one sgRNA and the nucleic acid encoding said protein may be delivered to the modified cells prior to administering the cells to a subject.

In some exemplary embodiments, the modified cells may further comprise at least one exogenous TCR suitable for treating cancer. In some embodiments, the modified cells may comprise a nucleic acid encoding the exogenous TCR suitable for treating cancer. In some exemplary embodiments, the exogenous TCR suitable for treating cancer or said nucleic acid may be delivered to the modified cells prior to administering the cells to a subject. In some embodiments, the nucleic acid encoding said exogenous TCR may be a construct comprising at least one promoter operatively linked to a nucleotide sequence encoding said exogenous TCR. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, in vitro transcribed mRNA encoding the exogenous TCR suitable for treating cancer may be delivered to the modified cells prior to administering the cells to a subject. In some embodiments, the modified cells may be genetically engineered to express the exogenous TCR suitable for treating cancer. In some embodiments, the genetic engineering may comprise a CRISPR/Cas-based genetic engineering method, a TALEN-based genetic engineering method, a ZF-nuclease genetic engineering method, or a transposon-based genetic engineering method.

In some exemplary embodiments, the modified cells may further comprise at least one CAR suitable for treating cancer. In some embodiments, the modified cells may comprise a nucleic acid encoding said CAR suitable for treating cancer. In some embodiments, the CAR suitable for treating cancer or said nucleic acid may be delivered to the modified cells prior to administering the cells to a subject. In some embodiments, the nucleic acid encoding said CAR may be a construct comprising at least one promoter operatively linked to a nucleotide sequence encoding said CAR. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, in vitro transcribed mRNA encoding the CAR suitable for treating cancer may be delivered to the modified cells prior to administering the cells to a subject. In some embodiments, the modified cells may be genetically engineered to express the CAR suitable for treating cancer. In some embodiments, the genetic engineering may comprise a CRISPR/Cas-based genetic engineering method, a TALEN-based genetic engineering method, a ZF-nuclease genetic engineering method, or a transposon-based genetic engineering method.

In some exemplary embodiments, the modified cells may be administered with cells which express at least one exogenous TCR suitable for treating cancer or with cells which express at least one CAR suitable for treating cancer, e.g., T or NK cells. The modified cells may be administered prior to, simultaneously with, or after administering said TCR- or CAR-expressing cells.

In further exemplary embodiments, the modified cells may be administered prior to, together with, or after one or more additional suitable cancer therapies. In exemplary embodiments, the one or more additional suitable cancer therapies may comprise immunotherapy, chemotherapy, targeted therapy, stem cell transplant, radiation, surgery, and hormone therapy. The immunotherapy may comprise one or more immune checkpoint inhibitors (e.g., negative checkpoint blockade), one or more monoclonal antibodies, one or more cancer vaccines, one or more immune system modulators, and one or more adoptive cell therapies. In some embodiments, the one or more adoptive cell therapies may be selected from CAR T-cell therapy, exogenous TCR therapy, and TIL therapy.

In exemplary embodiments, the at least one cancer may comprise solid and/or hematopoietic cancer. In further exemplary embodiments, the at least one cancer may comprise one or more of adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

Moreover, the present disclosure also generally encompasses an isolated cell which has been modified ex vivo to suppress endogenous Zbtb20 expression and/or activity within the cell, and to compositions comprising one or more said modified isolated cells. In exemplary embodiments, said modified isolated cell may be an immune cell, optionally wherein said immune cell may be a T cell or a T cell progenitor, preferably a CD8⁺ T cell. In exemplary embodiments, the cell may be modified to suppress Zbtb20 expression and/or activity. In some exemplary embodiments, said cell may further comprise at least one exogenous TCR suitable for treating cancer or at least one CAR suitable for treating cancer. In some exemplary embodiments, the composition comprising said modified cell may further comprise a pharmaceutically acceptable carrier. In exemplary embodiments, the modified isolated cell and the composition comprising said modified cell may be suitable for administering to a subject in a method for treating at least one cancer in the subject.

In some embodiments, the modified isolated cell may be a mammalian cell selected from a rodent cell, a non-human primate cell, and a human cell. In exemplary embodiments, the modified isolated cell may be an immune cell. In some embodiments, the modified isolated cell may be an autologous immune cell. In exemplary embodiments, the modified isolated cell may be an allogenic immune cell. In some embodiments, the modified isolated cell may be a T cell and/or a T cell progenitor such as a CD8⁺ T cell or a CD4⁺ T cell. In some embodiments, the modified isolated cell may be a lymphocyte, a T cell, an NK cell, a B cell, a neutrophil (granulocyte), a monocyte, or a dendritic cell.

In some exemplary embodiments, the modified isolated cell may comprise a dominant negative Zbtb20. The dominant negative Zbtb20 may comprise one or more Zbtb20 C-terminal zinc-finger domains and may lack at least a portion of a Zbtb20 N-terminal region comprising a Zbtb20 BTB domain. The dominant negative Zbtb20 may suppress endogenous Zbtb20 activity within the modified isolated cell. In exemplary embodiments, the dominant negative Zbtb20 may comprise an amino acid sequence which is at least 75% identical, at least 80% identical, at least 85% identical, at least 90% identical, at least 95% identical, at least 98% identical, or at least 99% identical to SEQ ID NO: 40 or SEQ ID NO: 42 or to another mammalian Zbtb20 amino acid sequence. In some exemplary embodiments, the dominant negative Zbtb20 may be delivered to the modified isolated cell prior to administering the modified isolated cell to a subject. In some exemplary embodiments, the modified isolated cell may comprise a nucleic acid encoding the dominant negative Zbtb20. Said nucleic acid may comprise a nucleotide sequence which is at least 75% identical, at least 80% identical, at least 85% identical, at least 90% identical, at least 95% identical, at least 98% identical, or at least 99% identical to SEQ ID NO: 39 or SEQ ID NO: 41 or to another mammalian Zbtb20 nucleic acid coding sequence. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some exemplary embodiments, the nucleic acid encoding the dominant negative Zbtb20 may be delivered to the modified isolated cell prior to administering the modified isolated cell to a subject. In some exemplary embodiments, the nucleic acid may be in vitro transcribed mRNA encoding the dominant negative Zbtb20. Said in vitro transcribed mRNA may be delivered to the modified isolated cell prior to administering the modified isolated cell to a subject. In some exemplary embodiments, the modified isolated cell may be genetically engineered to express a dominant negative Zbtb20. The genetic engineering may comprise a CRISPR/Cas-based genetic engineering method, a TALEN-based genetic engineering method, a ZF-nuclease genetic engineering method, or a transposon-based genetic engineering method.

In some exemplary embodiments, the modified isolated cell may comprise at least one short hairpin RNA (shRNA) capable of suppressing endogenous Zbtb20 expression in the modified isolated cell. In some embodiments, the at least one shRNA may be selected from SEQ ID NO: 6, SEQ ID NO: 8, SEQ ID NO: 10, SEQ ID NO: 12, SEQ ID NO: 14, and SEQ ID NO: 16. In some exemplary embodiments, the at least one shRNA may be delivered to the modified isolated cell prior to administering the modified isolated cell to a subject.

In some exemplary embodiments, the modified isolated cell may comprise a nucleic acid encoding at least one shRNA capable of suppressing endogenous Zbtb20 expression in the modified isolated cell. In some embodiments, said nucleic acid may comprise a nucleotide sequence selected from SEQ ID NO: 5, SEQ ID NO: 7, SEQ ID NO: 9, SEQ ID NO: 11, SEQ ID NO: 13, and SEQ ID NO: 15. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some exemplary embodiments, the nucleic acid encoding the at least one shRNA may be delivered to the modified isolated cell prior to administering the modified isolated cell to a subject.

In some exemplary embodiments, the modified isolated cell may comprise at least one single guide RNA (sgRNA) capable of suppressing endogenous Zbtb20 expression in the modified isolated cell. In some embodiments, said sgRNA may target at least a portion of the Zbtb20 gene. In some embodiments, said sgRNA may be selected from SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32. In exemplary embodiments, the modified isolated cell may further comprise a protein capable of binding to the sgRNA and to at least one Zbtb20 gene portion. Said protein may be further capable of cleaving at least one DNA strand of the Zbtb20 gene portion. In exemplary embodiments, the protein is selected from a Cas9 and a Cpf1 (Cas12a). In some exemplary embodiments, the at least one sgRNA and said protein may be delivered to the modified isolated cell prior to administering the modified isolated cell to a subject.

In some exemplary embodiments, the modified isolated cell may comprise a nucleic acid encoding at least one sgRNA capable of suppressing endogenous Zbtb20 expression in the modified isolated cell. In some embodiments, said nucleic acid may comprise a nucleotide sequence selected from SEQ ID NO: 17, SEQ ID NO: 19, SEQ ID NO: 21, SEQ ID NO: 23, SEQ ID NO: 25, SEQ ID NO: 27, SEQ ID NO: 29, and SEQ ID NO: 31. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the modified isolated cell may further comprise a nucleic acid encoding a protein capable of binding to the sgRNA and to at least one Zbtb20 gene portion. Said protein may be further capable of cleaving at least one DNA strand of the Zbtb20 gene portion. In exemplary embodiments, the protein is selected from a Cas9 and a Cpf1 (Cas12a). In some embodiments, the nucleic acid encoding said protein may be a construct comprising at least one promoter operatively linked to a nucleotide sequence encoding said protein. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the nucleic acid encoding said protein may be an in vitro transcribed mRNA. In some embodiments, the nucleic acid encoding the at least one sgRNA and the nucleic acid encoding said protein may be the same nucleic acid. In some embodiments, the nucleic acid encoding the at least one sgRNA and the nucleic acid encoding said protein may be separate nucleic acids. In some exemplary embodiments, the nucleic acid encoding the at least one sgRNA and the nucleic acid encoding said protein may be delivered to the modified isolated cell prior to administering the modified isolated cell to a subject.

In some exemplary embodiments, the modified isolated cell may comprise at least one sgRNA capable of suppressing endogenous Zbtb20 expression in the modified isolated cell. In some embodiments, said sgRNA may target a Zbtb20 promoter portion. Said Zbtb20 promoter portion may comprise DNA sequences within, encompassing, and/or close to a Zbtb20 promoter. In some embodiments, said sgRNA may be selected from SEQ ID NO: 34, SEQ ID NO: 36, and SEQ ID NO: 38. In exemplary embodiments, the modified isolated cell may further comprise a protein capable of binding to the sgRNA and to at least one Zbtb20 promoter portion. Said Zbtb20 promoter portion may comprise DNA sequences within, encompassing, and/or close to a Zbtb20 promoter. In exemplary embodiments, the protein is selected from a Cas9 and a Cpf1 (Cas12a). In some exemplary embodiments, the at least one sgRNA and said protein may be delivered to the modified isolated cell prior to administering the modified isolated cell to a subject.

In some exemplary embodiments, the modified isolated cell may comprise a nucleic acid encoding at least one sgRNA capable of suppressing endogenous Zbtb20 expression in the modified isolated cell. In some embodiments, said nucleic acid may comprise a nucleotide sequence selected from SEQ ID NO: 33, SEQ ID NO: 35, and SEQ ID NO: 37. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the modified isolated cell may further comprise a nucleic acid encoding a protein capable of binding to the sgRNA and to at least one Zbtb20 promoter portion. The Zbtb20 promoter portion may comprise DNA sequences within, encompassing, and/or close to a Zbtb20 promoter. In exemplary embodiments, the protein is selected from a Cas9 and a Cpf1 (Cas12a). In some embodiments, the nucleic acid encoding said protein may be a construct comprising at least one promoter operatively linked to a nucleotide sequence encoding said protein. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the nucleic acid encoding said protein may be an in vitro transcribed mRNA. In some embodiments, the nucleic acid encoding the at least one sgRNA and the nucleic acid encoding said protein may be the same nucleic acid. In some embodiments, the nucleic acid encoding the at least one sgRNA and the nucleic acid encoding said protein may be separate nucleic acids. In some exemplary embodiments, the nucleic acid encoding the at least one sgRNA and the nucleic acid encoding said protein may be delivered to the modified isolated cell prior to administering the cells to a subject.

In some exemplary embodiments, the modified isolated cell may further comprise at least one exogenous TCR suitable for treating cancer. In some embodiments, the modified isolated cell may comprise a nucleic acid encoding the exogenous TCR suitable for treating cancer. In some exemplary embodiments, the exogenous TCR suitable for treating cancer or said nucleic acid may be delivered to the modified isolated cell prior to administering the cells to a subject. In some embodiments, the nucleic acid encoding said exogenous TCR may be a construct comprising at least one promoter operatively linked to a nucleotide sequence encoding said exogenous TCR. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, in vitro transcribed mRNA encoding the exogenous TCR suitable for treating cancer may be delivered to the modified isolated cell prior to administering the cells to a subject. In some embodiments, the modified isolated cell may be genetically engineered to express the exogenous TCR suitable for treating cancer. In some embodiments, the genetic engineering may comprise a CRISPR/Cas-based genetic engineering method, a TALEN-based genetic engineering method, a ZF-nuclease genetic engineering method, or a transposon-based genetic engineering method.

In some exemplary embodiments, the modified isolated cell may further comprise at least one CAR suitable for treating cancer. In some embodiments, the modified isolated cell may comprise a nucleic acid encoding said CAR suitable for treating cancer. In some embodiments, the CAR suitable for treating cancer or said nucleic acid may be delivered to the modified isolated cell prior to administering the cells to a subject. In some embodiments, the nucleic acid encoding said CAR may be a construct comprising at least one promoter operatively linked to a nucleotide sequence encoding said CAR. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, in vitro transcribed mRNA encoding the CAR suitable for treating cancer may be delivered to the modified isolated cell prior to administering the cells to a subject. In some embodiments, the modified isolated cell may be genetically engineered to express the CAR suitable for treating cancer. In some embodiments, the genetic engineering may comprise a CRISPR/Cas-based genetic engineering method, a TALEN-based genetic engineering method, a ZF-nuclease genetic engineering method, or a transposon-based genetic engineering method.

The present disclosure also generally encompasses a dominant negative Zbtb20 and a nucleic acid encoding said dominant negative Zbtb20. In exemplary embodiments, the dominant negative Zbtb20 may comprise one or more Zbtb20 C-terminal zinc-finger domains and may lack at least a portion of a Zbtb20 N-terminal region comprising a Zbtb20 BTB domain. The dominant negative Zbtb20 may suppress endogenous Zbtb20 activity within a cell. In exemplary embodiments, the dominant negative Zbtb20 may comprise an amino acid sequence which is at least 75% identical, at least 80% identical, at least 85% identical, at least 90% identical, at least 95% identical, at least 98% identical, or at least 99% identical to SEQ ID NO: 40 or SEQ ID NO: 42 or to another mammalian Zbtb20 amino acid sequence. In exemplary embodiments, the nucleic acid encoding said dominant negative Zbtb20 may comprise a nucleotide sequence which is at least 75% identical, at least 80% identical, at least 85% identical, at least 90% identical, at least 95% identical, at least 98% identical, or at least 99% identical to SEQ ID NO: 39 or SEQ ID NO: 41. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the nucleic acid may be an in vitro transcribed mRNA.

The present disclosure also generally encompasses one or more shRNAs capable of suppressing Zbtb20 expression and one or more nucleic acids encoding said one or more shRNAs capable of suppressing Zbtb20 expression. In exemplary embodiments, said one or more shRNAs may be selected from SEQ ID NO: 6, SEQ ID NO: 8, SEQ ID NO: 10, SEQ ID NO: 12, SEQ ID NO: 14, and SEQ ID NO: 16. In exemplary embodiments, said one or more nucleic acids encoding said one or more shRNAs may comprise a nucleotide sequence selected from SEQ ID NO: 5, SEQ ID NO: 7, SEQ ID NO: 9, SEQ ID NO: 11, SEQ ID NO: 13, and SEQ ID NO: 15. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct.

The present disclosure also generally encompasses one or more sgRNAs capable of binding to at least a portion of the Zbtb20 gene and one or more nucleic acids encoding said one or more sgRNAs capable of binding to at least a portion of the Zbtb20 gene. In exemplary embodiments, said one or more sgRNAs may be selected from SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32. In exemplary embodiments, one or more nucleic acids encoding said one or more sgRNAs may comprise a nucleotide sequence selected from SEQ ID NO: 17, SEQ ID NO: 19, SEQ ID NO: 21, SEQ ID NO: 23, SEQ ID NO: 25, SEQ ID NO: 27, SEQ ID NO: 29, and SEQ ID NO: 31. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct.

### DESCRIPTION OF THE DRAWINGS

FIG. 1A presents a flow cytometry plot related to the phenotype of KO OT-I cells differentiated with IL-2 in vitro. Total splenocytes were harvested from KO OT-I mice, then activated with SIINFEKL peptide for 48 h without exogenous IL-2. Activated cells were further cultured with 100 U/mL recombinant human IL-2 for 7 days. Cultured cells were then analyzed by flow cytometry for CD62L levels (y-axis) and CD8 levels (x-axis).

FIG. 1B presents a flow cytometry plot related to the phenotype of wild type WT OT-I cells differentiated with IL-2 in vitro. Total splenocytes were harvested from WT OT-I mice, then activated with SIINFEKL peptide for 48 h without exogenous IL-2. Activated cells were further cultured with 100 U/mL recombinant human IL-2 for 7 days. Cultured cells were then analyzed by flow cytometry for CD62L levels (y-axis) and CD8 levels (x-axis).

FIG. 1C presents a flow cytometry plot related to the phenotype of KO OT-I cells differentiated with IL-15 in vitro. Total splenocytes were harvested from KO OT-I mice, then activated with SIINFEKL peptide for 48 h without exogenous IL-15. Activated cells were further cultured with 50 ug/mL recombinant mouse IL-15 for 7 days. Cultured cells were then analyzed by flow cytometry for CD62L levels (y-axis) and CD8 levels (x-axis).

FIG. 1D presents a flow cytometry plot related to the phenotype of WT OT-I cells differentiated with IL-15 in vitro. Total splenocytes were harvested from WT OT-I mice, then activated with SIINFEKL peptide for 48 h without exogenous IL-15. Activated cells were further cultured with 50 ug/mL recombinant mouse IL-15 for 7 days. Cultured cells were then analyzed by flow cytometry for CD62L levels (y-axis) and CD8 levels (x-axis).

FIG. 1E presents a composite of representative histograms for CD25 levels on OT-I cells. The darker shaded histogram represents data for KO OT-I cells cultured in IL-2 as described for FIG. 1A, the lighter shaded histogram represents data for WT OT-I cells cultured in IL-2 as described for FIG. 1B, the solid empty histogram represents data for KO OT-I cells cultured in IL-15 as described for FIG. 1C, and the dashed empty histogram represents data for WT OT-I cells cultured in IL-15 as described for FIG. 1D.

FIG. 2A-2H present data related to metabolic changes in in vitro generated effector and memory CD8⁺ T cells lacking Zbtb20. Total splenocytes were harvested from OT-I mice and GZB-cre Zbtb20-f/f OT-I (OT-I KO) mice, then activated with SIINFEKL peptide for 48 h without exogenous IL-2. Activated cells were further cultured with 100 U/ml rhIL-2 only or 50 ug/ml rmIL-15 for 7 days. Cultured cells were then analyzed using Seahorse XFe96 Analyzer. (A) Oxygen consumption profile showing mitochondrial respiration, (B) proton efflux rate profile showing glycolytic metabolism for IL-2 cultured cells from Seahorse XF Cell Mito stress test (A) and Seahorse XF Cell Glycolytic Rate Assay (B). (C) Mitochondrial respiratory capacity of IL-2 cultured cells measured by Seahorse XF Cell Mito stress test. (D) Glycolytic capacity of IL-2 cultured cells measured by Seahorse XF Cell Glycolytic Rate Assay. (E) Mitochondrial and (F) glycolytic metabolic profiles for IL-15 cultured cells from Seahorse XF Cell Mito stress test (E) and Seahorse XF Cell Glycolytic Rate Assay (F). (G) Mitochondrial respiratory capacity for IL-15 cultured cells measured by Seahorse XF Cell Mito stress test. (H) Glycolytic capacity of IL-15 cultured cells measured by Seahorse XF Cell Glycolytic Rate Assay. Each group consisted of at least four replicates and each experiment was repeated three times. Each point represents data from an individual mouse. Statistics were performed with unpaired Student's t-tests. *P<0.05, **P<0.01, ***P<0.001, ****P<0.0001. Representative data from three experiments are shown.

FIG. 3A-3E present data regarding how Zbtb20 affects mitochondrial surface area and volume in effector and memory CD8⁺ T cells. CD8⁺ T cells were cultured as described in FIG. 2A-2H, stained with anti-TOM20 antibody and DAPI, then analyzed by confocal microscopy. (A) Representative confocal image of KO OT-I T cells cultured with IL-2, (B) WT OT-I cells cultured with IL-2, (C) KO OT-I cells cultured with IL-15, (D) and WT OT-I cells cultured with IL-15. (E) Quantification of total mitochondrial surface area and volume in IL-2 or IL-15 treated groups. Quantification was determined on 3D reconstructed confocal images using Imaris software. Each point represents a single cell. Statistics were performed with unpaired Student's t-test. *P<0.05, **P<0.01, ***P<0.001, ****P<0.0001. Combined data from three experiments are shown.

FIG. 4A-F present data related to metabolic changes in the absence of Zbtb20 in effector and memory CD8⁺ T cells ex vivo. Naïve CD8⁺ T cells were harvested from CD45.1 OT-I mice (WT) or GZB-cre Zbtb20-f/f CD45.1 OT-I mice (KO). 50,000 naïve OT-I cells were retro-orbitally injected into B6 recipients, which were then retro-orbitally infected with 10⁶ CFU LM-actA-OVA 1 day later. On day 7 and day 28 post-infection, splenocytes were harvested from recipients and OT-I cells were purified by magnetic positive selection then subjected to mitochondrial and glycolytic metabolism analysis using the Seahorse XFe96 Analyzer. (A) Oxygen consumption profile measuring mitochondrial respiration, (B) proton efflux rate measuring glycolytic metabolism for OT-I cells enriched on day 7 post infection. (C) Mitochondrial and (D) glycolytic metabolic profiles for OT-I cells enriched on day 28 post infection. (E) Quantitation of mitochondrial respiration in OT-I cells purified on either day 7 or day 28 post-infection. (F) Quantitation of glycolytic metabolism in OT-I cells enriched on either day 7 or day 28 post infection. Each point represents data from an individual mouse. Statistics were performed with unpaired Student's t-test. *P<0.05, **P<0.01, ***P<0.001, ****P<0.0001. Representative data from three experiments are shown.

FIG. 5A-5F present data regarding how Zbtb20 deficiency affects CD8⁺ T cell metabolism after MHV-68 infection. Naïve CD8⁺ T cells were harvested from CD45.1 OT-I mice (WT) or GZB-cre Zbtb20-f/f CD45.1 OT-I mice (KO). Naïve OT-I cells were retro-orbitally injected into B6 recipient mice, which were then intra-nasally infected with MHV-68-OVA 1 day later. On day 14 or day 28 post-infection, splenocytes were harvested from recipient mice and OT-I cells were purified then subjected to mitochondrial and glycolytic metabolic analyses. (A) Oxygen consumption profile showing mitochondrial respiration, (B) proton efflux rate profile showing glycolytic metabolism for OT-I cells purified on day 14 post-infection (peak of CD8⁺ T cell response). (C) Mitochondrial and (D) glycolytic metabolic profiles for OT-I cells purified on day 28 post-infection (memory). Grey lines KO cells, black lines WT cells. (E) Quantitation of mitochondrial respiration in OT-I cells purified on either day 14 or day 28 post-infection. (F) Quantitation of glycolytic metabolism in OT-I cells enriched on either day 14 or day 28 post-infection. Each point represents data from an individual mouse. Statistics were performed using Student's unpaired t-test. *P<0.05, **P<0.01, ***P<0.001, ****P<0.0001.

FIG. 6A-6C present data related to Zbtb20 deficient effector and memory CD8⁺ T cells had higher intracellular ATP concentrations and greater mitochondria mass. Naïve CD8⁺ T cells were harvested from CD45.1 OT-I mice (WT) or GZB-cre Zbtb20-f/f CD45.1 OT-I mice (KO). 50,000 naïve OT-I cells were retro-orbitally injected into B6 recipients, which were then retro-orbitally infected with 10\hat{ }6 CFU LM-actA-OVA 1 day later. (A) On day 7 and day 28 post infection, splenocytes were harvested from recipients and OT-I cells were purified by magnetic positive selection then purified OT-I cells were analyzed by an ATP detection assay. On day 7 (B) and day 28 (C) post-infection, splenocytes were harvested from recipients, stained with mito-Tracker Green (MT-G) to quantify total mitochondrial mass then analyzed by flow cytometry. Representative histograms and quantification are shown. Shaded histogram WT, empty histogram Zbtb20 KO. Statistics were performed with unpaired Student's t-tests. *P<0.05, **P<0.01, ***P<0.001, ****P<0.0001. Data is representative of three experiments.

FIG. 7A-7E present data related to kinetics of Zbtb20 expression in CD8⁺ T cells in vivo. Naïve CD8⁺ T cells were purified from CD45.1 OT-I Zbtb20-GFP mice. 50,000 naïve OT-I cells were retro-orbitally transferred into CD45.2 B6 recipients, which were then retro-orbitally infected with 10\hat{ }6 CFU LM-actA-OVA 1 day later. Splenocytes were harvested from recipients and analyzed by flow cytometry. Naïve Zbtb20-GFP mice were used for the naïve time point. (A) Representative histograms for GFP expression at the times indicated after infection and (B) quantification. (C) Representative dot plot for CD44 and CD62L staining in naïve Zbtb20-GFP mice, (D) histograms showing corresponding GFP expression from each quadrant, shaded histogram B6 negative control, empty histogram Zbtb20 GFP. (E) Quantification of data shown in (D). Each point represents data from an individual mouse. Each group used at least four mice and each experiment was repeated three times. Statistics were performed with unpaired Student's t-tests. *P<0.05, **P<0.01, ***P<0.001, ****P<0.0001.

FIG. 8A-6D present data related to kinetics of Zbtb20 expression in mice after MHV-68 infection. Zbtb20-GFP reporter mice were intra-nasally infected with MHV-68. Splenocytes were harvested before infection and on day 10, 14 or 28 post infection analyzed for GFP expression in CD8⁺ cells staining with a tetramer representing the dominant epitope from MHV-68. (A) Representative flow plots showing ORF61 tetramer (P79) gating to identify MHV-68 specific polyclonal CD8⁺ T cells. (B) Representative dot plot showing CD44 and CD62L staining gated on tetramer+ CD8⁺ T cells, (C) histograms showing corresponding GFP expression from each quadrant, shaded histogram B6 negative control mouse, empty histogram Zbtb20-GFP mouse. (D) Quantification of data shown in (C). Each point represents data from an individual mouse. Each group used at least four mice and each experiment was repeated three times. Statistics were performed with Student's unpaired t-test or two-way ANOVA. *P<0.05, **P<0.01, ***P<0.001, ****P<0.0001.

FIG. 9A-G present data regarding Zbtb20 deletion promotes memory precursor CD8+ T cell differentiation during acute LM infection. Naïve CD8⁺ T cells were harvested from CD45.1 OT-I mice (WT) or GZB-cre Zbtb20-f/f CD45.1 OT-I mice (KO). 50,000 naïve OT-I T cells were retro-orbitally injected into B6 recipients, which were then retro-orbitally infected with 10\hat{ }6 CFU LM-actA-OVA 1 day later. Splenocytes were harvested from recipients on day 7 and day 14 post-infection and analyzed by flow cytometry. (A) Gating strategy. (B-G) All plots were gated on transferred OT-I cells. (B) Cell counts for transferred OT-I cells from the entire spleen of each recipient. (C) Representative dot plot showing KLRG-1 and CD127 staining to measure the percentage of memory precursor cells (KLRG-1-CD127+) and terminal effector cells (KLRG-1+CD127-). (D) Representative dot plot showing TNF-α and IFN-γ staining and quantification. (E) Representative dot plot showing IL-2 and IFN-γ staining and quantification. (F) Representative dot plot showing CD27 and CD8 staining and quantification. (G) Representative dot plot showing CXCR3 and CD8 staining and quantification. Each point represents data from an individual mouse. Each group used at least four mice and each experiment was repeated three times. Statistics were performed with unpaired Student's t-tests. *P<0.05, **P<0.01, ***P<0.001, ****P<0.0001.

FIG. 10A-10D present data related to Zbtb20 deletion changes expression of key transcription factors in CD8⁺ T cells during the acute response. Samples from the experiment described in FIG. 9A-9G were used for intranuclear staining for transcription factors on day 7 and day 14 post infection. (A-D) Representative histograms for (A) Bcl-6, (B) Blimp-1, (C) EOMES, (D) and T-bet staining and quantitation at 7 days post infection. Shaded histogram WT, empty histogram Zbtb20 KO. Each point represents data from an individual mouse. Each group comprised at least four mice and each experiment was repeated three times. Statistics were performed with Student's unpaired t-test. *P<0.05, **P<0.01, ***P<0.001, ****P<0.0001.

FIG. 11A-11F present data related to phenotype and function of memory CD8⁺ T cells in vivo in the absence of Zbtb20. Samples from the experiment described in FIG. 9A-9G were used to measure cytokine production potential and memory precursor or effector differentiation on days 28 and 60 post-infection. (A) Cell counts for transferred OT-I cells from the entire spleen of each recipient. (B) Representative dot plot showing KLRG-1 and CD127 staining and the percentage of memory precursors (MPEC; KLRG-1-CD127+) and terminal effector cells (SLEC; KLRG-1+CD127-). (C) Representative dot plot showing TNF-α and IFN-γ staining and quantitation. (D) Representative dot plot showing IL-2 and IFN-γ staining and quantitation. (E) Representative dot plot showing CXCR3 and CD8 staining and quantitation. (F) Representative dot plot showing CD27 and CD8 staining and quantitation. Each point represents data from an individual mouse. Each group consisted of at least four mice and each experiment was repeated three times. Statistics were performed with Student's unpaired t-test. *P<0.05, **P<0.01, ***P<0.001, ****P<0.0001.0

FIG. 12A-12D present data regarding Zbtb20 deletion changes expression of key transcription factors in memory CD8⁺ T cells. Splenocytes from mice treated as described in FIG. 9A-9G were stained for expression of intranuclear transcription factors on day 28 post infection. (A-D) Representative histogram for (A) Bcl-6, (B) Blimp-1, (C) EOMES, and (D) T-bet staining and quantification. Shaded histogram WT, empty histogram Zbtb20 KO. Each point represents data from an individual mouse. Each group comprised at least four mice and each experiment was repeated three times. Statistics were performed using Student's unpaired t-test. *P<0.05, **P<0.01, ***P<0.001, ****P<0.0001.

FIG. 13A-13C present data related to Zbtb20 deletion changes expression of key transcription factors in effector and memory CD8⁺ T cells during MHV-68 infection. Naïve CD8⁺ T cells were harvested from Thy1.1 OT-I mice (WT) or GZB-cre Zbtb20-f/f CD45.1 OT-I mice (KO) then mixed at a 1:1 ratio. Cells were retro-orbitally injected into B6 recipients, which were then intra-nasally infected with MHV-OVA 1 day later. Splenocytes were harvested from recipients on day 14 (peak response) or day 28 post-infection (memory phase) and were used for intranuclear staining of transcription factors. (A-C) Representative histograms for (A) Bcl-6, (B) EOMES, and (C) T-bet staining and quantitation at 14 and 28 days post infection. Shaded histogram WT, empty histogram Zbtb20 KO. Each point represents data from an individual mouse. Each group used at least four mice and each experiment was repeated three times. Statistics were performed using Student's paired t-test. *P<0.05, **P<0.01, ***P<0.001, ****P<0.0001.

FIG. 14A-14B present data related to Zbtb20 deletion enhances the recall response of memory CD8⁺ T cells. Adoptive transfers of OT-I cells and infection were performed as described in FIG. 9A-FIG. 9G. On day 29 or day 81 post infection, recipient mice were challenged with 10\hat{ }6 MHV-68-OVA retro-orbitally. Splenocytes were harvested 7 days post-re-challenge for flow cytometric analysis. (A-B) Cell count for transferred OT-I cells from the entire spleen of recipients challenged on (A) D28 or (B) D80 post-infection. Each point represents data from an individual mouse. Each group comprised at least four mice and each experiment was repeated three times. Statistics were performed with Student's unpaired t-test. *P<0.05, **P<0.01, ***P<0.001, ****P<0.0001.

FIG. 14C presents data related to MHV-68-OVA challenge infection is controlled in LM immune mice that received either WT or KO OT-I cells. Experimental design was as described for FIG. 14A. LM immune mice containing either WT or KO OT-I cells were challenged with MHV-68-OVA on day 28 post-infection. Data shows MHV-68-OVA titers in the spleen in four mice per group. In all cases virus was below the limit of detection (dotted line).

FIG. 15A-15B present data related to Zbtb20-deficient memory CD8⁺ T cells provide enhanced protection against MC38 tumors. Adoptive transfers of OT-I cells and infection were performed as described in FIG. 9A-FIG. 9G. At 80 days post-infection, memory OT-I cells were purified from WT or Zbtb20 KO mice, then 10\hat{ }6 cells adoptively transferred intravenously into mice that were challenged with MC38-OVA tumor subcutaneously 4 days previously. (A) Tumor area measurements. Each line represents tumor growth in an individual mouse. (B) Time to tumor growth endpoint (100 mm²). ** p<0.01 using Student's t-test (A) or Mantel-Cox log rank test (B).

FIG. 16A-16R presents gene- and pathway-level single-cell RNA-seq KO and WT comparative data. Mice received naïve OT-I or Zbtb20-deficient OT-I cells and were then infected with LM-actA-Ova. Spleen cells were harvested during the effector response, OT-I cells purified, and CITEseq/RNAseq performed as described. (A) UMAP embeddings of merged KO and WT profiles at day 10 colored by KO and WT status. (B-C) UMAP embeddings colored by expression cluster and displaying distribution of KO and WT cells within each expression cluster. KO and WT cells per cluster are denoted in C as percentages i.e. the number of KO or WT cells divided by the total number of cells in the cluster. (D) The distribution of clusters across all KO cells examined and the distribution of clusters across all WT cells is displayed as pie charts. (E-J) UMAP embeddings displaying expression of effector and memory function genes and the cell surface protein expression of the KLRG1 and CD62L markers. (K-R) UMAP embeddings of merged KO and WT profiles colored by cell-level pathway enrichment scores for gene sets in the Hallmark and C7 pathway collections in the Molecular Signature Database (MSigDB). Activity of pathways enriched in WT cells is displayed in K-N while activity of pathways enriched in KO cells are displayed in O-R.

FIG. 17A-17C contains heatmaps of differential gene and pathway expression. (A) Heatmaps displaying a subset of the top differentially expressed genes between KO and WT with genes ordered based on the cluster with the highest enrichment and cells ordered based on cluster membership or KO/WT status. All genes displayed are significantly differentially expressed between KO and WT (p<0.1). (B) Heatmaps displaying cell-level pathway enrichment of pathways differentially expressed between KO and WT with pathways ordered based on the cluster with the highest pathway enrichment score and cells ordered based on cluster membership or KO/WT status. All pathways displayed are significantly differentially expressed between KO and WT (FDR<0.15). The average log-fold change in pathway activity between KO and WT for each pathway was computed using VAM scores and is denoted. (C) Genes differentially expressed between KO and WT cells (p<0.1) that are members of the Hallmark glycolysis, oxidative phosphorylation, and reactive oxygen species pathways are displayed in heatmaps. Genes are ordered based on pathway membership. Cells are ordered based on cluster membership or KO/WT status.

FIG. 18A-18B contains the results of adoptive T cell immunotherapy against B16 melanoma which reveals that the outcome is improved in the absence of Zbtb20. (A) Schematic of experimental design testing the ability of in vitro stimulated WT or Zbtb20 KO OT-I cells from naïve mice to protect against B16-ova challenge. (B) Tumor growth curves (left) and protection (right) following B16-ova injection and T cell transfer. ** P≤0.01 using a Mantel-Cox log rank test. LM-ActA-ova: Listeria monocytogenes encoding ovalbumin. Numbers above the X-axis in (B) refer to the proportion of mice that succumbed to the tumor.

FIG. 19A-19C contains data showing that Zbtb20 deficient CD8⁺ T cells exhibit increased infiltration into tumors, and express lower levels of PD-1. (A) Schematic of experimental design, where in vitro activated WT and Zbtb20 KO OT-I cells from naïve mice were mixed at a 1:1 ratio, then transferred into B16-ova bearing mice. WT or KO cells were distinguished using congenic markers. (B) Graph showing the percentage of the total OT-I population in the tumor that were either of KO (open circles) or WT (closed squares) origin. (C) Graph showing the mean fluorescence intensity (MFI) of PD-1 staining on either KO (open circles) or WT (closed squares) OT-I cells infiltrating the tumors.

### DETAILED DESCRIPTION

### I. Overview

Provided are methods, compositions, and cells for use in cell therapy, such as adoptive cell therapy, for the treatment of subjects with a cancer or a precancer or the treatment of subjects at increased risk of developing cancer, e.g., because of a genetic risk factor or an earlier cancer or aberrant expression of at least one biomarker correlated to cancer. The methods for treating a subject having at least one cancer or a precancer or at increased risk of developing cancer involve administering an effective amount of cells to the subject, wherein the cells are modified ex vivo to suppress endogenous Zbtb20 expression and/or activity within the modified cells. Zbtb20, also known as HOF or DPZF, belongs to an evolutionarily conserved transcription factor family named broad complex, tramtrack, bric-à-brac and zinc finger (BTB-ZF) family. The cDNA and amino acid sequences for endogenous human Zbtb20 are provided in SEQ ID NO: 1 and SEQ ID NO: 2, respectively, and the cDNA and amino acid sequences for endogenous mouse Zbtb20 are provided in SEQ ID NO: 3 and SEQ ID NO: 4, respectively.

The subject may be a mammal, preferably a human. In exemplary embodiments, the cells may be immune cells, preferably T cells and/or T cell progenitors such as CD8⁺ T cells. The T cells may be further selected for the presence or absence of one or more markers, such as CD8⁺/CD45RA⁺ (e.g., naïve CD8⁺ T cells) or CD8⁺/CD45RO⁺ (e.g., antigen-experienced CD8⁺ T cells (i.e., effector or memory T cells)). The present disclosure specifically contemplates several approaches whereby the cells may be modified ex vivo to suppress endogenous Zbtb20 expression and/or activity, including but not limited to (1) use of a dominant negative Zbtb20 capable of suppressing endogenous Zbtb20 activity in the modified cells; (2) use of at least one shRNA capable of suppressing endogenous Zbtb20 expression in the modified cells; and (3) use of at least one sgRNA capable of suppressing endogenous Zbtb20 expression in the modified cells. The cells may further comprise an exogenous TCR and/or CAR suitable for treating cancer. The method may further comprise administering one or more additional cancer therapies to the subject. For example, in exemplary embodiments, the modified cells may be administered prior to, simultaneously with, or after administering cells which express at least one exogenous TCR and/or CAR suitable for treating cancer.

The present disclosure further generally relates to an isolated cell, wherein the cell is modified ex vivo to suppress endogenous Zbtb20 expression and/or activity within the cell, and to compositions comprising said modified isolated cell. In exemplary embodiments, the modified isolated cell may be an immune cell, preferably a T cell or T cell progenitor such as a CD8⁺ T cell. The modified isolated cell may be a mammalian cell, preferably a human cell. The present disclosure specifically contemplates several approaches whereby the isolated cell may be modified ex vivo to suppress endogenous Zbtb20 expression and/or activity, including but not limited to (1) use of a dominant negative Zbtb20 capable of suppressing endogenous Zbtb20 activity in the modified isolated cell; (2) use of at least one shRNA capable of suppressing endogenous Zbtb20 expression in the modified isolated cell; and (3) use of at least one sgRNA capable of suppressing endogenous Zbtb20 expression in the modified isolated cell. The modified isolated cell may further comprise an exogenous TCR and/or CAR suitable for treating cancer.

The present disclosure also provides a dominant negative Zbtb20 capable of suppressing endogenous Zbtb20 activity and to a nucleic acid encoding said dominant negative Zbtb20. Also provided herein are shRNAs and sgRNAs capable of suppressing endogenous Zbtb20 expression and nucleic acids expressing said shRNAs and sgRNAs.

It is contemplated that any embodiment discussed in this specification can be implemented with respect to any method, kit, reagent, or composition of the disclosure, and vice versa. Furthermore, compositions of this disclosure can be used to achieve methods of the disclosure.

It will be understood that particular embodiments described herein are shown by way of illustration and not as limitations. The principal features of this disclosure can be employed in various embodiments without departing from the scope of the disclosure. Those skilled in the art will recognize, or be able to ascertain, using no more than routine experimentation, numerous equivalents to the specific procedures described herein. Such equivalents are considered to be within the scope of this disclosure and are covered by the appended claims.

All publications and patent applications mentioned in the instant specification are indicative of the level of skill of one skilled in the art to which this disclosure pertains. All publications and patent applications are herein incorporated by reference to the same extent as if each individual publication or patent application was specifically and individually indicated to be incorporated by reference.

Unless defined otherwise, all technical and scientific terms used herein have the same meaning as is commonly understood by one of skill in the art to which this disclosure belongs. In the event that there are a plurality of definitions for terms herein, those in this section prevail. Where reference is made to a URL or other such identifier or address, it is to be understood that such identifiers can change and particular information on the internet can come and go, but equivalent information can be found by searching the internet. Reference thereto evidences the availability and public dissemination of such information.

As used herein, the singular forms “a,” “an,” and “the” may mean “one” but also include plural referents such as “one or more” and “at least one” unless the context clearly dictates otherwise. All technical and scientific terms used herein have the same meaning as commonly understood to one of ordinary skill in the art to which this invention belongs unless clearly indicated otherwise.

As used herein, the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 8, 9, 10, 11, 12, 13, 14, or 15%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result alone or in combination with other active agents.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts alone or in combination with other active agents or therapies, e.g., those used in cancer treatment.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

As used herein, “Zbtb20” and other forms thereof (including “zbtb20” and “ZBTB20”) refers to “zinc finger and BTB domain containing 20” protein, transcript (mRNA), and/or gene expressing said protein from human (NCBI GeneID No. 26137), mouse (NCBI GeneID No. 56490), or from any other mammalian species, including all isoforms thereof. Zbtb20 is also known as DPZF, HOF, ODA-8S, PRIMS, and ZNF288. Zbtb20 may have a cDNA nucleotide sequence which is at least 75% identical, at least 80% identical, at least 85% identical, at least 90% identical, at least 95% identical, at least 98% identical, at least 99% identical or more to SEQ ID NO: 1 or SEQ ID NO: 3 or to any other mammalian Zbtb20 cDNA sequence. Zbtb20 may have an amino sequence which is at least 75% identical, at least 80% identical, at least 85% identical, at least 90% identical, at least 95% identical, at least 98% identical, at least 99% identical or more to SEQ ID NO: 2 or SEQ ID NO: 4 or to any other mammalian Zbtb20 amino acid sequence.

As used herein, “modified to suppress endogenous Zbtb20 expression and/or activity” refers to any type of modification which specifically reduces the expression level of the endogenous Zbtb20 gene and/or mRNA and/or protein compared to the expression level of said gene and/or mRNA and/or protein when said modification is not present, or to any type of modification which specifically reduces the level of any activity of endogenous Zbtb20 compared to the level of said activity when said modification is not present. The modification may lead to a reduction of the expression level of the endogenous Zbtb20 gene and/or mRNA and/or protein by at least 10%, at least 20%, at least 30%, at least 40%, at least 50%, at least 60%, at least 70%, at least 80%, at least 90%, at least 99%, or more. The modification may lead to a reduction of the level of any activity of endogenous Zbtb20 by at least 10%, at least 20%, at least 30%, at least 40%, at least 50%, at least 60%, at least 70%, at least 80%, at least 90%, at least 99%, or more. The modification may be a permanent modification or a temporary modification.

As used herein, “dominant negative Zbtb20” refers to any variant of endogenous Zbtb20 which is capable of suppressing the activity of endogenous Zbtb20. The dominant negative Zbtb20 may act as a competitive inhibitor of Zbtb20, whereby the dominant negative Zbtb20 binds to endogenous Zbtb20 binding sites within DNA and thereby prevents the binding of endogenous Zbtb20 to said binding sites. It is contemplated that the dominant negative Zbtb20 comprises one or more Zbtb20 C-terminal zinc-finger domains and lacks at least a portion of a Zbtb20 N-terminal region comprising a Zbtb20 BTB domain.

As used herein, “capable of suppressing endogenous Zbtb20 expression” refers to an ability of any factor, such as shRNA or sgRNA, to specifically reduce the expression level of the endogenous Zbtb20 gene and/or mRNA and/or protein compared to the expression level of said gene and/or mRNA and/or protein when said factor is not present. Said factor may independently posses said ability or may require additional factors which may or may not be recited herein. As such, said factor may contribute to the specific reduction of the expression level of the endogenous Zbtb20 gene and/or mRNA and/or protein compared to said expression level when said factor is not present. For example, “shRNA capable of suppressing endogenous Zbtb20 expression” refers herein to shRNA which may require additional factors such as endogenous Drosha, Dicer, and RISC to be capable of suppressing endogenous Zbtb20 expression (see, e.g., Wilson and Doudna, 2013, Annu. Rev. Biophys. 42:217-39). Further, “sgRNA capable of suppressing endogenous Zbtb20 expression” refers herein to sgRNA which may require additional factors such as a Cas9 or a Cpf1 (Cas12a) to be capable of suppressing endogenous Zbtb20 expression (see, e.g., Knott and Doudna, 2018, Science, 361(6405):866-869.

As used herein, “cancer” refers to any disease in which abnormal cells divide without control and which can invade nearby tissues or spread to other parts of the body through the blood and lymph systems. Cancer may include carcinomas (cancers that begin in the skin or in tissues that line or cover internal organs), sarcomas (cancers that begin in bone, cartilage, fat, muscle, blood vessels, or other connective or supportive tissue), leukemias (cancers that start in blood-forming tissue, such as the bone marrow, and causes large numbers of abnormal blood cells to be produced and enter the blood), lymphomas and multiple myelomas (cancers that begin in the cells of the immune system), and central nervous system cancers (cancers that begin in the tissues of the brain and spinal cord). Cancer may also refer to any malignancy. Types of cancer include but are not limited to adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

The term “autologous” refers to any material derived from the same individual to whom it is later to be re-introduced.

The term “allogenic” refers to any material derived from a different animal of the same species as the individual to whom the material is to be introduced or transplanted. Two or more individuals are said to be allogeneic to one another when the genes at one or more loci are not identical. In some aspects, allogeneic material from individuals of the same species may be sufficiently dissimilar genetically to interact antigenically.

II. Modified Cells Suppressing Endogenous Zbtb20 Expression and/or Activity

### A. Cells

The cells generally are eukaryotic cells, such as mammalian cells, and typically are human cells, e.g., those derived from human subjects and modified, for example, to suppress endogenous Zbtb20 expression and/or activity. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells, NK cells, or B cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD8⁺ cells, CD4⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some embodiments, the methods include isolating cells from the subject, preparing, processing, culturing, and/or engineering them, and re-introducing them into the same subject, before or after cryopreservation of the cells.

Among the sub-types and subpopulations of T cells and/or of CD8⁺ and/or of CD4⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are B cells or natural killer (NK) cells. In some embodiments, the cells are monocytes or granulocytes, e.g., myeloid cells, macrophages, neutrophils, dendritic cells, mast cells, eosinophils, and/or basophils.

### B. Dominant Negative Zbtbt20 for Suppressing Endogenous Zbtb20 Activity

In one group of embodiments, the method may involve administering an effective amount of cells comprising a dominant negative Zbtb20 which suppresses endogenous Zbtb20 activity. The dominant negative Zbtb20 may comprise one or more Zbtb20 C-terminal zinc-finger domains and may lack at least a portion of a Zbtb20 N-terminal region comprising a Zbtb20 BTB domain. The dominant negative Zbtb20 may suppress endogenous Zbtb20 activity within the modified cells, for example, by binding to Zbtb20 binding sites within DNA thereby preventing endogenous Zbtb20 from binding to said DNA sites. In exemplary embodiments, the dominant negative Zbtb20 may comprise an amino acid sequence which is at least 75% identical, at least 80% identical, at least 85% identical, at least 90% identical, at least 95% identical, at least 98% identical, or at least 99% identical to SEQ ID NO: 40 or SEQ ID NO: 42. In some exemplary embodiments, the dominant negative Zbtb20 may be delivered to the modified cells prior to administering the cells to a subject. As discussed below, methods for delivering proteins to mammalian cells are known in the art.

In some exemplary embodiments, the modified cells may comprise a nucleic acid encoding the dominant negative Zbtb20. Said nucleic acid may comprise a nucleotide sequence which is at least 75% identical, at least 80% identical, at least 85% identical, at least 90% identical, at least 95% identical, at least 98% identical, or at least 99% identical to SEQ ID NO: 39 or SEQ ID NO: 41. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some exemplary embodiments, the nucleic acid encoding the dominant negative Zbtb20 may be delivered to the modified cells prior to administering the cells to a subject. In some exemplary embodiments, the nucleic acid may be in vitro transcribed mRNA encoding the dominant negative Zbtb20. Said in vitro transcribed mRNA may be delivered to the modified cells prior to administering the cells to a subject. In some exemplary embodiments, the modified cells may be genetically engineered to express a dominant negative Zbtb20. The genetic engineering may comprise a CRISPR/Cas-based genetic engineering method, a TALEN-based genetic engineering method, a ZF-nuclease genetic engineering method, or a transposon-based genetic engineering method. As discussed below, methods for delivering nucleic acids (plasmids, constructs, and mRNAs) to mammalian cells and for genetically engineering mammalian cells are known in the art.

C. Short Hairpin RNA (shRNA) for Suppressing Endogenous Zbtb20 Expression

In one group of embodiments, the method may involve administering an effective amount of cells comprising at least one shRNA capable of suppressing endogenous Zbtb20 expression in the modified cells. In some embodiments, the at least one shRNA may be selected from SEQ ID NO: 6, SEQ ID NO: 8, SEQ ID NO: 10, SEQ ID NO: 12, SEQ ID NO: 14, and SEQ ID NO: 16. In some exemplary embodiments, the at least one shRNA may be delivered to the modified cells prior to administering the cells to a subject. As discussed below, methods for delivering nucleic acids, including shRNA, to mammalian cells are known in the art.

In some exemplary embodiments, the modified cells may comprise a nucleic acid encoding at least one shRNA capable of suppressing endogenous Zbtb20 expression in the modified cells. In some embodiments, said nucleic acid may comprise a nucleotide sequence selected from SEQ ID NO: 5, SEQ ID NO: 7, SEQ ID NO: 9, SEQ ID NO: 11, SEQ ID NO: 13, and SEQ ID NO: 15. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some exemplary embodiments, the nucleic acid encoding the at least one shRNA may be delivered to the modified cells prior to administering the cells to a subject. As discussed below, methods for delivering nucleic acids, such as plasmids and constructs, to mammalian cells are known in the art.

D. Single Guide RNA (sgRNA) for Suppressing Endogenous Zbtb20 Expression

In one group of embodiments, the method may involve administering an effective amount of cells comprising at least one sgRNA capable of suppressing endogenous Zbtb20 expression in the modified cells. In some embodiments, said sgRNA may target at least a portion of the Zbtb20 gene. In some embodiments, said sgRNA may be selected from SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32. In exemplary embodiments, the modified cells may further comprise a protein capable of binding to the sgRNA and to at least one Zbtb20 gene portion. Said protein may be further capable of cleaving at least one DNA strand of the Zbtb20 gene portion. In exemplary embodiments, the protein is selected from a Cas9 and a Cpf1 (Cas12a). In some exemplary embodiments, the at least one sgRNA and said protein may be delivered to the modified cells prior to administering the cells to a subject, either separately or together as a ribonucleoprotein complex. As discussed below, methods for delivering nucleic acids, including sgRNA, proteins, and ribonucleoprotein complexes to mammalian cells are known in the art.

In some exemplary embodiments, the modified cells may comprise a nucleic acid encoding at least one sgRNA capable of suppressing endogenous Zbtb20 expression in the modified cells. In some embodiments, said nucleic acid may comprise a nucleotide sequence selected from SEQ ID NO: 17, SEQ ID NO: 19, SEQ ID NO: 21, SEQ ID NO: 23, SEQ ID NO: 25, SEQ ID NO: 27, SEQ ID NO: 29, and SEQ ID NO: 31. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. As discussed below, methods for delivering nucleic acids, such as plasmids and constructs, to mammalian cells are known in the art. In some embodiments, the modified cells may further comprise a nucleic acid encoding a protein capable of binding to the sgRNA and to at least one Zbtb20 gene portion. Said protein may be further capable of cleaving at least one DNA strand of the Zbtb20 gene portion. In exemplary embodiments, the protein is selected from a Cas9 and a Cpf1 (Cas12a). In some embodiments, the nucleic acid encoding said protein may be a construct comprising at least one promoter operatively linked to a nucleotide sequence encoding said protein. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the nucleic acid encoding said protein may be an in vitro transcribed mRNA. In some embodiments, the nucleic acid encoding the at least one sgRNA and the nucleic acid encoding said protein may be the same nucleic acid. In some embodiments, the nucleic acid encoding the at least one sgRNA and the nucleic acid encoding said protein may be separate nucleic acids. In some exemplary embodiments, the nucleic acid encoding the at least one sgRNA and the nucleic acid encoding said protein may be delivered to the modified cells prior to administering the cells to a subject. As discussed below, methods for delivering nucleic acids, such as plasmids and constructs, to mammalian cells are known in the art.

In one group of embodiments, the method may involve administering an effective amount of cells comprising at least one sgRNA capable of suppressing endogenous Zbtb20 expression in the modified cells. In some embodiments, said sgRNA may target a Zbtb20 promoter portion. Said Zbtb20 promoter portion may comprise DNA sequences within, encompassing, and/or close to a Zbtb20 promoter. In some embodiments, said sgRNA may be selected from SEQ ID NO: 34, SEQ ID NO: 36, and SEQ ID NO: 38. In exemplary embodiments, the modified cells may further comprise a protein capable of binding to the sgRNA and to at least one Zbtb20 promoter portion. Said Zbtb20 promoter portion may comprise DNA sequences within, encompassing, and/or close to a Zbtb20 promoter. In exemplary embodiments, the protein is selected from a Cas9 and a Cpf1 (Cas12a). In some exemplary embodiments, the at least one sgRNA and said protein may be delivered to the modified cells prior to administering the cells to a subject, either separately or together as a ribonucleoprotein complex. As discussed below, methods for delivering nucleic acids, including sgRNA, proteins, and ribonucleoprotein complexes to mammalian cells are known in the art.

In some exemplary embodiments, the modified cells may comprise a nucleic acid encoding at least one sgRNA capable of suppressing endogenous Zbtb20 expression in the modified cells. In some embodiments, said nucleic acid may comprise a nucleotide sequence selected from SEQ ID NO: 33, SEQ ID NO: 35, and SEQ ID NO: 37. In some embodiments, the nucleic acid may be a construct comprising at least one promoter operatively linked to said nucleotide sequence. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the modified cells may further comprise a nucleic acid encoding a protein capable of binding to the sgRNA and to at least one Zbtb20 promoter portion. The Zbtb20 promoter portion may comprise DNA sequences within, encompassing, and/or close to a Zbtb20 promoter. In exemplary embodiments, the protein is selected from a Cas9 and a Cpf1 (Cas12a). In some embodiments, the nucleic acid encoding said protein may be a construct comprising at least one promoter operatively linked to a nucleotide sequence encoding said protein. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, the nucleic acid encoding said protein may be an in vitro transcribed mRNA. In some embodiments, the nucleic acid encoding the at least one sgRNA and the nucleic acid encoding said protein may be the same nucleic acid. In some embodiments, the nucleic acid encoding the at least one sgRNA and the nucleic acid encoding said protein may be separate nucleic acids. In some exemplary embodiments, the nucleic acid encoding the at least one sgRNA and the nucleic acid encoding said protein may be delivered to the modified cells prior to administering the cells to a subject. As discussed below, methods for delivering nucleic acids, such as plasmids and constructs, to mammalian cells are known in the art.

### E. Recombinant Antigen Receptors

In some embodiments, the modified cells may be further modified to comprise recombinant antigen receptors, or the modified cells may be administered in combination with other cells which comprise recombinant antigen receptors. The antigen receptors may include exogenous TCRs and chimeric antigen receptors (CARs), as well as other chimeric receptors, such as receptors binding to particular ligands and having transmembrane and/or intracellular signaling domains similar to those present in a CAR. In some embodiments, the modified cells may comprise a nucleic acid encoding the exogenous TCR or CAR suitable for treating cancer. In some exemplary embodiments, the exogenous TCR or CAR suitable for treating cancer or said nucleic acid may be delivered to the modified cells prior to administering the cells to a subject. In some embodiments, the nucleic acid encoding said exogenous TCR or CAR may be a construct comprising at least one promoter operatively linked to a nucleotide sequence encoding said exogenous TCR or CAR. The promoter may be a constitutive promoter or an inducible promoter. In exemplary embodiments, the construct may be selected from a plasmid, a retrovirus construct, a lentivirus construct, an adenovirus construct, and an adeno-associated virus (AAV) construct. In some embodiments, in vitro transcribed mRNA encoding the exogenous TCR or CAR suitable for treating cancer may be delivered to the modified cells prior to administering the cells to a subject. In some embodiments, the modified cells may be genetically engineered to express the exogenous TCR or CAR suitable for treating cancer. In some embodiments, the genetic engineering may comprise a CRISPR/Cas-based genetic engineering method, a TALEN-based genetic engineering method, a ZF-nuclease genetic engineering method, or a transposon-based genetic engineering method. As discussed below, methods for delivering proteins and nucleic acids (plasmids, constructs, and mRNAs) to mammalian cells and for genetically engineering mammalian cells are known in the art.

In further exemplary embodiments, the modified cells may be administered with cells which express at least one exogenous TCR suitable for treating cancer or with cells which express at least one CAR suitable for treating cancer. The modified cells may be administered prior to, simultaneously with, or after administering said TCR- or CAR-expressing cells.

Exemplary antigen receptors and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Morgan et al., 2006, Science 314:126-129; Johnson et al., 2009, Blood 114:535-546; Robbins, et al., 2011, J Clin Oncol 29:917-924; Rapaport, et al., 2015, Nat Med 21:914-921; Neelapu et al., 2017, N Engl J Med 377:2531-2544; Maude et al., 2018, N Engl J Med 378:439-448; Davila et al., 2014, Sci Transl Med 6:224ra25; Maude et al., 2014, N Engl J Med 371:1507-1517; Kochenderfer, et al., 2015, J Clin Oncol 33:540-549; Porter et al., 2015, Sci Transl Med 7:303ra139; Turtle et al., 2017, J Clin Oncol 35:3010-3020; Brudno et al., 2018, J Clin Oncol 36(22):2267-2280, Sadelain et al., 2013, Cancer Discov. 3(4):388-398; Davila et al., 2013, PLoS ONE 8(4):e61338; Turtle et al., 2012, Curr. Opin. Immunol., 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276; Wang et al., 2012, J. Immunother. 35(9): 689-701; and Brentjens et al., 2013, Sci Transl Med. 2013 5(177). See also International Patent Publication No.: WO2014031687, U.S. Pat. Nos. 8,339,645, 7,446,179, 7,446,190, and 8,389,282, and U.S. patent application Publication No. US 2013/0149337.

### F. Methods for Modifying Cells

Cells of the present disclosure may be modified ex vivo by delivering certain proteins and/or nucleic acids of the disclosure to the cells, or by genetically engineering the cells. Methods for delivering proteins and nucleic acids to mammalian cells are known in the art. See, e.g., Bruce and McNaughton, 2017, Cell Chem. Biol. 24(8):924-934 and Stewart et al., (2016) Nature, 538:183-192 and references cited therein. For example, nucleic acids can be delivered to mammalian cells ex vivo by use of cationic lipids (Morille et al., 2008, Biomaterials, 29(24-25):3477-96) or by electroporation methods such as nucleofection (Maasho et al., J. Immunol. Methods, (2004) 284:133-140). Cationic lipids can also be used to deliver proteins to mammalian cells (Zuris et al., (2015), Nat. Biotechnol., 33:73-80). Additionally, methods for genetically engineering mammalian cells are also known in the art. See, e.g., Senis, et al., Biotech. J. (2014) 9(11):1402-1412; Knott and Doudna, Science (2018) 361(6405):866-869; Tipanee et al., Biosci. Rep. (2017) 37(6) BSR20160614; Yin et al., Nat. Rev. Drug Discov. (2017) 16(6):387-399; and references cited therein. Suitable genetic engineering methods may include a CRISPR/Cas-based genetic engineering method, a TALEN-based genetic engineering method, a ZF-nuclease genetic engineering method, or a transposon-based genetic engineering method. Further, in vitro transcribed mRNA may be delivered to cells ex vivo in order to express a protein of interest in the modified cells, such as a dominant negative Zbtb20. Methods for generating in vitro transcribed mRNA and delivering said mRNA are well known in the art (see, e.g., Coutinho et al., Adv. Exp. Med. Biol. (2019) 1157:133-177; US Patent Pub. 20130245106; and US Patent Pub. 20170173128).

The present disclosure provides vectors or constructs including plasmids and viral constructs suitable for expressing various factors of the disclosure in mammalian cells. A nucleotide sequence (such as one encoding a dominant negative Zbtb20, one or more shRNA(s), one or more sgRNA(s), an exogenous TCR, a CAR, or a Cas-type nuclease) may be inserted into a vector or viral construct, including those from retroviruses, lentiviruses, adenoviruses, and adeno-associated viruses (AAV). Viral vector technology is well known in the art and is described, for example, in Sambrook et al. (2001, Molecular Cloning: A Laboratory Manual, Cold Spring Harbor Laboratory, New York), and in other virology and molecular biology manuals. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. The expression of natural or synthetic nucleic acids encoding proteins, mRNA, or non-coding RNAs of interest may typically be achieved by operably linking a nucleic acid encoding said proteins, mRNA, or non-coding RNAs to a promoter, and incorporating the construct into an expression vector. The vectors can be suitable for replication or replication and integration in eukaryotes. Typical vectors contain transcription and translation terminators, initiation sequences, and promoters (either constitutive or inducible promoters) useful for regulation of the expression of the desired nucleic acid sequence. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

### III. Administration of Cells in Adoptive Cell Therapy Methods

The provided methods generally involve administering an effective amount of modified cells such as such as the cells discussed above which have been modified ex vivo to suppress endogenous Zbtb20 expression and/or activity, to subjects having at least one cancer. As discussed above, the cells may be further modified to express an exogenous TCR and/or CAR suitable for treating cancer. The administration generally effects an improvement in one or more symptoms of the cancer and/or treats or prevents the cancer or symptoms thereof.

As used herein, a “subject” is a mammal, such as a human or other animal, and typically is a human. In some embodiments, administration of the effective amount of cells is the first cancer treatment the subject has received. In some embodiments, the subject has been treated with one or more additional cancer therapies prior to the administration of the modified cells. In some aspects, the subject may be or may have become refractory or non-responsive to the other treatment. In some embodiments, the subject may not have become refractory or non-responsive but the administration of the modified cells is carried out to complement the other treatment and/or enhance the subject's response to the other treatment. In some embodiments the modified cells are administered prior to or simultaneously with the other treatment. It is contemplated by this disclosure that the other treatment comprising one or more additional cancer therapies may include immunotherapy, chemotherapy, targeted therapy, stem cell transplant, radiation, surgery, and/or hormone therapy. In some embodiments, the immunotherapy may include immune checkpoint inhibitors (e.g., negative checkpoint blockade), monoclonal antibodies, cancer vaccines, immune system modulators, and/or adoptive cell therapies such as CAR T-cell therapy, exogenous TCR therapy, and TIL therapy.

In some embodiments, the cells are administered as part of a combination treatment, such as simultaneously with or sequentially with, in any order, another therapeutic intervention, such as an antibody or engineered cell or receptor or other agent, such as a cytotoxic or therapeutic agent. Thus, the cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, IL-15, or other cytokine, for example, to enhance persistence. In some embodiments, the methods comprise administration of a chemotherapeutic agent, e.g., a conditioning chemotherapeutic agent, for example, to reduce tumor burden prior to the dose administrations.

In some embodiments, the subject may be subjected to lymphodepletion procedures prior to administration of the modified cells. In some embodiments, the subject may receive a nonmyeloablative lymphodepletion regimen or may undergo lymphodepletion with hematopoietic stem cell transplantation prior to administration of the modified cells. Methods to induce lymphopenia include treatment with low-dose total body irradiation (TBI) that produces mild, reversible myelosuppression (hence nonmyeloablative) and/or treatment with chemotherapeutic drugs such as cyclophosphamide (Cy) alone or in combination with fludarabine. Procedures for lymphodepletion are known in the art. See, e.g., Wrzesinski et al. (2007) J. Clin. Invest., 117(2):492-501.

In some embodiments the subject may receive a single dose of the modified cells. In some embodiments, the subject may receive multiple doses of the modified cells. In some embodiments, the cancer comprises a tumor and the subject has a large tumor burden prior to the administration of the first dose, such as a large solid tumor or a large number or bulk of tumor cells. In some aspects, the subject has a high number of metastases and/or widespread localization of metastases. In some aspects, the tumor burden in the subject is low and the subject has few metastases. In some embodiments, the size or timing of the doses is determined by the initial disease burden in the subject. For example, whereas in some aspects the subject may be administered a relatively low number of cells in a first dose, in the context of a higher disease burden, the dose may be higher and/or the subject may receive one or more additional doses.

Administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than seven days. Thus, in some contexts, the dose is a single or continuous administration of the specified number of cells, given or initiated at a single point in time. In some contexts, however, the dose is administered in multiple injections or infusions over a period of no more than seven days, such as once a day for three days or for two days or by multiple infusions over a single day period.

In some embodiments, for example, where the subject is a human, the dose includes fewer than about 1×10⁸ total modified cells, recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, the dose contains fewer than about 1×10⁸ total modified cells, recombinant receptor (e.g., CAR)-expressing cells, T cells, or peripheral blood mononuclear cells (PBMCs) cells per m² of the subject, e.g., in the range of about 1×10⁶ to 1×10⁸ such cells per m² of the subject, such as 2×10⁶, 5×10⁶, 1×10⁷, 5×10⁷, or 1×10⁸ such cells per m² of the subject, or the range between any two of the foregoing values. In certain embodiments, the number of modified cells, recombinant receptor (e.g., CAR)-expressing cells, T cells, or peripheral blood mononuclear cells (PBMCs) in the first or subsequent dose is greater than about 1×10⁶ such cells per kilogram body weight of the subject, e.g., 2×10⁶, 3×10⁶, 5×10⁶, 1×10⁷, 5×10⁷, 1×10⁸, 1×10⁹, or 1×10¹⁰ such cells per kilogram of body weight and/or, 1×10⁸, or 1×10⁹, 1×10¹⁰ such cells per m² of the subject or total, or the range between any two of the foregoing values.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; and in Rosenberg (2011) Nat Rev Clin Oncol. 8(10):577-85; Themeli et al. (2013) Nat Biotechnol. 31(10):928-933; Tsukahara et al. (2013) Biochem Biophys Res Commun 438(1): 84-9; Davila et al. (2013) PLoS ONE 8(4):e61338; and Wennhold et al., Transfus Med Hemother 2019; 46:36-46.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical or similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of cancer, the appropriate dosage may depend on the type of cancer to be treated, the type of modified cells, the type of recombinant receptors if present, the severity and course of the cancer, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In certain embodiments, the modified cells may be further modified in any number of ways, such that their therapeutic or prophylactic efficacy is increased. For example, the modified cells may express an endogenous cell surface receptor or may be engineered to express a cell surface receptor, such as an exogenous TCR or CAR, which can then be conjugated either directly or indirectly through a linker to a targeting moiety. The practice of conjugating compounds to targeting moieties is known in the art. See, for instance, Wadwa et al., J. Drug Targeting 3: 111 (1995), and U.S. Pat. No. 5,087,616.

Also provided are compositions including the cells, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

All of the compositions and/or methods disclosed and claimed herein can be made and executed without undue experimentation in light of the present disclosure. While the compositions and methods have been described in terms of preferred embodiments, it will be apparent to those of skill in the art that variations and substitutions may be applied to the compositions and/or methods and in the steps or in the sequence of steps of the methods described herein without departing from the concept, spirit and scope of the disclosure.

### IV. Examples

Unless stated otherwise the following Materials and Methods were used in the Examples which follow.

### Materials and Methods

Mice, virus and bacteria. Zbtb20-GFP mice (MMRRC #030006-UCD) were obtained from the Knockout Mouse Project (KOMP). Zbtb20-fl/fl mice were generated by Dr. Weiping J. Zhang (Second Military Medical University, China) (Xie, Z., H. et al., 2008, “Zinc finger protein ZBTB20 is a key repressor of alpha-fetoprotein gene transcription in liver”, Proceedings of the National Academy of Sciences of the United States of America). OT-I mice were originally purchased from Jackson Laboratory (003831). CD45.1 mice were purchased from Jackson Laboratory (002014). GZB-cre mice were kindly provided by Dr. Rafi Ahmed (Emory University). CD45.1 OT-I mice, Zbtb20-GFP CD45.1 OT-I mice and GZB-cre Zbtb20-flox CD45.1 OT-I mice were crossed and bred in-house at Dartmouth College. MHV-68-Ova virus was kindly provided by Dr. Phillip Stevenson (University of Queensland, Australia). LM-actA-Ova was kindly provided by Dr. John Harty (University of Iowa).

Primers. Primers GCAAGTTGCAGGCACAGCTAGTT and TAGCGGCTGAAGCACTGCA were used to genotype Zbtb20-GFP mice. Primers GZACCGCTGGCAACACCTATCTG and CTCTCCCCTCCTCCCTCTGG were used to genotype Zbtb20-floxed mice. Primers GCATTACCGGTCGATGCAACGAGTGATGAG and GAGTGAACGAACCTGGTCGAAATCAGTGCG were used to genotype GZB-cre mice. Primers CCTGCCTGAACTTTGAAGCTGTT and GCAACTGATGTCACAATCAGATGACC were used for ZBTB20 quantitative fluorescent PCR (QF-PCR).

IL-2/IL-15 in vitro CD8⁺ T cell differentiation. Total splenocytes were harvested from OT-I mice and GZB-cre Zbtb20-fl/fl OT-I mice, then seeded at 2×10⁶ cells/mL with 10 μg/mL SIINFEKL peptide for 48 h without exogenous IL-2. Activated cells were further cultured with 100 U/ml rhIL-2 only at 0.5×106 cells/mL or with 50 ug/ml rmIL-15 at 10⁶ cells/mL for 7 days. Cultures were split and provided fresh media every 2-3 days.

Seahorse analysis. Assays were performed according to the manufacturer's protocols. 150,000 cells were seeded per well for IL-2/IL-15 in vitro differentiated CD8⁺ T cells. 200,000 cells were seeded per well for ex vivo CD8⁺ T cells. 1 μM oligomycin, 1.5 μM FCCP and 0.5 μM R/AA were used for mitochondrial stress assays (Seahorse XF Cell Mito Stress Test Kit; Seahorse Agilent cat: 103015-100); 0.5 μM Rotenone/Antimycin A and 50 mM 2-Deoxyglucose were used for Glycolytic rate assays (Seahorse XF Glycolytic rate Assay; Seahorse Agilent cat: 103344-100).

Ex vivo Seahorse Bioanalyzer Assays. Naïve CD8⁺ T cells were harvested from CD45.1 OT-I mice (WT) or GZB-cre Zbtb20-fl/fl CD45.1 OT-I mice (KO) using EasySep mouse naïve CD8⁺ T cell isolation kits (StemCell Technologies cat: 19858A). 50,000 naïve OT-I cells were retro-orbitally injected into B6 recipients, which were then retro-orbitally infected with 10⁶ CFU LM-actA-Ova 1 day later. On D7 and D28 post infection, splenocytes were harvested from recipients, stained with anti-CD45.1-APC antibody then purified with Mojosort mouse anti-APC nanobeads (Biolegend Cat: 480072). 200,000 enriched cells (purity greater than 95%) were seeded into each well for Seahorse mitochondrial stress tests and Glycolytic Rate tests.

1 μM oligomycin, 1.5 μM 4-(trifluoromethoxy)phenyl)carbonohydrazonoyl dicyanide (FCCP) and 0.5 μM Rotenone/Antimycin A were used for mitochondria stress assays. 0.5 μM Rotenone/Antimycin A and 50 mM 2-deoxyglucose were used for Glycolytic rate assays.

Mitochondrial fuel flexibility assays. Total splenocytes were harvested from OT-I mice and GZB-cre ZBTB20-f/f OT-I (KO) mice, then activated with SIINFEKL peptide for 48 h without exogenous IL-2. Activated cells were further cultured with 50 ug/ml rmIL-15 for 7 days. Cultured cells were then analyzed using Seahorse XFe96 Analyzer. Cells were treated with no inhibitors or combinations of different inhibitors that prevented the utilization of different mitochondrial fuel source (etomoxir for long-chain fatty-acid; UK5099 for pyruvate; BPTES for L-glutamine; utilization of short and medium chain fatty acid were not manipulated), followed by a conventional Seahorse Agilent Mito Stress test. The maximal Respiratory Capacity of each condition was normalized to the group without inhibitor treatment. 4 μM Etomoxir, 2 μM UK5099, 3 mm BPTES, 1 μM oligomycin, 1.5 μM FCCP and 0.5 μM R/AA were used for mitochondrial fuel flexibility assay (Seahorse XF Cell Mito Stress Test Kit; Seahorse Agilent cat: 103015-100).

Adoptive transfers. Naïve CD8⁺ T cells were harvested from CD45.1 OT-I mice (WT) or GZB-cre Zbtb20-fl/fl CD45.1 OT-I mice (KO) and purified using EasySep mouse naïve CD8 T cell isolation kits (Stemcell Technologies cat: 19858A). 50,000 naïve OT-I cells were retro-orbitally injected into congenic B6 recipient mice, which were then retro-orbitally infected with 10⁶ CFU LM-actA-Ova 1 day later.

MC38-Ova tumor protection. Naïve CD8⁺ T cells were harvested from CD45.1 OT-I mice (WT) or GZB-cre Zbtb20-fl/fl CD45.1 OT-I mice (KO) using EasySep mouse naïve CD8⁺ T cell isolation kit (Stemcell Technologies cat: 19858A). 50,000 naïve OT-I cells were retro-orbitally injected into B6 recipients, which were then retro-orbitally infected with 106 CFU LM-actA-Ova 1 day later. On D80 post infection, splenocytes were harvested from recipients, stained with anti-CD45.1-APC antibody then purified with Mojosort mouse anti-APC nanobeads (Biolegend Cat: 480072). 10⁶ enriched memory OT-I cells were adoptively transferred into MC38-Ova tumor-bearing mice, which were subcutaneously inoculated with 10⁶ MC38-Ova tumor cells 4 days earlier. Tumor areas were measured three times a week.

Confocal microscopy. Cells were mounted using poly-D-lysine, fixed with 2% glutaraldehyde then quenched with 1 mg/mL NaBH4. Cells were then rendered permeable using 0.25% Triton X-100 solution, blocked and stained with polyclonal anti-rabbit TOM20 antibody (abcam ab78547 LOT: GR3199811-2) to label mitochondrial outer membranes, DAPI for nuclear staining. Texas red anti-rabbit IgG (VECTOR TI-1000) was used as a secondary antibody for TOM20 staining. Quantification was performed with Bitplane Imaris software (Oxford Instruments). Outlines were traced manually for each mitochondrion in all images, and Imaris software used to calculate the total mitochondrial volume and surface area for each cell. All microscopy was performed in the Dartmouth Institute for Biomolecular Targeting (BioMT).

ATP detection assay. Naïve CD8⁺ T cells were purified from spleens of CD45.1 OT-I mice (WT) or GZB-cre Zbtb20-fl/fl CD45.1 OT-I mice (KO) using EasySep mouse naïve CD8⁺ T cell isolation kits (StemCell Technologies cat: 19858A). 50,000 naïve OT-I cells were retro-orbitally injected into congenic recipient mice, which were then retro-orbitally infected with 10⁶ CFU LM-actA-Ova 1 day later. On D7 and D28 post infection, splenocytes were harvested from recipients, stained with anti-CD45.1-APC then purified with Mojosort mouse anti-APC nanobeads (Biolegend Cat: 480072). Purified cells (purity greater than 95%) were then analyzed using a luminescence-based ATP detection assay (Cayman Chemical cat: 700410).

Cell preparation for single cell RNAseq. For isolation of CD8⁺ T cells 10 days after infection, single-cell suspensions were generated from four mice per recipient group by macerating spleens through nylon filters. CD8⁺ T cells were enriched from these suspensions using a Stemcell EasySep™ Mouse CD8 T Cell Isolation Kit (#19853). These samples were stained to block Fc receptors then stained with antibodies and live/dead stain (LIVE/DEAD™ Fixable Violet Dead Cell Stain Kit, ThermoFisher #L34955) for 30 minutes on ice shielded from light. The antibodies used for cell surface staining from BioLegend were as follows; PE anti-mouse CD8$ Antibody (YTS156.7.7), APC anti-mouse CD45.1 Antibody (A20) and APC anti-rat CD90/mouse CD90.1 (Thy-1.1) Antibody (OX-7). Samples were subsequently washed twice and ˜1×10⁶ congenically marked OT-I cells were purified using fluorescence activated cell sorting for each group of recipients. The samples purified in this way from each group of recipients were then suspended in 100 μL buffer and labeled with 1 μg per sample of the following Total-seq A antibodies from BioLegend: TotalSeq™-A0198 anti-mouse CD127 (A7R34), TotalSeq™-A0250 anti-mouse/human KLRG1 (2F1/KLRG1), TotalSeq™-A0073 anti-mouse/human CD44 (IM7) and TotalSeq™-A0112 anti-mouse CD62L (MEL-14). Samples were labeled for 30 minutes on ice and subsequently washed with 1 mL PBS twice.

Single-cell RNA Sequencing. Single cell RNAseq library preparation were carried out by the Center for Quantitative Biology Single Cell Genomics Core and the Genomics and Molecular Biology Shared Resource at Dartmouth. Droplet-based 3′-end scRNA-seq was performed using the 10× Genomics Chromium platform, and libraries were prepared using the Single Cell v3 3′ Reagent kit according to the manufacturer's protocol (10× Genomics, CA, USA). Recovery of antibody-DNA tags (ADTs) from single cells (i.e. CITE-seq) was performed by adding 1 ul of ADT additive primer (10 uM, CCTTGGCACCCGAGAATT*C*C) to the cDNA amplification reaction and following the 10× protocol for separation of the ADT and mRNA-derived cDNA fractions. ADT libraries were further amplified using 1 ul SI-PCR primer (10 uM, AATGATACGGCGACCACCGAGATCTACACTCTTTCCCTACACGACGC*T*C) and lui Illumina RPI_X index primer, where X represents a unique index sequence per sample. ADT and mRNA libraries were normalized to 4 uM and pooled at a 1:9 ratio before loading onto a NextSeq 500 instrument. Libraries were sequenced using 75 cycle kits, with 28 bp on read1 and 56 bp for read2.

Data Analysis for Single-cell RNA Sequencing. The Cell Ranger Single-Cell Software Suite (10× Genomics) was used to perform barcode processing and transcript counting after alignment to the mm10 reference genome with default parameters. 7267 cells in the cKO and 10119 cells in the WT were analyzed for 10784 genes. Analysis of the gene-level transcript counts output by Cell Ranger was performed in R (Version 3.5.2) on the merged KO and WT datasets (Manjunath, N., et al., 2001, J. Clin. Invest. 108: 871-878) using the Seurat R package (Version 3.1.4) (Manjunath, N., et al., 2001 (Id.); Frauwirth, K. A., et al., 2002, “The CD28 signaling pathway regulates glucose metabolism”, 2002, Immunity, 16(6):769-77.). All ribosomal genes and genes with counts in fewer than 25 cells were excluded. Cells with mitochondrial DNA content >10% or non-zero counts for fewer than 500 genes or more than 3,000 genes were also removed. The filtered gene expression data was normalized using the SCTransform method and subsequent computations were performed on the matrix of corrected counts. Unsupervised clustering was performed using Seurat's implementation of shared nearest neighbor (SNN) modularity optimization with the resolution parameter set to 0.2 (Hudson, W. H., et al., 2019, Immunity 51: 1043-1058.e4). For data visualization, single cell gene expression data were projected onto a reduced dimensional space as computed by the Uniform Manifold Approximation and Projection (UMAP) method (Böttcher, J. P., et al., 2015, Nat Commun 6: 8306) for the first 30 principal components of the expression data. The Variance-adjusted Mahalanobis (VAM) method (Frost, H. R. “Variance-adjusted Mahalanobis (VAM): a fast and accurate method for cell-specific gene set scoring”, 2020, Nucleic Acids Res. 48(16):e94.) was used to compute cell-specific scores for pathways from Molecular Signature Database collections (MSigDB; Version 7.0) that were filtered to remove pathways with fewer than 5 members or more than 200 members. We identified differentially expressed genes and pathways between KO and WT cells using Wilcoxon rank sum tests applied to either the normalized counts for each gene or the VAM scores for each pathway with p-values adjusted using the Bonferroni method.

Reagents: EasySep Mouse naïve CD8 T cell isolation kits (Stemcell Technologies cat: 19858A); Mojosort mouse anti-APC nanobeads (Biolegend Cat: 480072); ATP detection assay kit-luminescence (Cayman Chemical cat: 700410); DAPI (Thermo Fisher cat: D1306); Seahorse XF Cell Mito Stress Test Kit (Seahorse Agilent cat: 103015-100); 2-DG (Cayman Chemical cat: 14325); SIINFEKL peptide (New England peptide Lot: V1355-37/40); recombinant human IL-2 (TECIN cat: Ro23-6019); recombinant murine IL-15 (PeproTech cat: 210-15); poly-D-lysine (Millipore Sigma cat: P6407); Glutaraldehyde (Electron Microscopy Science cat: 16000); NaBH4 (Alfa Aesar stock #: 35788); Triton X-100 (PerkinElmer cat: N9300260).

Antibodies: violet fluorescent reactive dye (life technologies ref: L34955); CD45.1-BV421 (Biolegend cat: 110732); Blimpl-BV421 (BD Bioscience cat: 564270); CD8-BV510 (Biolegend cat: 100752); CD45.1-BV510 (Biolegend cat: 110741); CD45.1-APC (Biolegend cat: 110714); CD62L-BV510 (Biolegend cat: 104441); CD127-BV510 (Biolegend cat: 135033); CD8-BV650 (Biolegend cat: 100742); MitoTracker-Green FM (Invitrogen ref: M7514); CD62L-FITC (eBioscience cat: 11-0621-85); Thy1.1-A488 (Biolegend cat: 202506); Thy1.1-APC (Biolegend cat: 202526); TCF1-A488 (cell signaling ref: 02/2018); TNFa-FITC (Biolegend cat: 506304); MITOsox Red mitochondrial superoxide indicator (Invitrogen ref: M36008); CD45.2-PE (Biolegend cat: 109808); CD62L-PE (Biolegend cat: 104408); CD127-PE (Biolegend cat: 135010); EOMES-PE (invitrogen ref: 12-4875-82); IL2-PE (Biolegend cat: 503808); Thy1.1-PE (Biolegend cat: 202524); TNFa-PE (Biolegend cat: 506306); CD8-PerCPcy5.5 (Biolegend cat: 100734); CD44-PerCPcy5.5 (Invitrogen ref: 45-0441-82); Bcl6-PerCPcy5.5 (BD Pharmingen cat: 562198); IFNy-PerCPcy5.5 (Biolegend cat: 505822); Thy1.1-PEcy7 (Biolegend cat: 202518); KLRG1-PEcy7 (Biolegend cat: 138416); CD27-PEcy7 (Biolegend cat: 124216); Tbet-PEcy7 (Invitrogen ref: 25-5825-82); GZB-PEcy7 (eBioscience ref: 25-8898-82); CD25-APC (Biolegend cat: 102008); CD44-APC (Biolegend cat: 103012); CXCR3-APC (Biolegend cat: 126512); IFNy-APC (Biolegend cat: 505810); Thy1.1-APC (Invitrogen ref: 17-0900-82); p79-APC tetramer (NIH tetramer facility) Bcl2-A647 (Biolegend cat: 633510); Bcl6-A647 (BD Pharmingen cat: 561525); CD8-APCef780 (eBioscience; REF 47-0081-82); near-IR fluorescent reactive dye (Invitrogen ref: L10119); poly clonal anti-rabbit TOM20 (Abcam ab78547 LOT: GR3199811-2); Texas red anti-rabbit IgG (VECTOR TI-1000): TotalSeq™-A0198 CD127 (BioLegend, cat: 135045); TotalSeq™-A0073 CD44 (BioLegend, cat: 103045); TotalSeq™-A0112 CD62L (Biolegend cat: 104451).

The following examples are provided for illustrative purposes only and are non-limiting.

### Example 1: Zbtb20 Deficiency Negatively Regulates Mitochondrial Metabolism in CD8⁺ T Cells

Zbtb20 belongs to the evolutionarily conserved BTB-ZF transcription factor family. The cDNA and amino acid sequences for human Zbtb20 are provided in SEQ ID NO: 1 and SEQ ID NO: 2, respectively, and the cDNA and amino acid sequences for mouse Zbtb20 are provided in SEQ ID NO: 3 and SEQ ID NO: 4, respectively. There are more than 49 BTB-ZF genes in mammals, characterized by one or more C-terminal C2H2 zinc finger DNA binding domains in combination with an N-terminal BTB domain that mediates protein-protein interactions (Siggs and Beutler (2012) Cell Cycle, 11(18):3358-69. doi:10.4161/cc.21277; Beaulieu, et al. (2011) J. Immunol. 187(6):2841-7). Transcriptional regulation, commonly repression, is achieved by sequence-specific binding by the ZF domain to regulatory regions adjacent to target genes, followed by the recruitment of co-factors by the BTB domain which can mediate chromatin remodeling or transcriptional silencing. BTB-ZF proteins, including BCL-6, PLZF, BAZF and Zbtb20 play critical roles in a wide range of biological process including developmental events, cell cycle progression in normal and oncogenic tissues and maintenance of the stem cell pool. More importantly, many BTB-ZF proteins, like Bcl-6 and BAZF, are also key factors in the development and function of lymphocytes and myeloid cells. Zbtb20 was first identified in human dendritic cells and given the name “dendritic cell-derived BTB/POZ zinc finger (DPZF) (Zhang et al. (2001) Biochem. Biophys. Res. Commun., 282(4):1067-73). A homolog of Bcl-6, Zbtb20 is widely expressed in hematopoietic tissues and neuronal tissues. It has been shown that Zbtb20 promotes antibody-secreting B cell longevity and differentiation and is indispensable for maintaining the long-lived plasma cell response (Chevrier et al. (2014) J. Exp. Med., 211(5):827-40). In addition, Zbtb20 induces cell survival factors including Bcl-2, Bcl-w, Bcl-x and blocks cell cycle progression in a plasma cell line. Global Zbtb20 deficiency causes neonatal death of mice due to growth retardation and metabolic dysfunction (Sutherland et al., (2009) Mol. Cell. Biol., 29(10):2804-15). Transcriptional profiling of liver tissue from Zbtb20 KO pups revealed dysregulation of a number of genes related to metabolism and mitochondria function, including AKT, PGC1α, PDK4, CPT, PI3K, and fatty acid synthase.

OT-I mice were used for the mouse studies described herein. As used herein, “OT-I mice” refers to mice containing transgenic inserts for mouse Tcra-V2 and Tcrb-V5 genes encoding a transgenic T cell receptor which recognizes ovalbumin peptide residues 257-264 (OVA₂₅₇₋₂₆₄) in the context of H2K^{b} (CD8⁺ co-receptor interaction with MHC class 1). This results in MHC class I-restricted, ovalbumin-specific, CD8⁺ T cells (referred to herein as “OT-I cells”). That is, the CD8+ T cells of this mouse primarily recognize OVA₂₅₇₋₂₆₄ when presented by the MHC I molecule. Immune response dynamics can be studied by in vivo adoptive transfer or in vitro co-culture with cells transgenic for ovalbumin or by direct administration of ovalbumin. OT-I mice are suitable for the study of CD8⁺ T cell response to antigen, positive selection, and for any research requiring CD8⁺ T cells of defined specificity. Like most TCR transgenic mice, OT-I mice are somewhat immunodeficient. Within this disclosure, OT-I mice and OT-I cells which have not been further genetically modified are referred to as wild-type, e.g., “WT OT-I” mice and cells, respectively.

As there was the potential for Zbtb20 deletion to affect naïve CD8⁺ T cell function, a GZB-cre ZBTB20-f/f conditional knockout OT-I transgenic mouse model was used, where Zbtb20 is deleted in CD8⁺ T cells only after T cell activation. The Zbtb20 conditional knockout OT-I mice and OT-I cells are referred to herein as “KO OT-I” mice and cells, respectively.

The effects of Zbtb20 deletion on metabolism in effector and memory CD8⁺ T cells were investigated. Total splenocytes were harvested from either KO or WT OT-I mice, then seeded at 2×10\hat{ }⁶ cells/mL with 10 ug/mL SIINFEKL peptide for 48 h without exogenous IL-2. Activated cells were further cultured at 0.5×10\hat{ }⁶ cells/mL with 100 U/mL recombinant human IL-2 or at 1×10\hat{ }⁶ cells/mL with 50 ug/mL recombinant mouse IL-15 for 7 days. Cultures were split every 2-3 days.

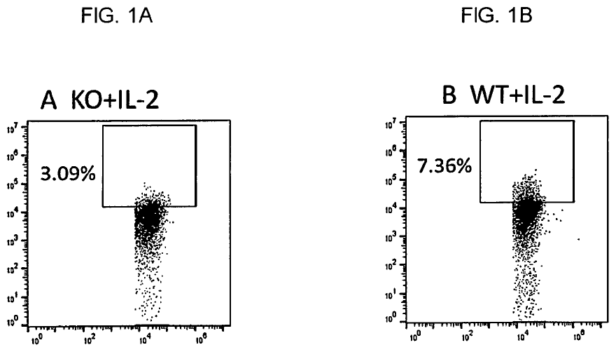

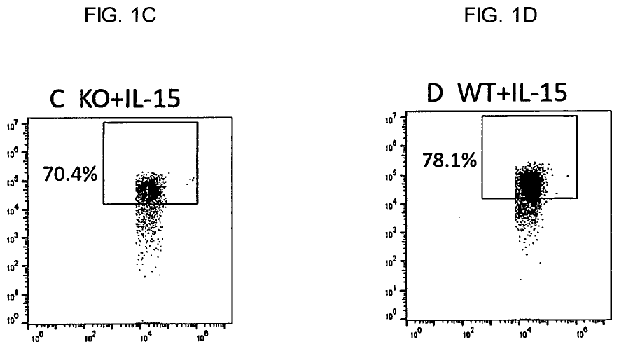

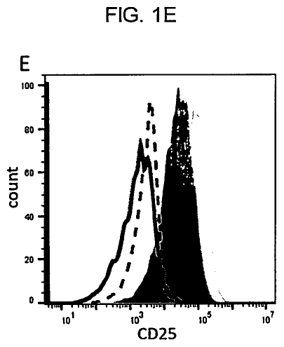

Consistent with previous reports, culture with IL-2 induced T_{eff}-like cells, which are characterized by high expression of CD25 and low expression of CD62L, and culture with IL-15 induced T_{cm}-like cells, which express low levels of CD25 and high levels of CD62L (FIG. 1A-FIG. 1E).

WT and KO CD8⁺ OT-I cells were then subjected to metabolic analysis to test mitochondrial respiration and glycolytic metabolism using the Seahorse XFe96 Bioanalyzer (Agilent). In this experiment, 150,000 cells were seeded per well for the IL-2 or IL-15 in vitro differentiated CD8⁺ T cells described above. The Seahorse XF Cell Mito Stress Test Kit and Seahorse XF Glycolytic Rate Assay Kit were used according to the manufacturer's protocols.

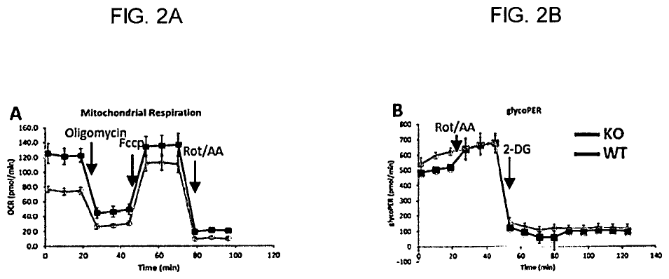

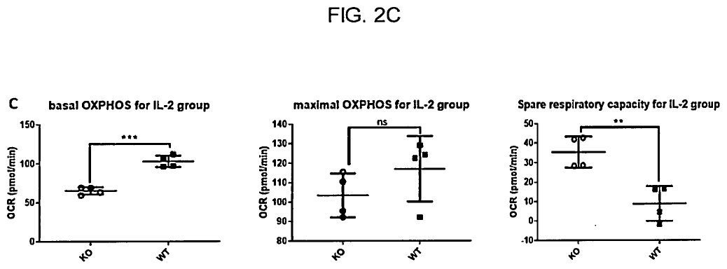

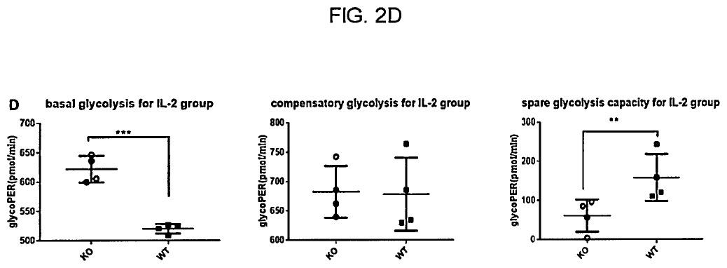

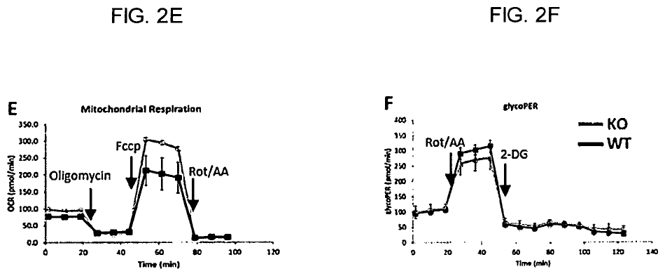

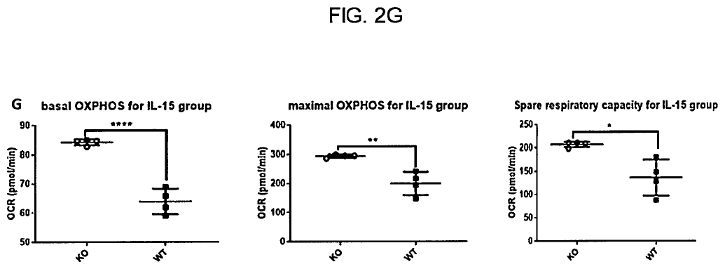

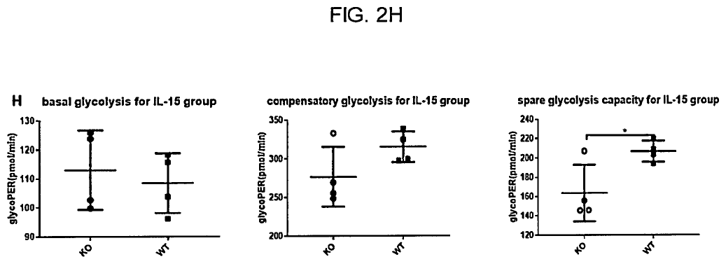

Results for cells cultured with IL-2 (i.e., T_{eff} cells) were as follows: KO T_{eff} cells had significantly lower basal mitochondrial respiration, indicated by lower basal oxygen consumption rate (OCR), compared with WT T_{eff} cells but maximal respiration was not different between WT and KO T_{eff} cells (FIG. 2A, FIG. 2C). This resulted in a higher spare respiratory capacity in KO T_{eff} cells compared to WT T_{eff} cells. The glycolytic capacity (glycoPER) of KO and WT T_{eff} cells was also interrogated, as effector CD8⁺ T cell are known to heavily depend on glycolysis for production of ATP and effector functions. KO T_{eff} cells displayed higher basal glycolysis compared with WT T_{eff} cells, but maximal glycolytic capacity (compensatory glycolysis) was not different between the groups. This resulted in little spare glycolytic capacity (SGC) in KO T_{eff} cells in contrast to WT T_{eff} cells which possessed significantly higher SGC (FIG. 2B, FIG. 2D).

Taken together, the data suggested that in vitro generated KO T_{eff} cells had the same maximal capacity for performing glycolysis as well as mitochondrial respiration as WT T_{eff} cells. However, under basal conditions KO T_{eff} cells displayed higher glycolytic activity and lower mitochondrial respiration.

Results for cells cultured with IL-15 (i.e., T_{cm} cells) were as follows: WT T_{cm} cells had higher spare respiratory capacity (SRC) compared with T_{eff} cells (FIG. 2A, FIG. 2E). KO T_{cm} cells displayed higher basal mitochondrial respiration, higher maximal respiration, as well as higher SRC when compared with WT T_{cm} cells (FIG. 2E, FIG. 2G). KO T_{cm} cells displayed similar basal glycolysis and compensatory glycolysis but significantly lower SGC compared with WT T_{cm} cells (FIG. 2F, FIG. 2H).

Collectively, these data show that Zbtb20 deletion increased spare mitochondrial respiratory capacity in both T_{eff} cells and T_{cm} cells. In contrast, deletion of Zbtb20 decreased spare glycolytic capacity in both T_{eff} cells and T_{cm} cells. Interestingly, Zbtb20 deletion had opposite effects on basal mitochondrial respiration in T_{eff} cells and T_{cm} cells, but only altered basal glycolysis in T_{eff} cells. This demonstrated that Zbtb20 is an important regulator of both glycolysis and mitochondrial respiration.

### Example 2: Zbtb20-Deficient Memory CD8⁺ T Cells have Increased Mitochondrial Mass

To determine whether enhanced mitochondrial metabolism observed in KO T_{eff} cells or T_{cm} cells was accompanied by increased mitochondrial content, in vitro generated T_{eff} cells or T_{c}m cells, differentiated in IL-2 or IL-15 as above, respectively, were fixed then stained with DAPI and TOM20 antibody to visualize the mitochondrial outer membrane. Examination by confocal microscopy was used to quantify mitochondrial surface area and volume. Specifically, cells were mounted using poly-D-lysine, fixed with 2% Glutaraldehyde, then quenched with 1 mg/mL NaBH₄. Cells were then permeabilized using 0.25% Triton X-100 solution, blocked and stained with poly clonal anti-rabbit TOM20 for mitochondria outer membrane and DAPI for nucleus. Texas red anti-rabbit IgG was used as a secondary antibody for TOM20. Quantification was performed with Imaris 10.0 software.

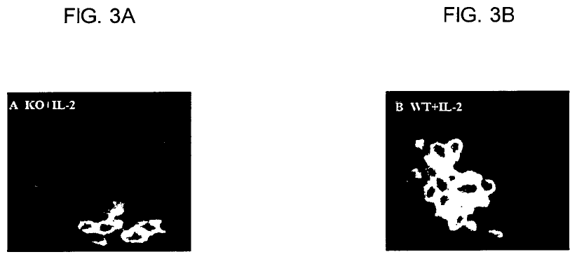

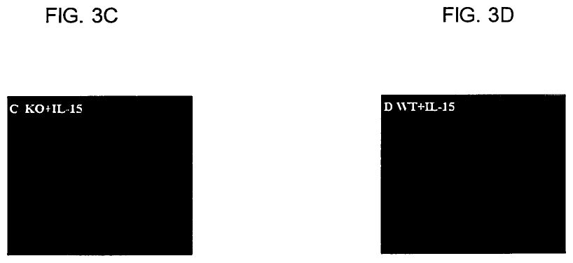

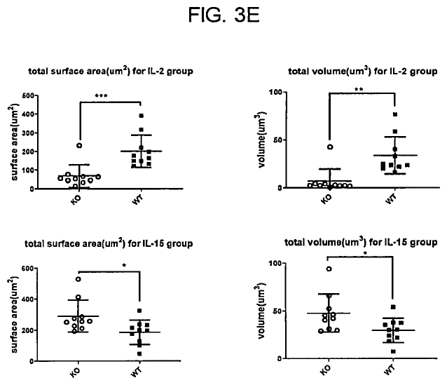

This revealed that KO T_{eff} cells had less mitochondrial surface area and volume than WT T_{eff} cells, whereas KO T_{cm} cells had larger mitochondrial surface area and volume than WT Tcm cells (FIG. 3A-FIG. 3E). Therefore, both mitochondrial size and oxidative phosphorylation potential (SRC) were increased in Zbtb20-deficient memory CD8⁺ T cells.

### Example 3: Enhanced Glycolysis and Mitochondrial Respiration in Zbtb20-Deficient CD8⁺ T Cell Responses Ex Vivo

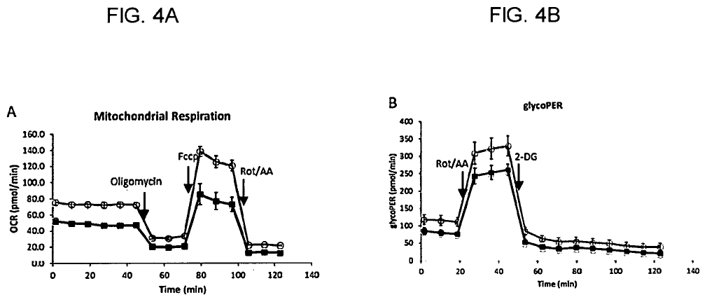

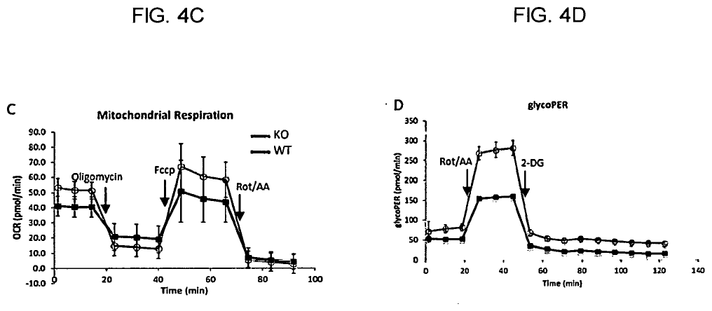

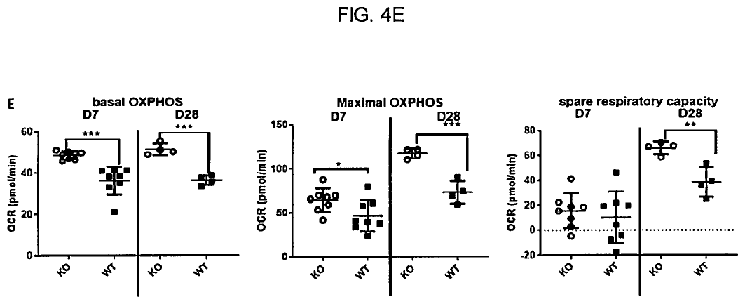

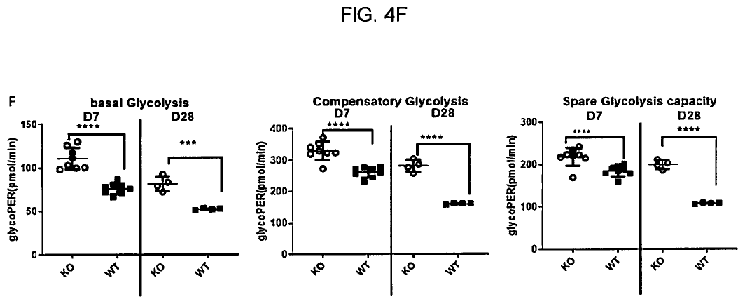

Naïve CD8⁺ T cells (defined as CD62L⁺/CD44⁻) from either KO CD45.1 OT-I donor mice or WT CD45.1 OT-I donor mice were purified, then adoptively transferred into recipient CD45.2 mice subsequently intravenously infected with an OVA-expressing actA⁻ strain of Listeria monocytogenes (LM-actA-OVA). Splenocytes were harvested from CD45.2 recipient mice at day 7 post-infection (to obtain effector T cells) or day 28 post-infection (to obtain memory T cells) and CD45.1 positive OT-I cells were magnetically selected. Purified cells were then assayed for mitochondrial respiratory and glycolytic rates. Strikingly, both effector and memory CD8⁺ T cells had higher basal and maximal mitochondrial respiration compared with WT (FIG. 4A and FIG. 4C). Zbtb20 KO memory, but not effector, T cells also had higher spare respiratory capacity compared with WT (FIG. 4A, FIG. 4C, and FIG. 4E). In addition, both effector and memory Zbtb20 KO CD8⁺ T cells exhibited higher basal and maximal glycolysis as well as spare glycolytic capacity (FIG. 4B, FIG. 4D, and FIG. 4F). These data indicated that Zbtb20 KO effector and memory CD8⁺ T cells directly taken from infected animals were in a more energetic state, caused by upregulated mitochondrial metabolism and glycolysis.

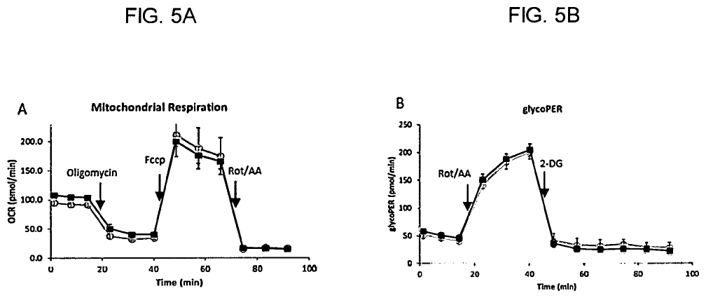

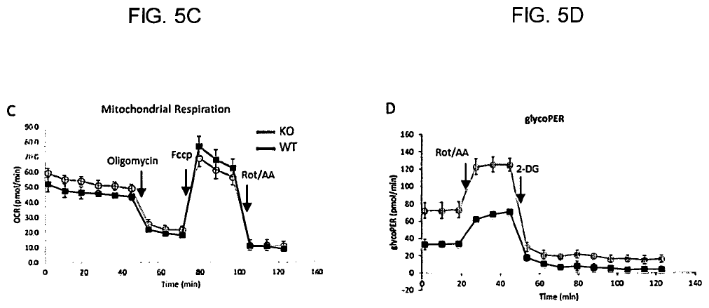

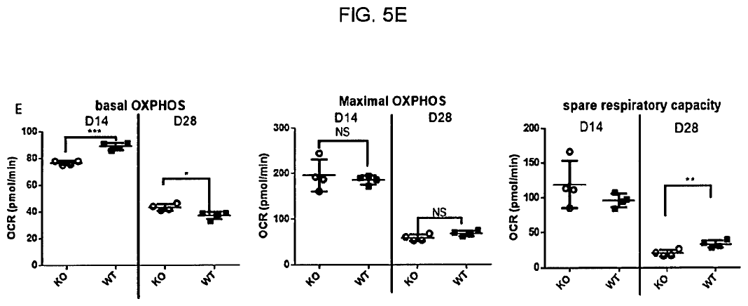

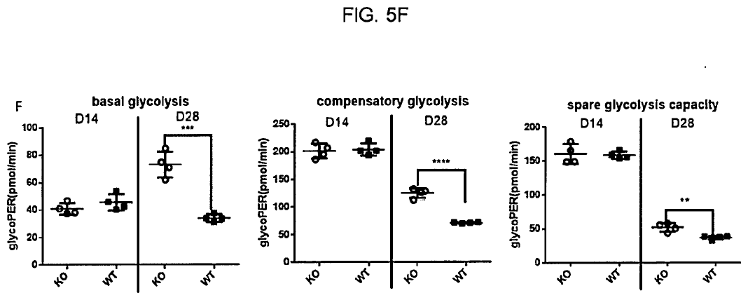

Consistent with the LM model, Zbtb20 KO memory CD8⁺ T cell in the murine gammaherpesvirus (MHV-68) infection model also had superior glycolytic capacity as well as basal OXPHOS (FIG. 5A-FIG. 5F).

### Example 4: Increased ATP Content and Higher Mitochondria Mass Ex Vivo in the Absence of Zbtb20

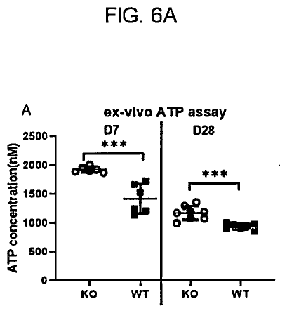

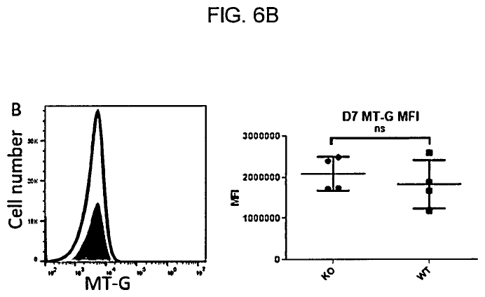

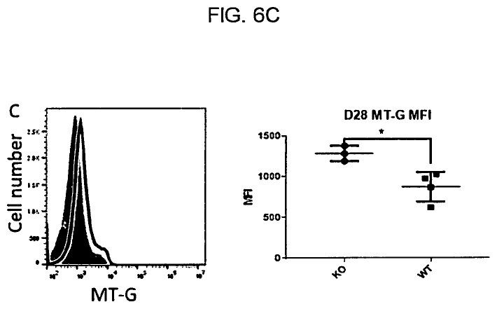

The ATP content in WT and Zbtb20-deficient CD8⁺ T cells was measured. Splenocytes from recipient mice were harvested on 7 or 28 days post infection and CD45.1 positive OT-I cells were magnetically purified. Purified WT or Zbtb20 KO OT-I cells were then used in a luminescence-based ATP detection assay. The results indicated that ex vivo enriched effector and memory Zbtb20 KO CD8⁺ T cells consistently had higher ATP content than WT cells (FIG. 6A).

Mitochondrial mass was also measured ex vivo by staining with the mitochondrial dye Mitotracker Green. The results indicated that Zbtb20 KO OT-I cells had the same mitochondrial content at day 7 (FIG. 6B), but higher mitochondrial content at day d28 post-infection (FIG. 6C).

### Example 5: Zbtb20 is Induced in Activated CD8⁺ T Cells

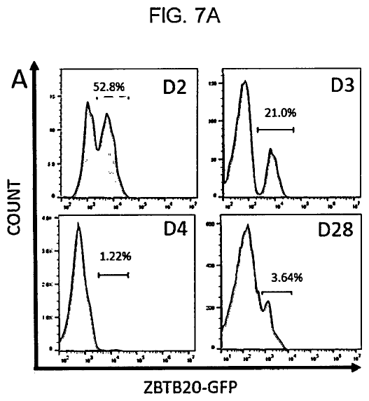

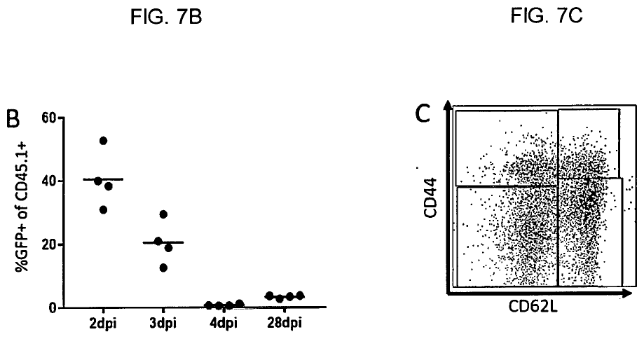

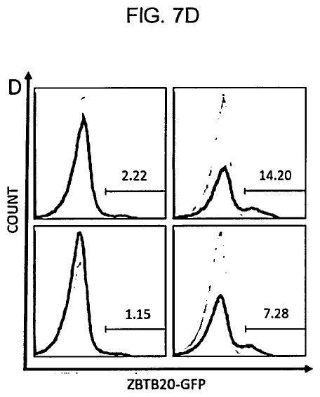

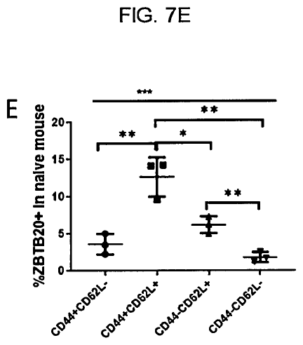

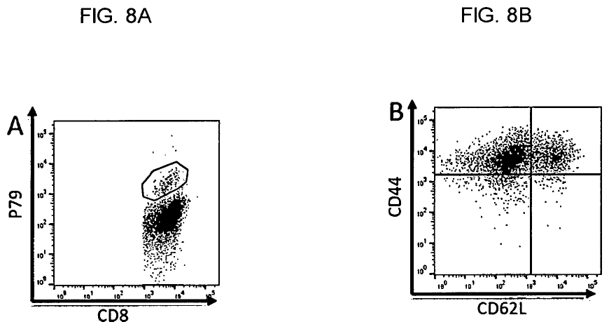

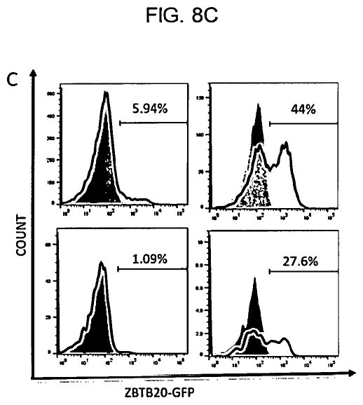

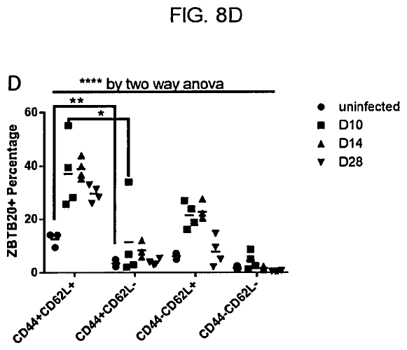

In order to dissect the expression pattern of Zbtb20 in CD8⁺ T cells, a Zbtb20 reporter mouse strain that has GFP expressed from the Zbtb20 promoter was used. Naïve (CD62L⁺CD44⁻) OT-I cells from ZBTB20-GFP CD45.1 OT-I reporter donor spleens were adoptively transferred to CD45.2 recipient mice. Recipient mice were then intravenously infected with 10⁶ CFU LM⁻actA⁻OVA the following day. Splenocytes were harvested from recipient mice on day 2, 3, 4 and 28 post-infection for analysis. Zbtb20 was expressed in approximately half of the CD8⁺ T cell population on D2 post infection then the proportion of positive cells decreased at D3 and was very low by D4 post infection (FIG. 7A-FIG. 7B). However, by D28 the Zbtb20 reporter was again detectable in a small proportion of cells. To identify populations expressing Zbtb20 in vivo, splenocytes from naïve ZBTB20-GFP mice were harvested. It was observed that the phenotype with the highest proportion of Zbtb20 expressing cells (^{˜}12%) was naturally occurring T_{cm} (defined as CD44⁺CD62L⁺). Naïve CD8⁺ T cells (defined as CD44⁻CD62L⁺) also contained ^{˜}6% Zbtb20 expressing cells. However, CD44⁺CD62L⁻ and CD44⁻CD62L⁻ CD8⁺ T cells contained low proportions of cells expressing Zbtb20 (FIG. 7C-E). The expression pattern of Zbtb20 in the MHV-68 infection model was also investigated. ZBTB20-GFP reporter mice were intra-nasally infected with MHV-68. Splenocytes were harvested before infection and on day 10, day 14 and day 28 post infection then GFP expression in the polyclonal CD8⁺ T cell population staining with a tetramer folded with the dominant ORF61 (P79) epitope was measured. The results indicated the highest proportion of Zbtb20 expressing cells in the CD44⁺CD62L⁺ central memory population, followed by CD44⁻CD62L⁺ naïve CD8⁺ T cells (FIG. 8A-D).

### Example 6: Zbtb20 Deletion Enhances Cytokine Production and Favors Memory Precursor Differentiation

Given Zbtb20 expression at the early stages of effector differentiation and in a subset of central memory phenotype cells, how Zbtb20 deficiency affected effector and memory differentiation in vivo was tested.

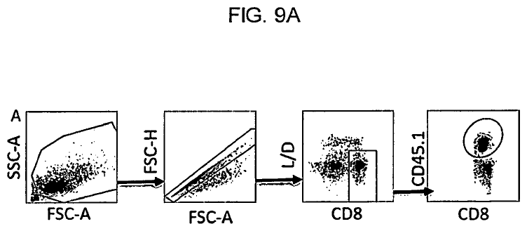

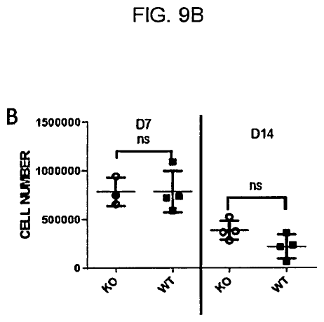

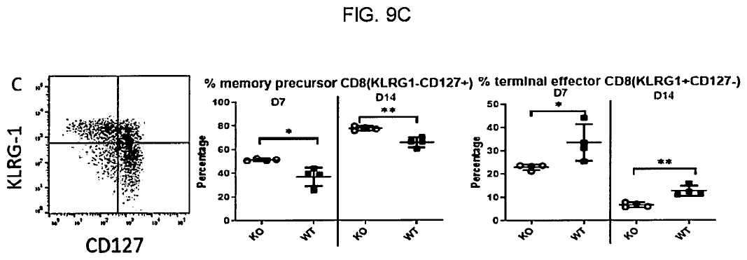

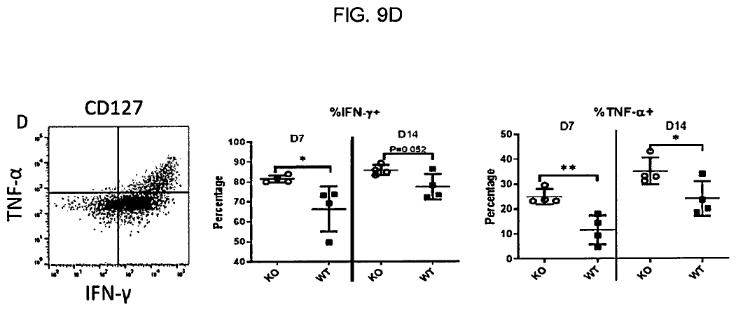

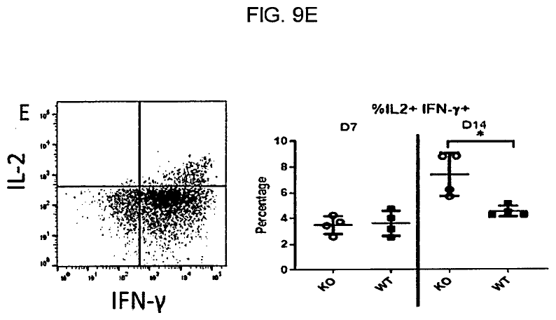

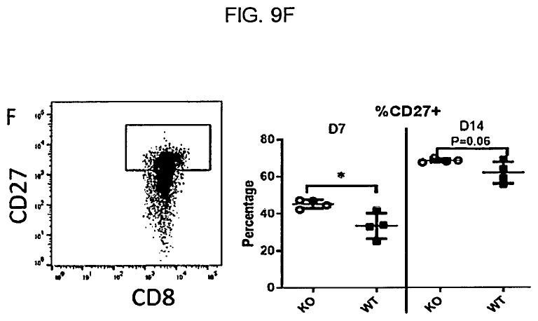

To determine how Zbtb20 deletion affected CD8⁺ T cell clonal expansion, accumulation, function and differentiation, naïve OT-I cells from either GZB-cre ZBTB20-f/f CD45.1 OT-I (KO) or CD45.1 OT-I (WT) donor mice were purified and either naïve KO OT-I or WT OT-I cells were adoptively transferred into recipient CD45.2 mice which were then intravenously infected with LM-actA-OVA. Splenocytes from recipient mice were harvested for analysis on various days post infection. The number of transferred OT-I cells recovered from the spleens of recipient were the same at both D7, which measures the peak CD8⁺ T cell response against LM, and D14, which is during the contraction phase (FIG. 9A-9B). Examining the phenotype of responding OT-I T cells revealed that on both D7 and D14 post infection, Zbtb20 KO OT-I cells were more skewed towards memory precursors (defined as KLRG-1⁻/CD127⁺) than terminally differentiated effectors (defined as KLRG-1⁺/CD127⁻) (FIG. 9C). In addition, cytokine production profiles revealed that a higher proportion of Zbtb20 KO OT-I cells could produce IFN-γ or TNF-α as well as both IL-2 and IFN-γ simultaneously (FIG. 9D-FIG. 9E). Production of IL-2 is a characteristic of memory cells, consistent with memory precursor skewing. A larger proportion of KO cells expressing high levels of CD27, which is preferentially expressed on central memory CD8⁺ T cells, was also detected (FIG. 9F). Additionally, a larger proportion of Zbtb20 KO effector CD8⁺ T cell expressed high levels of CXCR3 during the contraction phase (FIG. 9G), an important chemokine receptor that drives effector CD8⁺ T cell to sites of inflammation. Taken together, these data suggested that Zbtb20 KO effector CD8⁺ T cell had increased memory potential and enhancements in cytokine production.

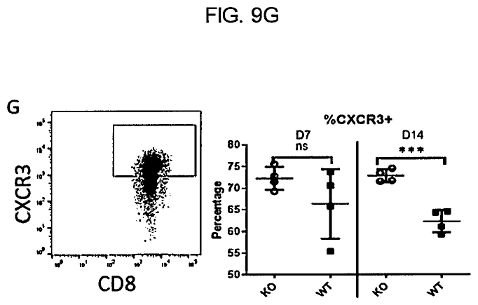

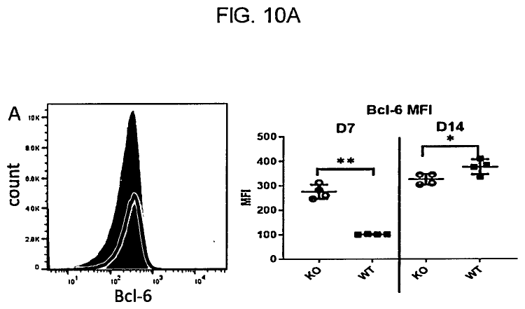

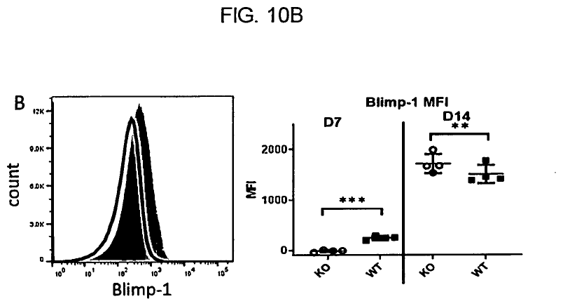

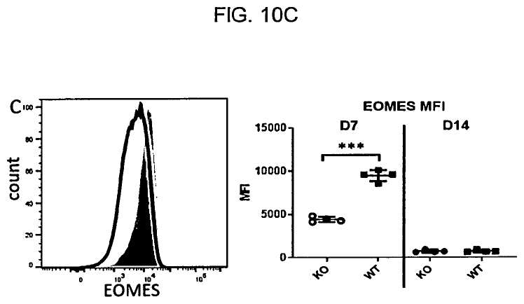

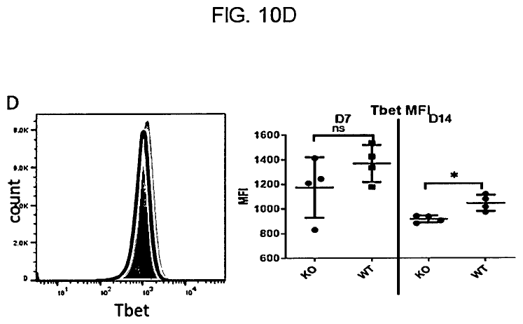

A network of transcription factors tightly orchestrates differentiation of effector and memory CD8⁺ T cells. These regulate the expression of crucial cytokine receptors, pro-apoptotic and anti-apoptotic factors, cellular metabolism and other critical functions. Interrogation of transcription factor expression revealed that Zbtb20 KO effector CD8⁺ T cells expressed higher levels of Bcl-6 and lower levels of Blimp-1 on D7, whereas on D14 KO effector CD8⁺ T cell expressed lower Bcl-6 and higher Blimp-1 compared with WT (FIG. 10A-FIG. 10B). In addition, Zbtb20 KO effector CD8⁺ T cells had lower expression of Eomes, a transcription factor which favors memory CD8⁺ T cell differentiation, on D7 but not D14 (FIG. 10C). We also observed that T-bet, a transcription factor related to effector CD8⁺ T cell differentiation, was expressed at a lower level in Zbtb20 KO effector CD8⁺ T cells on D14 but not D7 (FIG. 10D). Collectively, these data suggested that Zbtb20 affects expression of several transcription factors important for effector and memory CD8⁺ T cell differentiation.

### Example 7: Zbtb20 Deletion Affects Memory CD8⁺ T Cell Phenotype and Cytokine Production

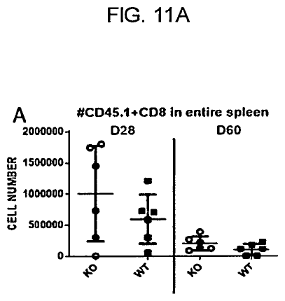

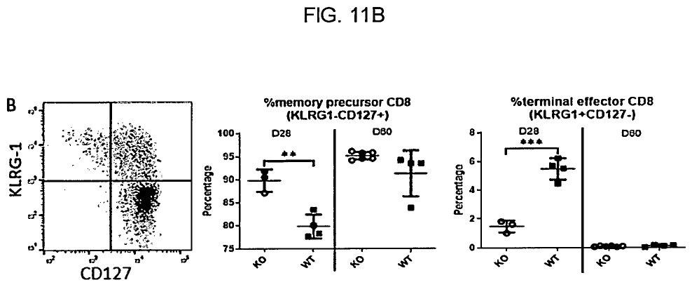

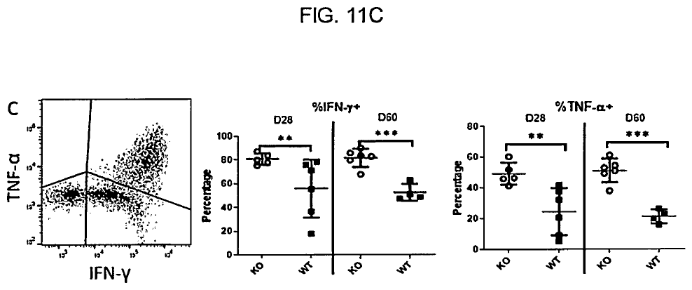

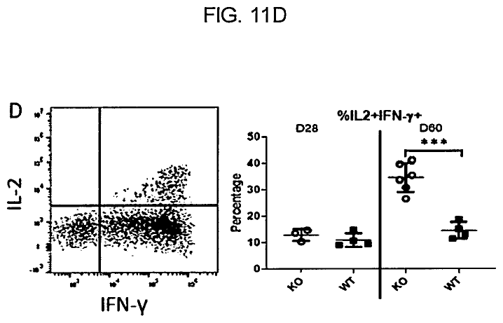

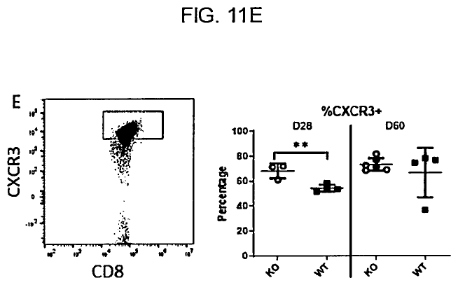

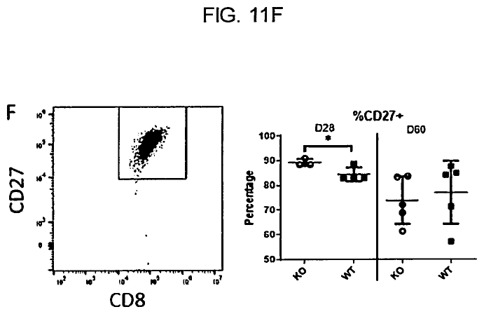

Using the OT-I transfer LM-ActA-ova infection model described above, Zbtb20 KO and WT OT-I cells were tracked until later times post infection, which allowed investigation of the role of Zbtb20 in CD8⁺ T cell memory. On D28 and D60, the number of Zbtb20 KO memory OT-I cells were found to be the same as WT OT-I cells (FIG. 11A). Consistent with earlier times after infection, Zbtb20 KO OT-I cells were more skewed towards memory precursors than effector cells on D28 (FIG. 11B). In addition, more Zbtb20 KO memory OT-I cells could produce IFN-γ or TNF-α (FIG. 11C) as well as both IL-2 and IFN-γ simultaneously (FIG. 11D). Moreover, more Zbtb20 KO memory OT-I cells expressed high levels of CXCR3 and CD27 on D28 (FIG. 11E-FIG. 11F). Therefore, the phenotype indicating skewing toward memory CD8⁺ T cells was consistent with earlier times after infection.

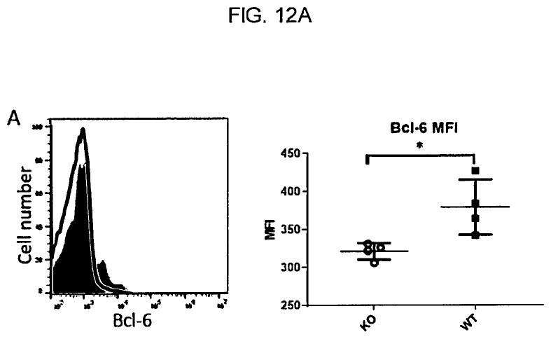

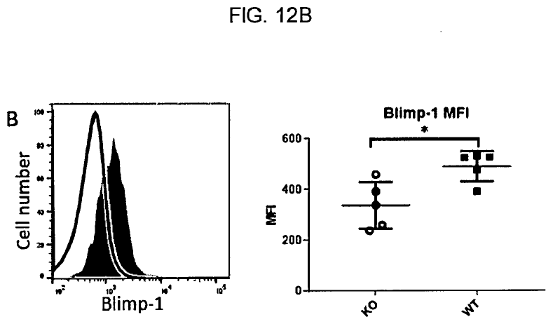

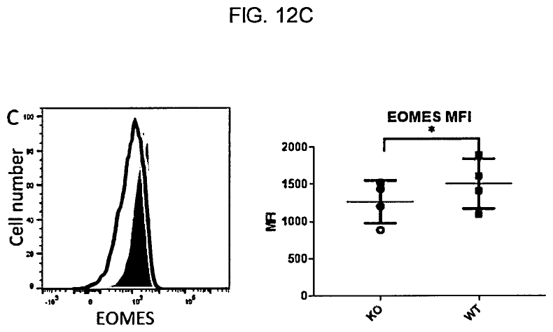

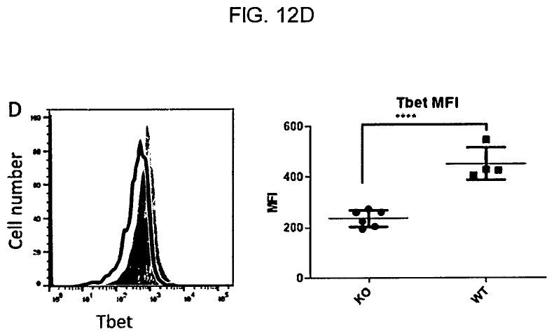

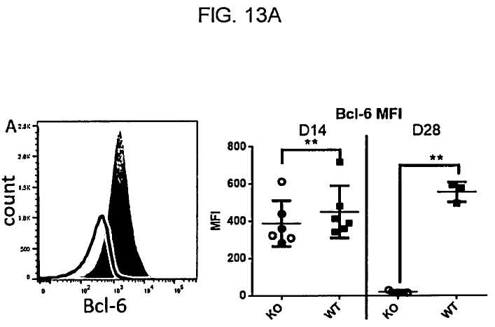

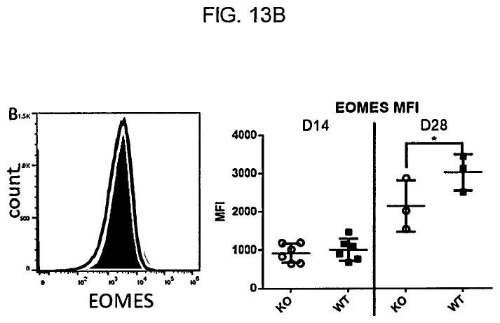

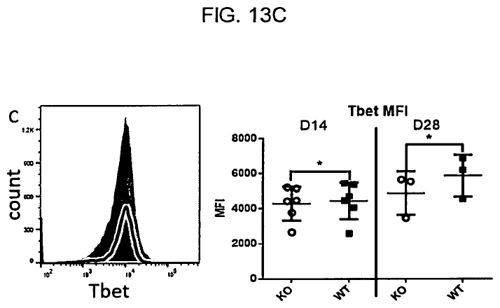

Investigation of transcription factor expression in Zbtb20 KO and WT memory CD8⁺ T cells on D28 revealed that Zbtb20 KO memory cells expressed lower levels of Bcl-6, Blimp-1, EOMES and T-bet (FIG. 12A-FIG. 12D). This indicates disruption of key transcription factors associated with memory is observed both during the effector and memory stages of the CD8⁺ T cell response. Consistent with data from the LM infection model, Zbtb20 KO effector and memory cells expressed lower levels of Bcl-6, EOMES and T-bet following MHV-68 infection (FIG. 13A-FIG. 13C).

### Example 8: Zbtb20 KO Memory CD8⁺ T Cells Mount a More Efficient Secondary Response

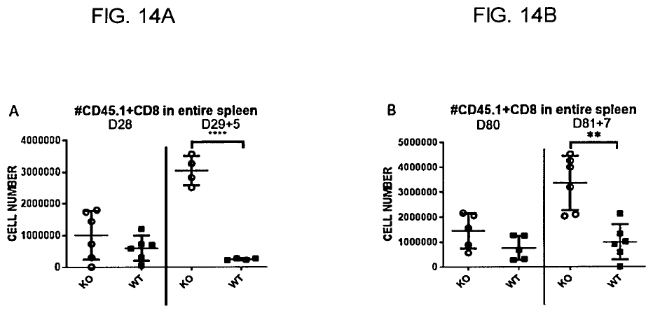

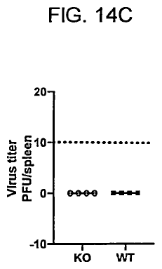

As the previous data indicated the absence of Zbtb20 enhanced differentiation toward memory CD8⁺ T cells, the capacity of Zbtb20 KO and WT memory CD8⁺ T cells to accumulate following secondary antigenic challenge was tested. Within the same experimental design, groups of recipient mice were intravenously re-challenged on D29 or D81 post infection with MHV-68-OVA. FIG. 14A-FIG. 14B shows numbers of OT-I cells both before and five days following challenge. The secondary infection was insufficient to induce a detectable secondary response from WT memory cells, however Zbtb20 KO memory CD8⁺ T cells expanded robustly upon re-challenge at both timepoints. Both Zbtb20 KO and WT OT-I cells cleared the MHV-68-OVA completely within 5 days after re-challenge (FIG. 14C).

### Example 9: Memory CD8⁺ T Cells Lacking Zbtb20 Control MC38 Tumor Growth More Efficiently Compared to WT Memory CD8+ T Cells

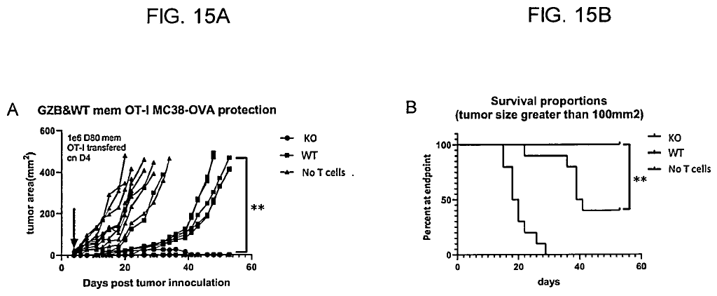

Memory WT or Zbtb20 KO OT-I cells were purified from donor mice infected with LM-OVA 80 days prior to adoptive transfer into B6 recipient mice which had been injected with MC38-OVA tumor cells four days prior to receiving the transferred cells. Tumors grew rapidly in all tumor-bearing mice that received no T cells (FIG. 15A and FIG. 15B). Tumor growth was slower in the majority of mice which received WT memory OT-I cells, but the majority of these mice eventually succumbed. In contrast, Zbtb20-deficient OT-I cells prevented tumor growth in all recipients of these cells. Thus, memory CD8⁺T cells lacking Zbtb20 were significantly more protective against tumor growth when compared with WT memory cells.

### Example 10: Adoptive Cell Therapy with Zbtb20 Suppression in a Human Subject

Immune cells are obtained from a human subject having at least one cancer. The immune cells are preferably T cells obtained from the subject, e.g., from the subject's peripheral blood mononuclear cells obtained via phlebotomy or apheresis. The T cells can be further selected for the presence or absence of one or more markers, such as CD8⁺/CD45RA⁺ (e.g., naïve CD8⁺ T cells) or CD8⁺/CD45RO⁺ (e.g., antigen-experienced CD8⁺ T cells). The subject optionally undergoes a lymphodepletion procedure, which can include low-dose total body irradiation, chemotherapy such as cyclophosphamide and/or fludarabine, and/or hematopoietic stem cell transplantation, after the T cells are obtained from the subject and prior to reinfusion of the modified T cells into the subject. The T cells are modified ex vivo to suppress endogenous Zbtb20 expression and/or activity using one or more of several approaches described below. The T cells are optionally cultured and expanded ex vivo prior to, simultaneously with, and/or after being modified. The T cells may also be cryopreserved prior to and/or after being modified and subsequently thawed prior to being administered to the subject.

The approaches for suppressing endogenous Zbtb20 expression and/or activity include (1) use of a dominant negative Zbtb20 capable of suppressing endogenous Zbtb20 activity in the modified cells; (2) use of at least one shRNA capable of suppressing endogenous Zbtb20 expression in the modified cells; and (3) use of at least one sgRNA capable of suppressing endogenous Zbtb20 expression in the modified cells.

For approach (1), the dominant negative Zbtb20 comprises one or more Zbtb20 C-terminal zinc-finger domains and lacks at least a portion of a Zbtb20 N-terminal region comprising a Zbtb20 BTB domain. For example, the dominant negative Zbtb20 comprises an amino acid sequence that is at least 75% identical, at least 80% identical, at least 85% identical, at least 90% identical, at least 95% identical, at least 98% identical, or at least 99% identical to SEQ ID NO: 40. The dominant negative Zbtb20 is delivered to the T cells using any technique for delivering proteins to mammalian cells, such as expression of the dominant negative Zbtb20 fused with a cell-penetrating peptide sequence and/or use of cationic lipids.

Alternatively, the T cells are genetically engineered to express the dominant negative Zbtb20. Any genetic engineering technique is used. For example, the genetic engineering approach is selected from a CRISPR/Cas-based genetic engineering method, a TALEN-based genetic engineering method, a ZF-nuclease genetic engineering method, and a transposon-based genetic engineering method.

Alternatively, a nucleic acid encoding the dominant negative Zbtbt20 is delivered to the T cells using any technique for delivering nucleic acids to mammalian cells, such as use of cationic lipids, viral particles, electroporation, and microinjection. The nucleic acid is any nucleic acid suitable for expressing a protein in a mammalian cell. For example, the nucleic acid is selected from an in vitro transcribed mRNA and a construct. For example, the construct is selected from a plasmid, a retrovirus construct, a lentivirus construct, an adenovirus construct, and an adeno-associated virus (AAV) construct. For example, the nucleic acid comprises a nucleotide sequence which is at least 75% identical, at least 80% identical, at least 85% identical, at least 90% identical, at least 95% identical, at least 98% identical, or at least 99% identical to SEQ ID NO: 39.

For approach (2), at least one shRNA capable of suppressing endogenous Zbtb20 expression is delivered to the T cells using any technique for delivering nucleic acids to mammalian cells, such as use of cationic lipids, viral particles, electroporation, and microinjection. For example, the at least one shRNA is selected from SEQ ID NO: 6, SEQ ID NO: 8, and SEQ ID NO: 10.

Alternatively, a nucleic acid encoding at least one shRNA capable of suppressing endogenous Zbtb20 expression is delivered to the T cells using any technique for delivering nucleic acids to mammalian cells, such as use of cationic lipids, viral particles, electroporation, and microinjection. The nucleic acid is any nucleic acid suitable for expressing at least one shRNA in a mammalian cell. For example, the nucleic acid is a construct selected from a plasmid, a retrovirus construct, a lentivirus construct, an adenovirus construct, and an adeno-associated virus (AAV) construct. For example, the nucleic acid comprises a nucleotide sequence selected from SEQ ID NO: 5, SEQ ID NO: 7, and SEQ ID NO: 9.

For approach (3), at least one sgRNA capable of suppressing endogenous Zbtb20 expression is delivered to the T cells using any technique for delivering nucleic acids to mammalian cells, such as use of cationic lipids, viral particles, electroporation, and microinjection. The at least one sgRNA is capable of binding to at least a portion of the Zbtb20 gene. For example, the at least one sgRNA is selected from SEQ ID NO: 18, SEQ ID NO: 20, SEQ ID NO: 22, and SEQ ID NO: 24. A protein capable of binding to the sgRNA and to a Zbtb20 gene portion, and further capable of cleaving at least one DNA strand of the Zbtb20 gene portion, is also delivered to the T cells using any technique for delivering proteins to mammalian cells. For example, the protein is selected from a Cas9 and Cpf1 (Cas12a). For example, the at least one sgRNA and the protein are delivered to the T cells together as a riboprotein complex using, for example, a cationic lipid.

Alternatively, at least one nucleic acid encoding at least one sgRNA capable of suppressing endogenous Zbtb20 expression is delivered to the T cells using any technique for delivering nucleic acids to mammalian cells, such as use of cationic lipids, viral particles, electroporation, and microinjection. The at least one sgRNA is capable of binding to at least a portion of the Zbtb20 gene. For example, the at least one sgRNA is encoded by a nucleic acid comprising a nucleotide sequence selected from SEQ ID NO: 17, SEQ ID NO: 19, SEQ ID NO: 21, and SEQ ID NO: 23. A nucleic acid encoding a protein capable of binding to the sgRNA and to a Zbtb20 gene portion, and further capable of cleaving at least one DNA strand of the Zbtb20 gene portion, is also delivered to the T cells using any technique for delivering nucleic acids to mammalian cells. For example, the protein is selected from a Cas9 and Cpf1 (Cas12a). For example, the nucleic acid encoding at least one sgRNA and the nucleic acid encoding the protein are the same nucleic acid, for example, a retroviral construct, that is delivered to the T cells within a retroviral particle.

Alternatively, at least one sgRNA capable of suppressing endogenous Zbtb20 expression is delivered to the T cells using any technique for delivering nucleic acids to mammalian cells, such as use of cationic lipids, viral particles, electroporation, and microinjection. The at least one sgRNA is capable of binding to at least a portion of the Zbtb20 promoter, wherein the Zbtb20 promoter portion comprises DNA sequences within, encompassing, and/or close to a Zbtb20 promoter. For example, the at least one sgRNA is selected from SEQ ID NO: 34, SEQ ID NO: 36, and SEQ ID NO: 38. A protein capable of binding to the sgRNA and to a Zbtb20 promoter portion is also delivered to the T cells using any technique for delivering proteins to mammalian cells. For example, the protein is selected from a Cas9 and Cpf1 (Cas12a). For example, the at least one sgRNA and the protein are delivered to the T cells together as a riboprotein complex using, for example, a cationic lipid.

Alternatively, at least one nucleic acid encoding at least one sgRNA capable of suppressing endogenous Zbtb20 expression is delivered to the T cells using any technique for delivering nucleic acids to mammalian cells, such as use of cationic lipids, viral particles, electroporation, and microinjection. The at least one sgRNA is capable of binding to at least a portion of the Zbtb20 promoter, wherein the Zbtb20 promoter portion comprises DNA sequences within, encompassing, and/or close to a Zbtb20 promoter. For example, the at least one sgRNA is encoded by a nucleic acid comprising a nucleotide sequence selected from SEQ ID NO: 33, SEQ ID NO: 35, and SEQ ID NO: 37. A nucleic acid encoding a protein capable of binding to the sgRNA and to a Zbtb20 promoter portion is also delivered to the T cells using any technique for delivering nucleic acids to mammalian cells. For example, the protein is selected from a Cas9 and Cpf1 (Cas12a). For example, the nucleic acid encoding at least one sgRNA and the nucleic acid encoding the protein are the same nucleic acid, for example, a retroviral construct, that is delivered to the T cells within a retroviral particle.

The T cells are optionally further modified to express an exogenous TCR or a CAR. The T cells are further modified to express the exogenous TCR or the CAR prior to or after the T cells are modified to suppress Zbtb20 expression and/or activity. A nucleic acid encoding an exogenous TCR or a CAR, such as a lentiviral construct, can be delivered to the cells. Alternatively, any genetic engineering technique can be used to further modify the T cells such that they express an exogenous TCR or CAR. For example, the genetic engineering approach is selected from a CRISPR/Cas-based genetic engineering method, a TALEN-based genetic engineering method, a ZF-nuclease genetic engineering method, and a transposon-based genetic engineering method.

The subject optionally receives an additional cancer therapy prior to, simultaneously with, and/or after reinfusion of the T cells. The optional additional cancer therapy is selected from immunotherapy, chemotherapy, targeted therapy, stem cell transplant, radiation, surgery, and hormone therapy. The optional immunotherapy is selected from immune checkpoint inhibitors (e.g., negative checkpoint blockade), monoclonal antibodies, cancer vaccines, immune system modulators, and adoptive cell therapies including CAR T-cell therapy, exogenous TCR therapy, and TIL therapy.

An effective amount of the modified T cells is then administered to the subject. The amount of cancer cells in the subject is reduced and/or eliminated following administration of the modified T cells into the subject.

### Example 11: Single Cell Transcriptomic Analysis Shows Enrichment in Metabolic and Memory Pathways in the Absence of Zbtb20

Many studies have shown there is substantial heterogeneity in the CD8 T cell response with respect to the potential to differentiate into memory cells. In order to conduct transcriptomic analyses that could capture this heterogeneity, we performed single cell RNAseq analysis on OT-I cells during the primary response. Using the OT-I transfer, LM-actA-Ova infection model described, WT and Zbtb20 KO CD8 T cells were purified, and CITEseq performed with oligonucleotide-labeled antibodies against KLRG-1, CD127 and CD62L, to orient gene expression patterns with known effector/memory markers.

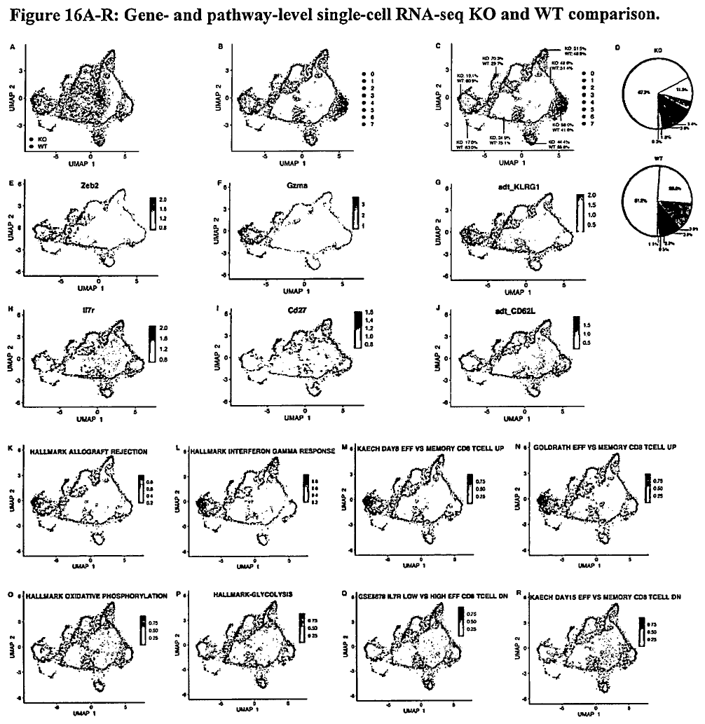

UMAP plots showed some overlaps in clusters occupied by WT and KO cells (FIG. 16A-C), however there were also regions where there was little overlap. In particular a higher proportion of WT cells were in clusters 1 and 2 whereas clusters 0 and 3 were more highly represented in KO cells (FIG. 16B-16C). Analysis of gene representation in these clusters showed that clusters 1 and 2 were enriched for genes and proteins associated with effector T cells (Zeb2, Granzyme A and KLRG-1 staining) (FIG. 16E-G). In contrast, memory associated genes and proteins (IL7r, Cd27 and CD62L staining) were not present in these clusters, and instead seen preferentially in clusters 0, 3, and 5 (FIG. 16H-J), where the majority of KO cells were located. Examination of a wider array of genes expressed in these clusters showed preferential expression of genes associated with effector activity in clusters 1 and 2 (Zeb2, CX3CR1, Klrg1, Gzmb, Gzma) (Gerlach, C., E. A. et al., 2016, Immunity 45: 1270-1284; Böttcher, J. P., et al., 2015, Nat Commun 6: 8306; Hudson, W. H., et al., 2019, Immunity 51: 1043-1058.e4; Omilusik, K. D., et al., 2015, J. Exp. Med. 212: 2027-2039; Dominguez, C. X., et al., 2015, J. Exp. Med. 212: 2041-2056) (FIG. 17A, left panel). Comparison of genes differentially regulated between WT and KO samples showed KO cells expressed higher levels of Pkm and mt-Nd3, necessary for pyruvate synthesis in glycolysis and mitochondrial NADH dehydrogenase, respectively (FIG. 17A, right panel). An extended list of metabolism-associated genes that were differentially expressed is shown in FIG. 17C.

Pathway level analyses were performed using the novel variance-adjusted Mahalanobis method (VAM)(Frost, H. R., 2020, Nucleic Acids Res 48(16):e94) that was recently developed in order to compute cell level gene-set scores visualized in the UMAP plots. Differentially active pathways were also computed using a rank-sum test. Cluster 2 was associated with gene sets previously shown to be upregulated in effector T cells, in addition to gene sets from pro-inflammatory conditions such as allograft rejection and the interferon gamma response (FIG. 16K-N). Gene sets associated with oxidative phosphorylation and glycolysis were preferentially associated with clusters 0 and 3, where the majority of KO cells were located (FIG. 16O-P). A similar pattern of association with clusters 0 and 3 was seen with gene sets previously shown to be downregulated in effector CD8⁺ T cells relative to memory or memory precursor cells (FIG. 16Q-R). An extended list of pathways differentially expressed in the various clusters is shown in FIG. 17B (left panel), and was consistent with effector-associated pathway enrichment in clusters 1 and 2, and memory, glycolysis and mitochondrial metabolism associated pathway enrichment in clusters 0 and 3. Comparison of pathways enriched in KO vs WT samples (FIG. 17B, right panel) showed glycolysis and mitochondrial metabolism associated pathways enriched in KO samples. Pathways upregulated in memory cells when compared with either effector or naïve cells were also enriched KO compared with WT samples. In contrast, effector-associated pathways were enriched in WT samples.

These data clearly confirm our flow cytometric and Seahorse data, showing in the absence of Zbtb20, the CD8⁺ T cell response skews toward the memory phenotype, with enhancement of both glycolytic and mitochondrial metabolism.

### Example 12: Zbtb20 Deficient CD8⁺ T Cells Provide Increased Protection Against B16 Melanoma

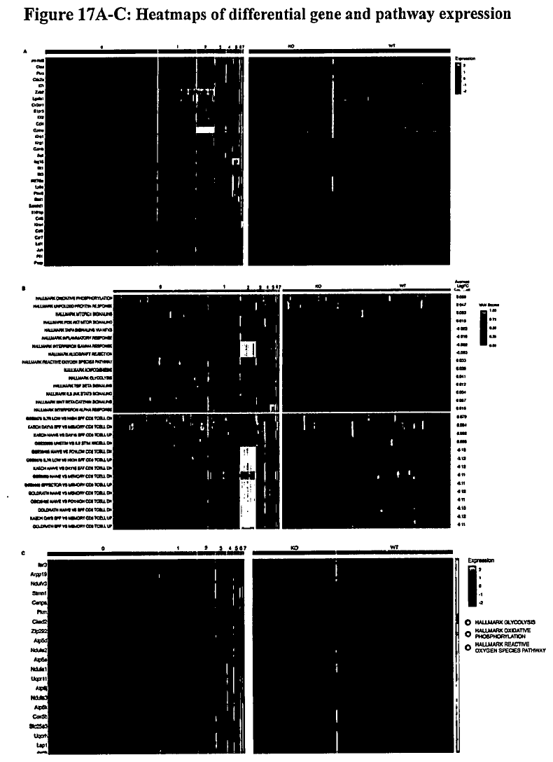

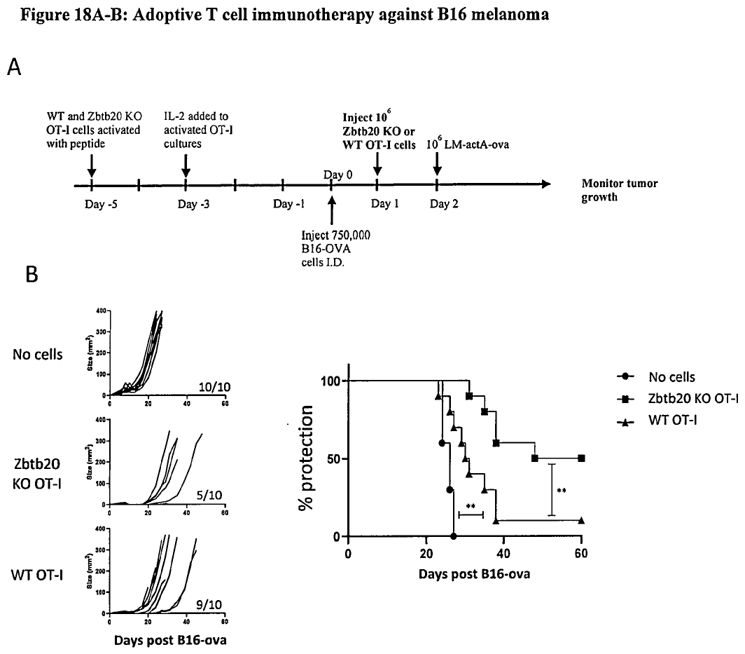

Most adoptive immunotherapy approaches involve in vitro stimulation of T cells prior to transfer into the host bearing a tumor. To model the efficacy of Zbtb20 deficient CD8⁺ T cells in this scenario, we stimulated OT-I cells from naïve WT or Zbtb20 KO mice in vitro, then adoptively transferred these cells into mice bearing B16-ova melanoma as shown in FIG. 18A. One day after T cell transfer, mice were immunized with Listeria monocytogenes-ova to boost the transferred T cells. While WT OT-I cells significantly slowed the growth of B16-ova, 9/10 mice ultimately succumbed within 60 days (FIG. 18B). In contrast B16-ova growth was markedly slower in Zbtb20 KO OT-I recipients, and only 5/10 mice succumbed within 60 days. Therefore, in an adoptive immunotherapy model using in vitro stimulated T cells, Zbtb20 deficient T cells provided better protection against melanoma compared with Zbtb20 sufficient T cells.

### Example 13: Higher Accumulation of Zbtb20 Deficient T Cells in the Tumor, Accompanied by Reduced Upregulation of PD-1

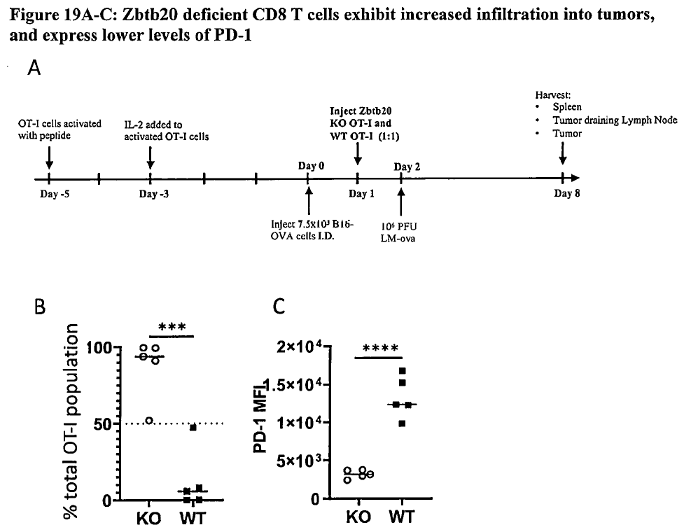

To address the reasons why Zbtb20 deficient CD8⁺ T cells conferred superior protection when compared with WT cells, we measured accumulation of these cells in the tumor. WT and Zbtb20 KO OT-I cells were activated in vitro, then mixed at a 1:1 ratio before being injected into B16-ova bearing mice (FIG. 19A). The tumor infiltrating OT-I population was dominated by Zbtb20 deficient cells and was a significantly larger proportion of the population compared with WT cells (FIG. 19B). PD-1 is an important co-inhibitory molecule that limits T cell function in tumors, therefore we measured PD-1 expression on tumor infiltrating OT-I cells. Expression of PD-1 was significantly decreased on Zbtb20 deficient OT-I cells, when compared with their WT counterparts (FIG. 19C). Therefore Zbtb20 KO CD8⁺ T cells have an enhanced ability to accumulate in the tumor and exhibit lower expression of PD-1, both of which may be associated with their improved anti-tumor activity.

### CONCLUSIONS

Based on phenotypic, functional and metabolic techniques, in conjunction with transcriptional profiling, we have shown that the absence of Zbtb20 skews CD8⁺ T cell differentiation toward the generation of memory. Interestingly, it seems not all KO memory precursor cells survived, as we did not consistently see a larger memory population in KO mice. Bias away from an effector-type profile was particularly evident in our single cell RNAseq analyses, which also showed enrichment for genes sets associated with memory. Both glycolytic and mitochondrial metabolism were enhanced, whereas typically perturbations that promote memory differentiation enhance mitochondrial metabolism at the expense of glycolytic metabolism (Saibil, S. D., et al., 2019, Cancer Res. 79: 445-451; Sukumar, M., et al., 2013, J Clin Invest 123: 4479-4488; Hermans, D., S. et al., 2020, PNAS 117: 6047-6055; Loschinski, R., M. et al., 2018, Oncotarget 9: 13125-13138).

Previous studies have shown a critical role for Zbtb20 in hippocampal development and the correct development of neuronal layers in the cerebral cortex (Nielsen, J. V, et al., 2007, Development 134: 1133-1140; Tonchev, A. B., et al., 2016, Mol. Brain 9(1):65; Rosenthal, E. H., et al., 2012, 22(11): 2144-2156; Xie, Z., et al., 2010, Proc Natl Acad Sci 107: 6510-6515). Consistent with this, patients with certain mutations in Zbtb20 develop Primrose syndrome (Cordeddu, V., B. et al., 2014 Nat Genet. 46: 815-817) which symptoms include intellectual disability, macrocephaly and increased height and weight (Primrose, D. A. et al., 1982, Journal of Mental Deficiency Research, 26(2), 101-106; Mathijssen, I. B., et al., 2005, EurJ Med. Genet. 49: 127-133; Lindor, N. M., et al., 1996, Clin Dysmorphol 5: 27-34; Dalal, P., N. D. et al., 2010, Neurology, 75: 284-28; Collacott, R. A. et al., 1986, J Ment Defic Res. 30 (Pt 3): 301-308; and Battisti, C., M. T. et al., 2002, J Neurology 249: 1466-1468).

Detailed study of patients with Primrose syndrome revealed metabolic changes, including reduced glucose tolerance, with prevalence of amino acid and fatty acid catabolism, ketogenesis, and gluconeogenesis (Casertano, A., P. et al., 2017, JAm Med Genet. 173: 1896-1902). This indicates impairment in the normal pathway from glucose to pyruvate and then into the citric acid cycle. Instead, amino acids and fatty acids are converted to glucose and ketone bodies, similar to the processes that occur in diabetes and during prolonged fasting. This further indicates that Zbtb20 regulates genes are associated with glucose and fatty acid metabolism in humans. Consistent with this, data from Zbtb20 knockout mice showed disrupted glucose homeostasis, and dysreglation of genes associated with glucose metabolism in the liver (Sutherland, A. P. R., et al., 2009, Molecular and Cellular Biology 29: 2804-2815). These mice had severe growth defects and decreased survival, not living beyond 12 weeks of age, however restoration of Zbtb20 selectively in the liver markedly improved survival. Later work using liver-specific Zbtb20 deletion showed Zbtb20 regulates genes associated with glycolysis and de novo lipogenesis (Liu, G., L. et al., 2017, Nat Commun. 8: 14824), and beta-cell specific Zbtb20 deletion lead to aberrant glucose metabolism and altered expression of glycolysis-associated genes (Liu, G., L. et al., 2017, Nat Commun. 8: 14824). To our knowledge, we are the first to describe a role for Zbtb20 in metabolic control in the immune system. Our single cell RNAseq data also suggest that genes central to glycolysis and mitochondrial metabolism are regulated by Zbtb20, and these genes may represent direct or indirect targets of Zbtb20.

It is clear that activated and quiescent T cells have distinct bioenergetic and biosynthetic demands (Pearce, E. L. et al., 2010, Current Opinion in Immunology 22(3):314-20). Activation, proliferation, epigenetic, cytotoxic functions and differentiation of T cells are directed by dynamic changes of their metabolism (Dimeloe, S., A. V. et al., 2017, Immunology 150(1):35-44.). These changes are evident both in mitochondrial structure and in the choice of predominantly mitochondrial or glycolytic metabolism used by the T cell. Mitochondria have a highly compartmentalized structure and their morphology can be very dynamic. Mitochondrial morphology is critical for DNA sequestration, reactive oxygen species regulation, oxidative phosphorylation and calcium homeostasis (Gomes, L. C., G. et al., 2011, Nature Cell Biology 13(5):589-98; Proceedings of the National Academy of Sciences 108(25):10190-5; Vafai, S. B., and V. K. Mootha, 2012, Nature 491(7424):374-83; Mitra, K., C. Et al., 2009, Proceedings of the National Academy of Sciences of the United States of America 106(29):11960-5; Rossignol, R., et al., 2004, Cancer Research 64(3):985-93; Tondera, D., S. et al., 2009, EMBO Journal 28(11):1589-600; and Rambold, A. S., et al., 2015, Developmental Cell; 32(6):678-92), whereas globular and fragmented mitochondria are linked to nutrient excess, lower demand for ATP or severe cellular stress (Jheng, H.-F. et al., 2012, Molecular and Cellular Biology 32(2):309-1; Rambold, A. S., and E. L. Pearce. 2018, Trends in Immunology 39(1):6-18).

Mitochondria can adapt their morphology under different cellular activation states in T cells, macrophages and mast cells (Buck, M. D. D., et al., 2016, Cell 166(1):63-76; Zhou, R., A. S. et al., 2011, Nature 469(7329):221-5; Zhang, B., K. D. et al., 2011, Journal of Allergy and Clinical Immunology 127(6): 1522-31). Rapidly proliferating effector CD8⁺ T cells possess globular mitochondria, whereas memory CD8⁺ T cells contain highly inter-connected, tubular mitochondria (Buck, M. D. D. et al., 2016, Cell 106(1): 63-76) As memory CD8⁺ T cells rely upon mitochondrial respiration for their energy demands, elongated mitochondria with well-ordered cristae are thought to hold components of the electron transport chain in a more efficient configuration (Cogliati, S., C. et al., 2013, Cell 155: 160-171).

Our data indicate that mitochondria in Zbtb20 KO memory CD8⁺ T cells have a larger volume and surface area compared with wild-type cells, which is consistent with enhanced oxidative phosphorylation observed in these cells. Interestingly, mitochondrial content was lower in Zbtb20 KO in vitro-derived effector CD8⁺ T cells. This is also consistent with the observed lower basal and maximal oxidative phosphorylation. Nevertheless KO effector cells did not exhibit impairments in cytokine production or proliferation, presumably due to the enhanced glycolytic metabolism we observed, which provided the necessary ATP and biosynthetic intermediates.

Our Seahorse assays clearly showed Zbtb20 deficiency modulates T cell metabolism, however there were some subtle differences observed between in vitro and ex vivo generated effector and memory cells. Basal and maximal glycolysis and oxidative phosphorylation were uniformly increased in ex vivo effector and memory CD8⁺ T cells. While IL-15 generated memory cells also displayed elevated basal and maximal oxidative phosphorylation, glycolytic parameters were similar to wild-type cells. Effector CD8⁺ T cells generated with IL-2 had elevated basal, but not maximal glycolysis, but depressed basal and maximal oxidative phosphorylation. Several factors may be responsible for these discrepancies. CD8⁺ T cells responding to an infection in lymph nodes or the spleen are exposed to a variety of pro-inflammatory mediators, cytokines and activated antigen-presenting cells that are not faithfully replicated by standard in vitro culture conditions. In addition concentrations of key nutrients such as glucose and glutamate are in excess in vitro, and likely more limiting in vivo (Ma, E. H., M et al., 2019, Immunity 51: 856-870.e5). A recent study found in vitro-derived effector cells operated at their maximal glycolytic capacity, whereas ex vivo-derived cells had larger spare energetic capacity (Ma et al., (Id.). Ex vivo cells also displayed greater oxidative metabolism and switched more easily between mitochondrial and glycolytic pathways. Therefore it is possible the increased metabolic flexibility in Zbtb20 KO cells, possibly in addition to exposure to inflammatory factors present uniquely in vivo, results in the metabolic changes in these cells being better revealed in vivo.

Effector CD8⁺ T cells heavily rely on glycolysis and have high rates of glucose uptake (25), whereas memory CD8⁺ T cells rely on mitochondrial respiration (Pearce, E. L. et al., 2010, Current Opinion in Immunology 22(3): 314-320). It is clear that the substrate used in the mitochondrial citric acid cycle also influences CD8⁺ T cell function, differentiation and longevity (Dimeloe, S., A. V. et al., 2017, Immunology 150(1):35-44). Glutamine metabolism has been reported to be crucial for survival, proliferation and effector function of CD4 T cells upon activation (Nakaya, M., et al., 2014, Immunity 40(5):692-705.). Fatty acid oxidation has been linked to superior mitochondrial capacity and longevity of memory CD8⁺ T cells (van der Windt, G. J. W., et al., 2012, Immunity 36: 68-78; O'Sullivan, D., et al., 2014, Immunity 41(1):75-88). In addition, instead of obtaining fatty acids from their external environment, memory CD8⁺ T cell synthesize their own triacylglycerol using glucose-derived carbon (O'Sullivan, D., et al., 2014, Immunity 41(1):75-88; Cui, G., M. M., et al., 2015, Cell 161(4):750-61). Concomitantly, memory CD8⁺ T cell also up-regulate expression of the glycerol channel, aquaporin 9, to facilitate the uptake of glycerol required for triacylglycerol synthesis and storage (Cui, G., et al., 2015, Cell 161(4):750-61). Subsequent studies showed that medium or short chain fatty acids such as acetate also play important roles as mitochondrial fuels in memory CD8 T cells (Raud, B., et al., 2018, Cell Metab. 28: 504-515.e7; Balmer, M. L., et al., 2016, Immunity 44: 1312-1324; Bachem, A., C. et al., 2019, Immunity 51: 285-297.e5). Our studies regarding mitochondrial fuel sources show inhibition of glutaminolysis or glycolysis markedly impair mitochondrial respiratory activity in WT CD8⁺ T_{cm} cells. However Zbtb20 deficient memory CD8⁺ T cells tolerated inhibition of either fuel source without significant diminution of mitochondrial respiration. In fact only when both pathways were inhibited was there a significant reduction. Availability of glucose and glutamate are limiting in many growing tumors, creating an environment not conducive for protective T cell responses. Limited flexibility with respect to mitochondrial fuel sources may restrict the protective capacity of WT CD8⁺ T cells, and increased flexibility on the part of Zbtb20 deficient memory cells may partially explain their increased protective capacity.

Spare respiratory capacity is thought to be an important factor contributing to enhanced secondary responses by memory CD8 T cells in response to antigenic rechallenge (van der Windt, G. J. W., et al., 2012, Immunity 36: 68-78). Therefore it is likely that the larger spare respiratory capacity we observed in Zbtb20-deficient memory CD8 T cells is at least partly responsible for the greater secondary expansion following virus re-challenge. Improved protective capacity from Zbtb20 KO memory cells was demonstrated by superior ability to protect against MC38-Ova tumors. While enhanced expansion of memory cells is no doubt important in this protection, a higher proportion of cells expressing effector cytokines such as IFN-γ and TNF-α, and CXCR3, which may promote homing to the tumor site, may also have contributed to anti-tumor activity.

Our data indicates that Zbtb20 is expressed in the first 2-3 days following CD8⁺ T cell activation, and is important in shaping the phenotypic, metabolic and functional evolution of the anti-microbial response. Expression then declines rapidly, but re-emerges in a small subset of memory CD8⁺ T cells. This may indicate that Zbtb20 exerts its effects during the first few days of the T cell response, then is subsequently active in a defined population of memory cells. Early Zbtb20 activity may exert a sustained effect in part through modulation of the network of other transcription factors critical for T cell differentiation. Blimp-1 suppresses effector CD8⁺ T cell proliferation and drives their terminal differentiation, whereas Bcl-6 promotes proliferation, survival and memory differentiation of CD8 T cells (Russ, B. E., et al., 2012, Frontiers in Immunology 3:371). Eomesodermin induces expression of several effector molecules in T cells, such as IFN-γ, perforin and granzyme B (Pearce, E. L., A et al., 2003, Science 302: 1041-1043), but also promotes homeostatic self-renewal of memory cells through inducing expression of the IL-15 receptor (Intlekofer, A. M., et al., 2005, Nature Immunology 6: 1236-1244). Reduced expression of Blimp-1 and Eomes at d7 may contribute to the skewing away from terminally differentiated effector cells and toward memory precursors. Expression of these molecules change during the contraction phase (D14), however this could be a reflection of the altered proportions of effector and memory cells during contraction, as effectors die off and the proportion of memory precursors enlarges. We also observed elevated Bcl-6 expression at day 7, which is consistent with promotion of memory precursor development. However a key function of Bcl-6 is to directly repress genes involved in the glycolysis pathway, including Slc2a1, SIc2a3, Hk2 and Pkm2 (Oestreich, K. J., et al., 2014, Nature Immunology 15(10):957-64). As we observed increased glycolytic metabolism in the absence of Zbtb20, the effects of elevated Bcl-6 were likely mitigated by other transcription factors or cofactors.

While most experiments focused on the CD8 T cell response to listeria infection, we also tested the extent to which they extended to a different, unrelated infection. Murine gammaherpesvirus infection is a different class of pathogen (virus vs intracellular bacteria), and unlike listeria, it establishes a persistent infection (Obar, J. J., S et al., 2006, J Virol 80: 8303-8315). While we detected changes in T cell metabolism and altered expression of key transcription factors in both infections, there were important differences. Glycolysis was increased in Zbtb20 deficient CD8*T cells in both infections. Basal and maximal mitochondrial respiratory capacity and spare respiratory capacity were all enhanced in knockout memory cells in listeria infection, however these changes were of smaller magnitude in MHV-68 infection. The pattern of expression of Bcl-6, Eomes and T-bet were consistent in memory cells in both infections, however they differed at the acute timepoints. There are a number of factors that may be responsible for these differences, including antigen persistence, engagement of different pattern recognition receptors and cellular tropism. Despite these differences, however, it is clear Zbtb20 affects both immunometabolism and the transcriptional network during CD8⁺ T cell differentiation across infection types.

In conclusion, we have proven that Zbtb20 is an important regulator of effector and memory CD8⁺ T cell differentiation and metabolism. Given our data showing improved protection from tumors, and the known superiority of memory cells in adoptive T cell therapy, deletion or inhibition of Zbtb20 provides a novel strategy for anti-tumor immunotherapy.

### Exemplary Sequences

Nucleotide and amino acid sequences provided in this disclosure are in Table 1 below.

TABLE 1
Nucleotide and amino acid sequences

| SEQ | |
|---|---|
| ID NO: | SEQUENCE |
| 1 | Human Zbtb20 cDNA nucleotide sequence: |
| | atgaccgagcgcattcacagcatcaaccttcacaacttcagcaattccgtgctcgagaccctcaacgagcagcgcaaccgt |
| | ggccacttctgtgacgtaacggtgcgcatccacgggagcatgctgcgcgcacaccgctgcgtgctggcagccggcagcccc |
| | ttcttccaggacaaactgctgcttggctacagcgacatcgagatcccgtcggtggtgtcagtgcagtcagtgcaaaagctc |
| | attgacttcatgtacagcggcgtgctacgggtctcgcagtcggaagctctgcagatcctcacggccgccagcatcctgcag |
| | atcaaaacagtcatcgacgagtgcacgcgcatcgtgtcacagaacgtgggcgatgtgttcccggggatccaggactcgggc |
| | caggacacgccgcggggcactcccgagtcaggcacgtcaggccagagcagcgacacggagtcgggctacctgcagagccac |
| | ccacagcacagcgtggacaggatctactcggcactctacgcgtgctccatgcagaatggcagcggcgagcgctctttttac |
| | agcggcgcagtggtcagccaccacgagactgcgctcggcctgccccgcgaccaccacatggaagaccccagctggatcaca |
| | cgcatccatgagcgctcgcagcagatggagcgctacctgtccaccacccccgagaccacgcactgccgcaagcagccccgg |
| | cctgtgcgcatccagaccctagtgggcaacatccacatcaagcaggagatggaggacgattacgactactacgggcagcaa |
| | agggtgcagatcctggaacgcaacgaatccgaggagtgcacggaagacacagaccaggccgagggcaccgagagtgagccc |
| | aaaggtgaaagcttcgactcgggcgtcagctcctccataggcaccgagcctgactcggtggagcagcagtttgggcctggg |
| | gcggcgcgggacagccaggctgaacccacccaacccgagcaggctgcagaagcccccgctgagggtggtccgcagacaaac |
| | cagctagaaacaggtgcttcctctccggagagaagcaatgaagtggagatggacagcactgttatcactgtcagcaacagc |
| | tccgacaagagcgtcctacaacagccttcggtcaacacgtccatcgggcagccattgccaagtacccagctctacttacgc |
| | cagacagaaaccctcaccagcaacctgaggatgcctctgaccttgaccagcaacacgcaggtcattggcacagctggcaac |
| | acctacctgccagccctcttcactacccagcccgcgggcagtggccccaagcctttcctcttcagcctgccacagcccctg |
| | gcaggccagcagacccagtttgtgacagtgttccagcccggtctgtcgacctttactgcacagctgccagcgccacagccc |
| | ctggcctcatccgcaggccacagcacagccagtgggcaaggcgaaaaaaagccttatgagtgcactctctgcaacaagact |
| | ttcaccgccaaacagaactacgtcaagcacatgttcgtacacacaggtgagaagccccaccaatgcagcatctgttggcgc |
| | tccttctccttaaaggattaccttatcaagcacatggtgacacacacaggagtgagggcataccagtgtagtatctgcaac |
| | aagcgcttcacccagaagagctccctcaacgtgcacatgcgcctccaccggggagagaagtcctacgagtgctacatctgc |
| | aaaaagaagttctctcacaagaccctcctggagcgacacgtggccctgcacagtgccagcaatgggaccccccctgcaggc |
| | acacccccaggtgcccgcgctggccccccaggcgtggtggcctgcacggaggggaccacttacgtctgctccgtctgccca |
| | gcaaagtttgaccaaatcgagcagttcaacgaccacatgaggatgcatgtgtctgacgga |
| 2 | Human Zbtb20 amino acid sequence: |
| | MTERIHSINLHNFSNSVLETLNEQRNRGHFCDVTVRIHGSMLRAHRCVLAAGSPFFQDKLLLGYSDIEIPSVVSVQSVQKL |
| | IDFMYSGVLRVSQSEALQILTAASILQIKTVIDECTRIVSQNVGDVFPGIQDSGQDTPRGTPESGTSGQSSDTESGYLQSH |
| | PQHSVDRIYSALYACSMQNGSGERSFYSGAVVSHHETALGLPRDHHMEDPSWITRIHERSQQMERYLSTTPETTHCRKQPR |
| | PVRIQTLVGNIHIKQEMEDDYDYYGQQRVQILERNESEECTEDTDQAEGTESEPKGESFDSGVSSSIGTEPDSVEQQFGPG |
| | AARDSQAEPTQPEQAAEAPAEGGPQTNQLETGASSPERSNEVEMDSTVITVSNSSDKSVLQQPSVNTSIGQPLPSTQLYLR |
| | QTETLTSNLRMPLTLTSNTQVIGTAGNTYLPALFTTQPAGSGPKPFLFSLPQPLAGQQTQFVTVFQPGLSTFTAQLPAPQP |
| | LASSAGHSTASGQGEKKPYECTLCNKTFTAKQNYVKHMFVHTGEKPHQCSICWRSFSLKDYLIKHMVTHTGVRAYQCSICN |
| | KRFTQKSSLNVHMRLHRGEKSYECYICKKKFSHKTLLERHVALHSASNGTPPAGTPPGARAGPPGVVACTEGTTYVCSVCP |
| | AKFDQIEQFNDHMRMHVSDG |
| 3 | Mouse Zbtb20 cDNA nucleotide sequence: |
| | atgctagaacggaagaaacccaagacagctgaaaaccagaaggcatctgaggagaatgagattactcagccgggcggatcc |
| | agcgccaagccggcccttccctgcctgaactttgaagctgttttgtctccagccccagccctcatccactcgacacattca |
| | ctgacaaactctcacgctcacaccgggtcatctgattgtgacatcagttgcaaggggatgaccgagcgcattcacagcatc |
| | aaccttcacaacttcagcaattccgtgctcgagaccctcaacgagcagcgcaaccgtggccacttctgtgacgtgacggtt |
| | cgcatccacgggagcatgctgcgcgcacatcgctgcgtgctggcagccggcagccccttcttccaagacaagctgctgctg |
| | ggctacagcgacatcgaaatcccgtcggtggtgtccgtacaatcggtgcaaaagctcattgacttcatgtacagcggtgtg |
| | ctgagagtctcacagtcggaagctctgcagatcctcacagccgccagcatcctgcagatcaaaacagtcatagatgagtgc |
| | actcgcatcgtgtcacagaacgtgggcgatgtgttcccaggcatccaggattctggccaggacacaccaagaggcacacca |
| | gagtcaggcacatctggccagagcagtgacacggaatcaggctacctgcagagccacccacagcatagtgtggaccgaatc |
| | tactccgcactctacgcctgctccatgcagaatggcagcggcgagcgctccttctacagtggtgcagtggtcagccaccac |
| | gaaacagctctcggcctgccccgtgaccaccacatggaagaccctagctggatcacacgcattcatgagcgctcccagcaa |
| | atggagcgctacctgtccaccacccctgagaccacgcactgccggaagcagccccggcctgtgcgtatccagaccctggtg |
| | ggtaacatccacatcaagcaggaaatggaagatgactatgactactatgggcagcaaagggtgcagatcctagaacgcaat |
| | gaatccgaggagtgcacagaagacactgaccaagcagagggcactgagagcgagcccaaaggtgaaagctttgattctggg |
| | gtcagctcctccatcggcaccgaacctgactcagtggagcaacagtttggggcagcagccccaagggacggtcaggcagaa |
| | cccgcccaacctgagcaggcagcagaagccccagctgagagcagtgcccagccaaaccagctagaaccaggtgcctcctct |
| | cctgagagaagcaacgagtcagagatggacaacacagtcatcactgtcagtaacagctccgataagggcgtcctacagcag |
| | ccttcagtcaacacatccatcgggcagccattgccaagtacccagctctatttacgccagacagaaaccctcaccagcaac |
| | ctgaggatgcctctgaccttgaccagcaacacacaggtcattggcaccgctggcaacacctatctgccagccctcttcact |
| | acccaacccgcgggcagtggccccaagccttttctcttcagcctgccgcagcccctgacaggccagcagacccagtttgtg |
| | acagtgtcccagcccggtctgtccacctttactgcacagctgccagcgccacagcccctggcctcatctgcaggccacagc |
| | acagccagtgggcaaggcgacaaaaagccttatgagtgcactctctgcaacaagactttcacagccaaacagaactacgtc |
| | aagcacatgttcgtacatacaggtgagaagccccaccagtgcagcatctgctggcgctccttctccttgaaggattacctt |
| | atcaagcacatggtgacgcacaccggcgtgagagcgtaccagtgtagcatctgcaacaagcgcttcacccagaagagttcc |
| | ctcaacgtgcacatgcgcctgcaccgcggggagaagtcctatgagtgctacatctgcaaaaagaagttctcccacaagacc |
| | ctgctggagcgacacgtggccctgcacagtgccagcaacgggacccctccggcaggcacgcccccaggtgcccgcgcgggt |
| | ccgccaggcgtggtggcctgcacagaggggaccacttacgtctgctccgtctgcccagcaaagtttgaccaaatcgagcag |
| | ttcaacgaccacatgaggatgcatgtgtctgacgga |
| 4 | Mouse Zbtb20 amino acid sequence: |
| | MLERKKPKTAENQKASEENEITQPGGSSAKPALPCLNFEAVLSPAPALIHSTHSLTNSHAHTGSSDCDISCKGMTERIHSI |
| | NLHNFSNSVLETLNEQRNRGHFCDVTVRIHGSMLRAHRCVLAAGSPFFQDKLLLGYSDIEIPSVVSVQSVQKLIDFMYSGV |
| | LRVSQSEALQILTAASILQIKTVIDECTRIVSQNVGDVFPGIQDSGQDTPRGTPESGTSGQSSDTESGYLQSHPQHSVDRI |
| | YSALYACSMQNGSGERSFYSGAVVSHHETALGLPRDHHMEDPSWITRIHERSQQMERYLSTTPETTHCRKQPRPVRIQTLV |
| | GNIHIKQEMEDDYDYYGQQRVQILERNESEECTEDTDQAEGTESEPKGESFDSGVSSSIGTEPDSVEQQFGAAAPRDGQAE |
| | PAQPEQAAEAPAESSAQPNQLEPGASSPERSNESEMDNTVITVSNSSDKGVLQQPSVNTSIGQPLPSTQLYLRQTETLTSN |
| | LRMPLTLTSNTQVIGTAGNTYLPALFTTQPAGSGPKPFLFSLPQPLTGQQTQFVTVSQPGLSTFTAQLPAPQPLASSAGHS |
| | TASGQGDKKPYECTLCNKTFTAKQNYVKHMFVHTGEKPHQCSICWRSFSLKDYLIKHMVTHTGVRAYQCSICNKRFTQKSS |
| | LNVHMRLHRGEKSYECYICKKKFSHKTLLERHVALHSASNGTPPAGTPPGARAGPPGVVACTEGTTYVCSVCPAKFDQIEQ |
| | FNDHMRMHVSDG |
| 5 | DNA encoding shRNA targeting human Zbtb20 transcript: |
| | CCGGCGCAGACAAACCAGCTAGAAACTCGAGTTTCTAGCTGGTTTGTCTGCGTTTTT |
| 6 | shRNA targeting human Zbtb20 transcript: |
| | CCGGCGCAGACAAACCAGCUAGAAACUCGAGUUUCUAGCUGGUUUGUCUGCGUUUUU |
| 7 | DNA encoding shRNA targeting human Zbtb20 transcript: |
| | CCGGCCCAGCAAAGTTTGACCAAATCTCGAGATTTGGTCAAACTTTGCTGGGTTTTT |
| 8 | shRNA targeting human Zbtb20 transcript: |
| | CCGGCCCAGCAAAGUUUGACCAAAUCUCGAGAUUUGGUCAAACUUUGCUGGGUUUUU |
| 9 | DNA encoding shRNA targeting human Zbtb20 transcript: |
| | CCGGCGGGTCATCTGATTGTGACATCTCGAGATGTCACAATCAGATGACCCGTTTTTG |
| 10 | shRNA targeting human Zbtb20 transcript: |
| | CCGGCGGGUCAUCUGAUUGUGACAUCUCGAGAUGUCACAAUCAGAUGACCCGUUUUUG |
| 11 | DNA encoding shRNA targeting mouse Zbtb20 transcript: |
| | CCGGGGGCTACAGCGACATCGAAATCTCGAGATTTCGATGTCGCTGTAGCCCTTTTTG |
| 12 | shRNA targeting mouse Zbtb20 transcript: |
| | CCGGGGGCUACAGCGACAUCGAAAUCUCGAGAUUUCGAUGUCGCUGUAGCCCUUUUUG |
| 13 | DNA encoding shRNA targeting mouse Zbtb20 transcript: |
| | CCGGGCCTGCTGGTACATTACATTTCTCGAGAAATGTAATGTACCAGCAGGCTTTTTG |
| 14 | shRNA targeting mouse Zbtb20 transcript: |
| | CCGGGCCUGCUGGUACAUUACAUUUCUCGAGAAAUGUAAUGUACCAGCAGGCUUUUUG |
| 15 | DNA encoding shRNA targeting mouse Zbtb20 transcript: |
| | CCGGAGCTATGGCACTAGAATTTAACTCGAGTTAAATTCTAGTGCCATAGCTTTTTTG |
| 16 | shRNA targeting mouse Zbtb20 transcript: |
| | CCGGAGCUAUGGCACUAGAAUUUAACUCGAGUUAAAUUCUAGUGCCAUAGCUUUUUUG |
| 17 | DNA encoding sgRNA targeting human Zbtb20 gene: |
| | GTTGATGCTGTGAATGCGCT |
| 18 | sgRNA targeting human Zbtb20 gene: |
| | GUUGAUGCUGUGAAUGCGCU |
| 19 | DNA encoding sgRNA targeting human Zbtb20 gene: |
| | CGGAATTGCTGAAGTTGTGA |
| 20 | sgRNA targeting human Zbtb20 gene: |
| | CGGAAUUGCUGAAGUUGUGA |
| 21 | DNA encoding sgRNA targeting human Zbtb20 gene: |
| | CTCGTTGAGGGTCTCGAGCA |
| 22 | sgRNA targeting human Zbtb20 gene: |
| | CUCGUUGAGGGUCUCGAGCA |
| 23 | DNA encoding sgRNA targeting human Zbtb20 gene: |
| | ACGGTTGCGCTGCTCGTTGA |
| 24 | sgRNA targeting human Zbtb20 gene: |
| | ACGGUUGCGCUGCUCGUUGA |
| 25 | DNA encoding sgRNA targeting mouse Zbtb20 gene: |
| 26 | sgRNA targeting mouse Zbtb20 gene: |
| | CAAGACAGCUGAAAACCAGA |
| 27 | DNA encoding sgRNA targeting mouse Zbtb20 gene: |
| | TGAAAACCAGAAGGCATCTG |
| 28 | sgRNA targeting mouse Zbtb20 gene: |
| | UGAAAACCAGAAGGCAUCUG |
| 29 | DNA encoding sgRNA targeting mouse Zbtb20 gene: |
| | GGAGAATGAGATTACTCAGC |
| 30 | sgRNA targeting mouse Zbtb20 gene: |
| | GGAGAAUGAGAUUACUCAGC |
| 31 | DNA encoding sgRNA targeting mouse Zbtb20 gene: |
| | GAGAATGAGATTACTCAGCC |
| 32 | sgRNA targeting mouse Zbtb20 gene: |
| | GAGAAUGAGAUUACUCAGCC |
| 33 | DNA encoding sgRNA targeting human Zbtb20 promoter: |
| | ACTTACTCTTTCTGCTCGGG |
| 34 | sgRNA targeting human Zbtb20 promoter: |
| | ACUUACUCUUUCUGCUCGGG |
| 35 | DNA encoding sgRNA targeting human Zbtb20 promoter: |
| | CCAGCATGAGCTGGAAATGT |
| 36 | sgRNA targeting human Zbtb20 promoter: |
| | CCAGCAUGAGCUGGAAAUGU |
| 37 | DNA encoding sgRNA targeting human Zbtb20 promoter: |
| | CGGTACAGTCCAGCATGAGC |
| 38 | sgRNA targeting human Zbtb20 promoter: |
| | CGGUACAGUCCAGCAUGAGC |
| 39 | Human dominant negative Zbtb20 cDNA nucleotide sequence: |
| | atgctgccacagcccctggcaggccagcagacccagtttgtgacagtgttccagcccggtctgtcgacctttactgcacag |
| | ctgccagcgccacagcccctggcctcatccgcaggccacagcacagccagtgggcaaggcgaaaaaaagccttatgagtgc |
| | actctctgcaacaagactttcaccgccaaacagaactacgtcaagcacatgttcgtacacacaggtgagaagccccaccaa |
| | tgcagcatctgttggcgctccttctccttaaaggattaccttatcaagcacatggtgacacacacaggagtgagggcatac |
| | cagtgtagtatctgcaacaagcgcttcacccagaagagctccctcaacgtgcacatgcgcctccaccggggagagaagtcc |
| | tacgagtgctacatctgcaaaaagaagttctctcacaagaccctcctggagcgacacgtggccctgcacagtgccagcaat |
| | gggaccccccctgcaggcacacccccaggtgcccgcgctggccccccaggcgtggtggcctgcacggaggggaccacttac |
| | gtctgctccgtctgcccagcaaagtttgaccaaatcgagcagttcaacgaccacatgaggatgcatgtgtctgacgga |
| 40 | Human dominant negative Zbtb20 amino acid sequence: |
| | MLPQPLAGQQTQFVTVFQPGLSTFTAQLPAPQPLASSAGHSTASGQGEKKPYECTLCNKTFTAKQNYVKHMFVHTGEKPHQ |
| | CSICWRSFSLKDYLIKHMVTHTGVRAYQCSICNKRFTQKSSLNVHMRLHRGEKSYECYICKKKFSHKTLLERHVALHSASN |
| | GTPPAGTPPGARAGPPGVVACTEGTTYVCSVCPAKFDQIEQFNDHMRMHVSDG |
| 41 | Mouse dominant negative Zbtb20 cDNA nucleotide sequence: |
| | atgctgccgcagcccctgacaggccagcagacccagtttgtgacagtgtcccagcccggtctgtccacctttactgcacag |
| | ctgccagcgccacagcccctggcctcatctgcaggccacagcacagccagtgggcaaggcgacaaaaagccttatgagtgc |
| | actctctgcaacaagactttcacagccaaacagaactacgtcaagcacatgttcgtacatacaggtgagaagccccaccag |
| | tgcagcatctgctggcgctccttctccttgaaggattaccttatcaagcacatggtgacgcacaccggcgtgagagcgtac |
| | cagtgtagcatctgcaacaagcgcttcacccagaagagttccctcaacgtgcacatgcgcctgcaccgcggggagaagtcc |
| | tatgagtgctacatctgcaaaaagaagttctcccacaagaccctgctggagcgacacgtggccctgcacagtgccagcaac |
| | gggacccctccggcaggcacgcccccaggtgcccgcgcgggtccgccaggcgtggtggcctgcacagaggggaccacttac |
| | gtctgctccgtctgcccagcaaagtttgaccaaatcgagcagttcaacgaccacatgaggatgcatgtgtctgacgga |
| 42 | Mouse dominant negative Zbtb20 amino acid sequence: |
| | MLPQPLTGQQTQFVTVSQPGLSTFTAQLPAPQPLASSAGHSTASGQGDKKPYECTLCNKTFTAKQNYVKHMFVHTGEKPHQ |
| | CSICWRSFSLKDYLIKHMVTHTGVRAYQCSICNKRFTQKSSLNVHMRLHRGEKSYECYICKKKFSHKTLLERHVALHSASN |
| | GTPPAGTPPGARAGPPGVVACTEGTTYVCSVCPAKFDQJEQFNDHMRMHVSDG |

We claim:

1. A method for treating a subject with a cancer or precancer or a subject at increased risk of developing cancer, comprising administering an effective amount of cells to the subject, wherein the cells are modified ex vivo to suppress endogenous Zbtb20 expression and/or activity within the modified cells.

2. The method of claim 1, wherein the subject is at increased risk of developing cancer because of one or more of (i) a genetic risk factor, (ii) expression or aberrant expression of at least one biomarker correlated to cancer, (iii) a previous cancer.

3. A method of inhibiting Zbtb20 expression and/or activity, wherein such method prevents or inhibits PD-1 upregulation, wherein Zbtb20 expression inhibition and/or activity is optionally effected by administering an effective amount of cells to the subject, wherein the cells are modified ex vivo to suppress endogenous Zbtb20 expression and/or activity within the modified cells.

4. The method of claim 3 which prevents or inhibits T cell exhaustion in adoptive immunotherapy, optionally adoptive immunotherapy for the treatment of cancer or an infectious condition.

5. The method of any of claims 1-4, wherein the modified cells comprise immune cells.

6. The method of any one of the foregoing claims, wherein the modified cells comprise autologous immune cells.

7. The method of any one of the foregoing claims, wherein the modified cells comprise allogenic immune cells.

8. The method of any one of the foregoing claims, wherein the modified cells comprise T cells and/or T cell progenitors.

9. The method of any one of the foregoing claims, wherein the modified cells comprise NK cells.

10. The method of any one of the foregoing claims, wherein the modified cells comprise CD8⁺ T cells and/or CD8⁺ T cell progenitors.

11. The method of any one of the foregoing claims, wherein the modified cells comprise CD4⁺ T cells and/or CD4⁺ T cell progenitors.

12. The method of any one of the foregoing claims, wherein the immune cells comprise lymphocytes, T cells, NK cells, B cells, neutrophils (granulocytes), monocytes, and/or dendritic cells.

13. The method of any one of the foregoing claims, wherein the modified cells are mammalian cells selected from rodent cells, non-human primate cells, and human cells.

14. The method of any one of the foregoing claims, wherein the subject is a mammal selected from a rodent, a non-human primate, and a human.

15. The method of any one of the foregoing claims, wherein the modified cells comprise a dominant negative Zbtb20,
wherein the dominant negative Zbtb20 comprises one or more Zbtb20 C-terminal zinc-finger domains and lacks at least a portion of a Zbtb20 N-terminal region comprising a Zbtb20 BTB domain; and
wherein the dominant negative Zbtb20 suppresses endogenous Zbtb20 activity within the modified cells.

16. The method of any one of the foregoing claims, wherein the dominant negative Zbtb20 is encoded by a nucleic acid comprising a nucleotide sequence which is at least 75% identical, at least 80% identical, at least 85% identical, at least 90% identical, at least 95% identical, at least 98% identical, or at least 99% identical to SEQ ID NO: 39 or SEQ ID NO: 41 or to another mammalian Zbtb20 coding sequence.

17. The method of any one of the foregoing claims, wherein the nucleic acid encoding the dominant negative Zbtb20 is a construct comprising at least one promoter,
wherein the at least one promoter is operatively linked to the nucleotide sequence; and
wherein the promoter is selected from a constitutive promoter and an inducible promoter.

18. The method of any one of the foregoing claims, wherein the construct is selected from a plasmid, a retrovirus construct, a lentivirus construct, an adenovirus construct, and an adeno-associated virus (AAV) construct.

19. The method of any one of the foregoing claims, wherein the nucleic acid encoding the dominant negative Zbtb20 is an in vitro transcribed mRNA.

20. The method of any one of the foregoing claims, wherein the dominant negative Zbtb20 and/or the nucleic acid encoding the dominant negative Zbtb20 is delivered to the modified cells prior to the administration of the modified cells to the subject.

21. The method of any of the foregoing claims, wherein the modified cells are genetically engineered to express the dominant negative Zbtb20 prior to the administration of the modified cells to the subject, wherein the genetic engineering comprises a CRISPR/Cas-based genetic engineering method, a TALEN-based genetic engineering method, a ZF-nuclease genetic engineering method or a transposon-based genetic engineering method.

22. The method of any one of the foregoing claims, wherein the dominant negative Zbtb20 comprises an amino acid sequence which is at least 75% identical, at least 80% identical, at least 85% identical, at least 90% identical, at least 95% identical, at least 98% identical, or at least 99% identical to SEQ ID NO: 40 or SEQ ID NO: 42 or to another mammalian Zbtb20 amino acid sequence.

23. The method of any one of the foregoing claims, wherein the modified cells comprise at least one non-coding RNA capable of suppressing endogenous Zbtb20 expression in the modified cells.

24. The method of any one of the foregoing claims, wherein the at least one non-coding RNA comprises at least one shRNA capable of suppressing endogenous Zbtb20 expression in the modified cells.

25. The method of any one of the foregoing claims, wherein the at least one shRNA is encoded by a nucleic acid comprising a nucleotide sequence selected from SEQ ID NO: 5, SEQ ID NO: 7, SEQ ID NO: 9, SEQ ID NO: 11, SEQ ID NO: 13, and SEQ ID NO: 15.

26. The method of any one of the foregoing claims, wherein the nucleic acid encoding at least one shRNA is a construct comprising at least one promoter,
wherein the at least one promoter is operatively linked to the nucleotide sequence; and
wherein the promoter is selected from a constitutive promoter and an inducible promoter.

27. The method of any one of the foregoing claims, wherein the construct is selected from a plasmid, a retrovirus construct, a lentivirus construct, an adenovirus construct, and an adeno-associated virus (AAV) construct.

28. The method of any one of the foregoing claims, wherein the at least one shRNA and/or the nucleic acid encoding at least one shRNA is delivered to the modified cells prior to the administration of the modified cells to the subject.

29. The method of any one of the foregoing claims, wherein the at least one shRNA is selected from SEQ ID NO: 6, SEQ ID NO: 8, SEQ ID NO: 10, SEQ ID NO: 12, SEQ ID NO: 14, and SEQ ID NO: 16.

30. The method of any one of the foregoing claims, wherein the at least one non-coding RNA comprises at least one sgRNA capable of suppressing endogenous Zbtb20 expression in the modified cells.

31. The method of any one of the foregoing claims, wherein the at least one sgRNA is encoded by a nucleic acid comprising a nucleotide sequence selected from SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

32. The method of any one of the foregoing claims, wherein the nucleic acid encoding at least one sgRNA is a construct comprising at least one promoter,
wherein the at least one promoter is operatively linked to the nucleotide sequence; and
wherein the promoter is selected from a constitutive promoter and an inducible promoter.

33. The method of any one of the foregoing claims, wherein the construct is selected from a plasmid, a retrovirus construct, a lentivirus construct, an adenovirus construct, and an adeno-associated virus (AAV) construct.

34. The method of any one of the foregoing claims, wherein the at least one sgRNA and/or the nucleic acid encoding at least one sgRNA is delivered to the modified cells prior to the administration of the modified cells to the subject.

35. The method of any one of the foregoing claims, wherein the at least one sgRNA is selected from SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, and SEQ ID NO: 38.

36. The method of any one of the foregoing claims, wherein the modified cells further comprise a protein capable of binding to the sgRNA and to at least one of a Zbtb20 gene portion and a Zbtb20 promoter portion, wherein the Zbtb20 promoter portion comprises DNA sequences within, encompassing, and/or close to a Zbtb20 promoter.

37. The method of any one of the foregoing claims, wherein the protein is capable of binding to a Zbtb20 gene portion and is further capable of cleaving at least one DNA strand of the Zbtb20 gene portion.

38. The method of any one of the foregoing claims, wherein the protein is encoded by a nucleic acid.

39. The method of any one of the foregoing claims, wherein the nucleic acid encoding the protein is a construct comprising at least one promoter operatively linked to a nucleotide sequence encoding the protein, wherein the promoter is selected from a constitutive promoter and an inducible promoter.

40. The method of any one of the foregoing claims, wherein the construct is selected from a plasmid, a retrovirus construct, a lentivirus construct, an adenovirus construct, and an adeno-associated virus (AAV) construct.

41. The method of any one of the foregoing claims, wherein the nucleic acid encoding the protein is an in vitro transcribed mRNA.

42. The method of any one of the foregoing claims, wherein the protein and/or the nucleic acid encoding the protein is delivered to the modified cells prior to the administration of the modified cells to the subject.

43. The method of any one of the foregoing claims, wherein the protein is selected from a Cas9 and a Cpf1 (Cas12a).

44. The method of any one of the foregoing claims, wherein the modified cells further comprise at least one exogenous T cell receptor.

45. The method of any one of the foregoing claims, wherein the modified cells further comprise at least one CAR.

46. The method of any one of the foregoing claims, wherein the at least one cancer comprises one or more solid and/or hematological cancers in the subject.

47. The method of any one of the foregoing claims, wherein an amount of solid and/or hematological cancer cells in the subject is reduced and/or eliminated.

48. The method of any of the foregoing claims, wherein the at least one cancer is selected from one or more of adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, Kaposi's sarcoma, bladder cancer, bone cancer, brain cancer, breast cancer, carcinoid tumors, cervical cancer, chemotherapy-resistant cancer, colon cancer, endometrial cancer, esophageal cancer, gastric cancer, head cancer, neck cancer, hepatobiliary cancer, kidney cancer, leukemia, liver cancer, lung cancer, lymphoma, Hodgkin's disease, non-Hodgkin's lymphoma, metastatic cancer, nervous system tumors, oral cancer, ovarian cancer, pancreatic cancer, prostate cancer, rectal cancer, skin cancer, stomach cancer, testicular cancer, thyroid cancer, urethral cancer, cancer of bone marrow, multiple myeloma, tumors that metastasize to the bone, tumors infiltrating the nerve and hollow viscus, and tumors near neural structures.

49. The method of any one of the foregoing claims, wherein the modified cells are administered to the subject systemically or locally.

50. The method of any one of the foregoing claims, wherein the modified cells are administered by an injection method selected from intravenous, subcutaneous, intracavitary, intraventricular, intracranial, and intrathecal injection.

51. The method of any one of the foregoing claims, further comprising administering one or more additional cancer therapies to the subject.

52. The method of any one of the foregoing claims, wherein the additional cancer therapies comprise immunotherapy, chemotherapy, targeted therapy, stem cell transplant, radiation, surgery, and hormone therapy.

53. The method of any one of the foregoing claims, wherein the immunotherapy additionally comprises immune checkpoint inhibitors (e.g., negative checkpoint blockade, optionally a PD-1, PD-L1, or CTLA-4 antagonist antibody), monoclonal antibodies, cancer vaccines, immune system modulators, and adoptive cell therapies;
wherein the adoptive cell therapy is optionally selected from CAR T-cell therapy, CAR NK-cell therapy exogenous TCR therapy, and TIL therapy or a combination of any of the foregoing.

54. An isolated cell, wherein the cell is modified ex vivo to suppress endogenous Zbtb20 expression and/or activity within the cell.

55. The modified isolated cell of any one of the foregoing claims, wherein the modified isolated cell is an immune cell.

56. The modified isolated cell of any one of the foregoing claims, wherein the immune cell is selected from a T cell and a T cell progenitor.

57. The modified isolated cell of any one of the foregoing claims, wherein the immune cell is a NK cell.

58. The modified isolated cell of any one of the foregoing claims, wherein the immune cell is a CD8⁺ T cell or a CD8⁺ T cell progenitor.

59. The modified isolated cell of any one of the foregoing claims, wherein the immune cell is a CD4⁺ T cell or a CD4⁺ T cell progenitor.

60. The modified isolated cell of any one of the foregoing claims, wherein the immune cell is selected from a lymphocyte, a T cell, a NK cell, a B cell, a neutrophil (granulocyte), a monocyte, and a dendritic cell.

61. The modified isolated cell of any one of the foregoing claims, wherein the modified isolated cell is a mammalian cell selected from a rodent cell, a non-human primate cell, and a human cell.

62. The modified isolated cell of any one of the foregoing claims, comprising a dominant negative Zbtb20,
wherein the dominant negative Zbtb20 comprises one or more Zbtb20 C-terminal zinc-finger domains and lacks at least a portion of a Zbtb20 N-terminal region comprising a Zbtb20 BTB domain; and
wherein the dominant negative Zbtb20 suppresses endogenous Zbtb20 activity within the modified isolated cell.

63. The modified isolated cell of any one of the foregoing claims, wherein the dominant negative Zbtb20 is encoded by a nucleic acid comprising a nucleotide sequence which is at least 75% identical, at least 80% identical, at least 85% identical, at least 90% identical, at least 95% identical, at least 98% identical, or at least 99% identical to SEQ ID NO: 39 or SEQ ID NO: 41 or to another mammalian Zbtb20 nucleic acid coding sequence.

64. The modified isolated cell of any one of the foregoing claims, wherein the nucleic acid encoding the dominant negative Zbtb20 is a construct comprising at least one promoter,
wherein the at least one promoter is operatively linked to the nucleotide sequence; and
wherein the promoter is selected from a constitutive promoter and an inducible promoter.

65. The modified isolated cell of any one of the foregoing claims, wherein the construct is selected from a plasmid, a retrovirus construct, a lentivirus construct, an adenovirus construct, and an adeno-associated virus (AAV) construct.

66. The modified isolated cell of any one of the foregoing claims, wherein the nucleic acid encoding the dominant negative Zbtb20 is an in vitro transcribed mRNA.

67. The modified isolated cell of any one of the foregoing claims, wherein the dominant negative Zbtb20 and/or the nucleic acid encoding the dominant negative Zbtb20 is delivered to the isolated modified cell.

68. The modified isolated cell of any one of the foregoing claims, wherein the dominant negative Zbtb20 comprises an amino acid sequence which is at least 75% identical, at least 80% identical, at least 85% identical, at least 90% identical, at least 95% identical, at least 98% identical, or at least 99% identical to SEQ ID NO: 40 or SEQ ID NO: 42.

69. The modified isolated cell of any one of the foregoing claims, wherein the modified cell comprises at least one non-coding RNA capable of suppressing endogenous Zbtb20 expression within the modified isolated cell.

70. The modified isolated cell of any one of the foregoing claims, wherein the at least one non-coding RNA comprises at least one shRNA capable of suppressing endogenous Zbtb20 expression in the modified cells.

71. The modified isolated cell of any one of the foregoing claims, wherein the at least one shRNA is encoded by a nucleic acid comprising a nucleotide sequence selected from SEQ ID NO: 5, SEQ ID NO: 7, SEQ ID NO: 9, SEQ ID NO: 11, SEQ ID NO: 13, and SEQ ID NO: 15.

72. The modified isolated cell of any one of the foregoing claims, wherein the nucleic acid encoding at least one shRNA is a construct comprising at least one promoter,
wherein the at least one promoter is operatively linked to the nucleotide sequence; and
wherein the promoter is selected from a constitutive promoter and an inducible promoter.

73. The modified isolated cell of any one of the foregoing claims, wherein the construct is selected from a plasmid, a retrovirus construct, a lentivirus construct, an adenovirus construct, and an adeno-associated virus (AAV) construct.

74. The modified isolated cell of any one of the foregoing claims, wherein the at least one shRNA and/or the nucleic acid encoding at least one shRNA is delivered to the modified isolated cell.

75. The modified isolated cell of any one of the foregoing claims, wherein the at least one shRNA is selected from SEQ ID NO; 6, SEQ ID NO: 8, SEQ ID NO: 10, SEQ ID NO; 12, SEQ ID NO: 14, and SEQ ID NO: 16.

76. The modified isolated cell of any one of the foregoing claims, wherein the at least one non-coding RNA comprises at least one sgRNA capable of suppressing endogenous Zbtb20 expression in the modified cells.

77. The modified isolated cell of any one of the foregoing claims, wherein the at least one sgRNA is encoded by a nucleic acid comprising a nucleotide sequence selected from SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, and SEQ ID NO: 37.

78. The modified isolated cell of any one of the foregoing claims, wherein the nucleic acid encoding at least one sgRNA is a construct comprising at least one promoter,
wherein the at least one promoter is operatively linked to the nucleotide sequence; and
wherein the promoter is selected from a constitutive promoter and an inducible promoter.

79. The modified isolated cell of any one of the foregoing claims, wherein the construct is selected from a plasmid, a retrovirus construct, a lentivirus construct, an adenovirus construct, and an adeno-associated virus (AAV) construct.

80. The modified isolated cell of any one of the foregoing claims, wherein the at least one sgRNA and/or the nucleic acid encoding at least one sgRNA is delivered to the modified isolated cell.

81. The modified isolated cell of any one of the foregoing claims, wherein the at least one sgRNA is selected from SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, and SEQ ID NO: 38.

82. The modified isolated cell of any one of the foregoing claims, wherein the modified isolated cell further comprises a protein capable of binding to the sgRNA and to at least one of a Zbtb20 gene portion and a Zbtb20 promoter portion, wherein the Zbtb20 promoter portion comprises DNA sequences within, encompassing, and/or close to a Zbtb20 promoter.

83. The modified isolated cell of any one of the foregoing claims, wherein the protein is encoded by a nucleic acid.

84. The modified isolated cell of any one of the foregoing claims, wherein the nucleic acid is a construct comprising at least one promoter operatively linked to a nucleotide sequence encoding the protein, wherein the promoter is selected from a constitutive promoter and an inducible promoter.

85. The modified isolated cell of any one of the foregoing claims, wherein the construct is selected from a plasmid, a retrovirus construct, a lentivirus construct, an adenovirus construct, and an adeno-associated virus (AAV) construct.

86. The modified isolated cell of any one of the foregoing claims, wherein the nucleic acid encoding the protein is an in vitro transcribed mRNA.

87. The modified isolated cell of any one of the foregoing claims, wherein the protein and/or the nucleic acid encoding the protein is delivered to the modified isolated cell.

88. The modified isolated cell of any one of the foregoing claims, wherein the protein is selected from a Cas9 and a Cpf1 (Cas12a).

89. The modified isolated cell of any one of the foregoing claims, wherein the protein is capable of binding to a Zbtb20 gene portion and is further capable of cleaving at least one DNA strand of the Zbtb20 gene portion.

90. The modified isolated cell of any one of the foregoing claims, further comprising at least one exogenous T cell receptor.

91. The modified isolated cell of any one of the foregoing claims, further comprising at least one CAR.

92. A composition comprising one or more modified isolated cells of any one of the foregoing claims and a pharmaceutically acceptable carrier.

93. The composition of any one of the foregoing claims, further comprising at least one stabilizer.

94. The composition of any one of the foregoing claims, further comprising an additive that promotes an ability of the modified cell to cross the BBB, wherein the additive is optionally attached to or complexed with the modified cells.

95. A dominant negative Zbtb20, comprising one or more Zbtb20 C-terminal zinc-finger domains and lacking at least a portion of a Zbtb20 N-terminal region comprising a Zbtb20 BTB domain;
wherein the dominant negative Zbtb20 suppresses endogenous Zbtb20 activity; and
wherein the dominant negative Zbtb20 is derived from mouse Zbtb20 or human Zbtb20.

96. The dominant negative Zbtb20 of claim 95, comprising an amino acid sequence which is at least 75% identical, at least 80% identical, at least 85% identical, at least 90% identical, at least 95% identical, at least 98% identical, or at least 99% identical to SEQ ID NO: 40 or SEQ ID NO: 42 or to another mammalian Zbtb20 amino acid sequence.

97. A nucleic acid comprising a nucleotide sequence encoding the dominant negative Zbtb20 of claim 95 or 96.

98. The nucleic acid of claim 97, wherein the nucleotide sequence is at least 75% identical, at least 80% identical, at least 85% identical, at least 90% identical, at least 95% identical, at least 98% identical, or at least 99% identical to SEQ ID NO: 39 or SEQ ID NO: 41.

99. The nucleic acid of any one of claims 97-98, wherein the nucleic acid is a construct comprising at least one promoter,
wherein the at least one promoter is operatively linked to the nucleotide sequence; and
wherein the promoter is selected from a constitutive promoter and an inducible promoter.

100. The nucleic acid of claim 99, wherein the construct is selected from a plasmid, a retrovirus construct, a lentivirus construct, an adenovirus construct, and an adeno-associated virus (AAV) construct.

101. The nucleic acid of any one of claims 97-100, wherein the nucleic acid is an in vitro transcribed mRNA.

102. An shRNA capable of suppressing Zbtb20 expression.

103. The shRNA of claim 102, wherein the shRNA is selected from SEQ ID NO: 6, SEQ ID NO: 8, SEQ ID NO: 10, SEQ ID NO: 12, SEQ ID NO: 14, and SEQ ID NO: 16.

104. A nucleic acid comprising a nucleotide sequence encoding the shRNA of any one of claims 102-103.

105. The nucleic acid of claim 104, wherein the nucleotide sequence is selected from SEQ ID NO: 5, SEQ ID NO: 7, SEQ ID NO: 9, SEQ ID NO: 11, SEQ ID NO: 13, and SEQ ID NO: 15.

106. The nucleic acid of any one of claims 97-105, wherein the nucleic acid is a construct comprising at least one promoter,
wherein the at least one promoter is operatively linked to the nucleotide sequence; and
wherein the promoter is selected from a constitutive promoter and an inducible promoter.

107. The nucleic acid of claim 106, wherein the construct is selected from a plasmid, a retrovirus construct, a lentivirus construct, an adenovirus construct, and an adeno-associated virus (AAV) construct.

108. An sgRNA capable of binding to at least a portion of a Zbtb20 gene and suppressing Zbtb20 expression.

109. The sgRNA of claim 108, wherein the sgRNA is selected from SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32.

110. A nucleic acid comprising a nucleotide sequence encoding the sgRNA of any one of claims 108-109.

111. The nucleic acid of claim 110, wherein the nucleotide sequence is selected from SEQ ID NO: 17, SEQ ID NO: 19, SEQ ID NO: 21, SEQ ID NO: 23, SEQ ID NO: 25, SEQ ID NO: 27, SEQ ID NO: 29, and SEQ ID NO: 31.

112. The nucleic acid of any one of claims 110-111, wherein the nucleic acid is a construct comprising at least one promoter,
wherein the at least one promoter is operatively linked to the nucleotide sequence; and
wherein the promoter is selected from a constitutive promoter and an inducible promoter.

113. The nucleic acid of claim 112, wherein the construct is selected from a plasmid, a retrovirus construct, a lentivirus construct, an adenovirus construct, and an adeno-associated virus (AAV) construct.
